(19) United States
(12) Patent Application Publication
(10) Pub. No.: US 2023/0138673 A1
(43) Pub. Date: May 4, 2023

(54) Ranking Feedback For Improving Diabetes Management

(71) Applicant: DexCom, Inc., San Diego, CA (US)

(72) Inventors: Margaret A. Crawford, Encinitas, CA (US); Mark Derdzinski, La Jolla, CA (US); Giada Acciaroli, Edinburgh (GB); Robert J. Dowd, San Diego, CA (US); Lauren H. Jepson, San Diego, CA (US); Sarah Kate Pickus, San Diego, CA (US); Apurv U. Kamath, San Diego, CA (US)

(73) Assignee: Dexcom, Inc., San Diego, CA (US)

(21) Appl. No.: 17/974,299

(22) Filed: Oct. 26, 2022

Related U.S. Application Data
(60) Provisional application No. 63/263,188, filed on Oct. 28, 2021.

(51) Int. Cl. A61B 5/145; G16H 20/10; G16H 50/20; A61B 5/00

(52) U.S. Cl. CPC A61B 5/14532; G16H 20/10; G16H 50/20; A61B 5/746

(57) ABSTRACT

Feedback regarding diabetes management by a user is generated, such as feedback identifying improvements in glucose measurements for a given time period over previous days, feedback identifying sustained positive patterns, feedback identifying deviations in glucose measurements between time periods, feedback identifying potential behavior modification that a user could take to engage in beneficial diabetes management behavior, feedback identifying what a user's glucose would have been had the particular events or conditions not occurred or not been present, and so forth. A feedback presentation system analyzes the identified feedback and selects feedback based on various rankings, rules and conditions for display to the user. The selected feedback is provided to the user at various times, such as regular reports (e.g., daily or weekly reports), in real time (e.g., notifying the user what his glucose level would have been had he not just taken a walk), and so forth.

### RELATED APPLICATION

This application claims the benefit of U.S. Provisional Patent Application No. 63/263,188, filed Oct. 28, 2021, and titled “Ranking Feedback For Improving Diabetes Management,” the entire disclosure of which is hereby incorporated by reference.

### BACKGROUND

Diabetes is a metabolic condition affecting hundreds of millions of people and is one of the leading causes of death worldwide. For people living with Type I diabetes, access to treatment is critical to their survival and it can reduce adverse outcomes among people with Type II diabetes. With proper treatment, serious damage to the heart, blood vessels, eyes, kidneys, and nerves due to diabetes can be avoided. Regardless of the type of diabetes (e.g., Type I or Type II), managing diabetes successfully involves monitoring and oftentimes adjusting food and activity to control a person's blood glucose, such as to reduce severe fluctuations in and/or generally lower the person's glucose.

However, many conventional glucose monitoring applications employ user interfaces that display raw glucose information in a manner that is difficult for users to interpret, particularly users who have just recently started monitoring their glucose. Consequently, users may be unable to draw insights from the data and thus are unable to alter their behavior in a meaningful way in order to improve their glucose. Over time, these users often become overwhelmed and frustrated by the manner in which information is presented by these conventional glucose monitoring applications and thus discontinue use of these applications before improvements in their glucose and overall health can be realized. Moreover, as users increasingly utilize mobile devices (e.g., smart watches and smart phones) to access glucose monitoring information, the failure by conventional systems to provide meaningful glucose information in a manner that users can understand is further exacerbated by the constraints imposed by the small screens of these mobile devices.

### SUMMARY

To overcome these problems, techniques for ranking feedback for improving diabetes management are discussed. In one or more implementations, in a diabetes management monitoring system, diabetes management measurements are obtained from a sensor of the diabetes management monitoring system. Multiple diabetes management feedbacks are identified, based on the diabetes management measurements, that correspond to the diabetes management measurements. One or more of the multiple diabetes management feedbacks having a highest ranking is determined, and the determined diabetes management feedback is caused to be displayed.

This Summary introduces a selection of concepts in a simplified form that are further described below in the Detailed Description. As such, this Summary is not intended to identify essential features of the claimed subject matter, nor is it intended to be used as an aid in determining the scope of the claimed subject matter.

### BRIEF DESCRIPTION OF THE DRAWINGS

The detailed description is described with reference to the accompanying figures.

FIG. 1 is an illustration of an environment in an example of an implementation that is operable to implement ranking feedback for improving diabetes management as described herein.

FIG. 2 depicts an example of an implementation of a wearable glucose monitoring device.

FIG. 3 is an illustration of an example architecture of a system implementing the techniques discussed herein.

FIG. 4 is an illustration of an example architecture of a diabetes management feedback generation system.

FIG. 5 illustrates an example of providing feedback indicating improvement in at least one feature for a time period.

FIG. 6 illustrates an example of providing feedback indicating a best time period during a day.

FIG. 7 illustrates an example of providing feedback indicating a sustained positive pattern.

FIG. 8 depicts a procedure in an example of implementing diabetes management feedback for improving diabetes management.

FIG. 9 depicts a procedure in another example of implementing diabetes management feedback for improving diabetes management.

FIG. 10 is an illustration of an example architecture of a glucose level deviation detection system.

FIG. 11 is an illustration of an example implementation of the content-based deviation detection module.

FIG. 12 illustrates an example of generating a deviation indication.

FIG. 13 depicts a procedure in an example of implementing glucose level deviation detection.

FIG. 14 depicts a procedure in another example of implementing glucose level deviation detection.

FIG. 15 is an illustration of an example architecture of a behavior modification identification system.

FIG. 16 illustrates an example of providing behavior modification recommendations for improving diabetes management.

FIG. 17 illustrates an example of sizes of normalized sizes for different detected patterns.

FIG. 18 describes an example procedure for implementing behavior modification feedback for improving diabetes management.

FIG. 19 is an illustration of an example architecture of a glucose prediction system.

FIG. 20 illustrates an example of generating predicted glucose measurements.

FIG. 21 illustrates an example of providing predicted glucose measurements.

FIG. 22 depicts a procedure in an example of implementing glycemic impact prediction for improving diabetes management.

FIG. 23 depicts a procedure in an example of implementing glycemic impact prediction for improving diabetes management.

FIG. 24 illustrates an example of feedback.

FIG. 25 describes an example of a procedure for implementing ranking feedback for improving diabetes management.

FIG. 26 illustrates an example of a system that includes an example of a computing device that is representative of one or more computing systems and/or devices that may implement the various techniques described herein.

### DETAILED DESCRIPTION

Overview

Techniques for ranking feedback for improving diabetes management are discussed herein. Broadly, blood glucose level measurements of a user are obtained over time. Glucose level measurements are typically obtained by a wearable glucose monitoring device being worn by the user. These glucose level measurements can be produced substantially continuously, such that the device may be configured to produce the glucose level measurements at regular or irregular intervals of time (e.g., approximately every hour, approximately every 30 minutes, approximately every 5 minutes, and so forth), responsive to establishing a communicative coupling with a different device (e.g., when a computing device establishes a wireless connection with a wearable glucose level monitoring device to retrieve one or more of the measurements), and so forth. These glucose level measurements are analyzed based on various rules to determine time periods of good (or optionally bad) diabetes management by the user and feedback indicating such is provided to the user.

Various different feedback regarding diabetes management by a user are generated, such as feedback identifying improvements in glucose measurements for a given time period over one or more previous days, feedback identifying a time period of the day during which glucose measurements were the best (e.g., within an optimal range or closest to an optimal value), feedback identifying sustained positive patterns (e.g., good diabetes management for a same time period in each of multiple days), feedback identifying deviations in glucose measurements between time periods, feedback identifying potential behavior modification (e.g., actions) that a user could take to engage in beneficial diabetes management behavior, feedback identifying what a user's glucose would have been had the particular events or conditions not occurred or not been present (e.g., the user had not taken a walk), and so forth.

Given the large amount of feedback that can be generated, a feedback presentation system analyzes the identified feedback and selects feedback based on various rankings, rules, and conditions for display to the user. The feedback presentation system can provide the selected feedback to the user at various times, such as at regular intervals (e.g., daily or weekly reports), in real time (e.g., notifying the user what his glucose level would have been had he not just taken a walk), and so forth.

The techniques discussed herein improve the user interface presented to the user by reducing the amount of feedback (the number of items of feedback at once or over time) provided to the user. Large amounts of feedback can be available for presentation to the user, but systems that present too much feedback can quickly overwhelm users. In such situations, feedback that may be informative and improve diabetes management if acted upon by the user becomes lost and not acted upon by the user due to the overwhelming amount of feedback. Furthermore, the large amount of feedback can desensitize users, again resulting in feedback that may improve diabetes management if acted upon actually being ignored and not being acted upon by the user. Thus, in contrast to systems that provide too much feedback, the techniques discussed herein reduce the amount of feedback provided to users, improving the diabetes management of the users by increasing the likelihood of the users acting on the feedback and increasing their short term or long term health.

The techniques discussed herein further improve the user interface presented to the user by presenting feedback in a manner that is easily interpretable by the user—rather than (or in addition to) raw glucose data, feedback highlighting actions taken by the user that benefited their glucose, suggesting other actions that could be taken to improve diabetes management, and so forth are provided to the user.

The techniques discussed herein further reduce repetitiveness in the feedback provided to the user (e.g., the same feedback is not displayed every day) while at the same time providing personalized feedback to the user (e.g., the highest ranked feedback). Repeatedly providing the same or similar feedback can desensitize users to the feedback, resulting in feedback that may be informative and improve diabetes management if acted upon actually being ignored and not being acted upon by the user. Thus, reducing the repetitiveness and increasing the personalization of the feedback provided to the user improves user interaction with the computing device and improves the diabetes management of the user by increasing the likelihood of the user acting on the feedback and increasing their short term or long term health.

In the following discussion, an example environment is first described that may employ the techniques described herein. Examples of implementation details and procedures are then described which may be performed in the example environment as well as other environments. Performance of the example procedures is not limited to the example environment and the example environment is not limited to performance of the example procedures.

Example of an Environment

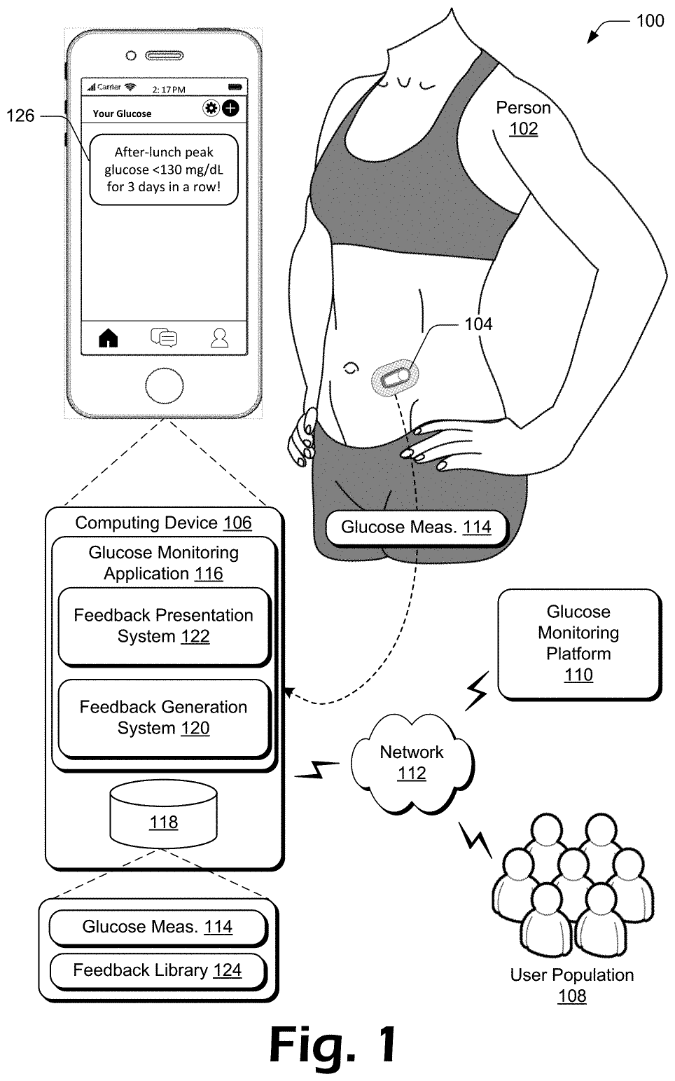

FIG. 1 is an illustration of an environment 100 in an example of an implementation that is operable to implement ranking feedback for improving diabetes management as described herein. The illustrated environment 100 includes a person 102, who is depicted wearing a wearable glucose monitoring device 104. The illustrated environment 100 also includes a computing device 106, other users in a user population 108 that wear glucose monitoring devices 104, and a glucose monitoring platform 110. The wearable glucose monitoring device 104, computing device 106, user population 108, and glucose monitoring platform 110 are communicatively coupled, including via a network 112.

Alternately or additionally, the wearable glucose monitoring device 104 and the computing device 106 may be communicatively coupled in other ways, such as using one or more wireless communication protocols or techniques. By way of example, the wearable glucose monitoring device 104 and the computing device 106 may communicate with one another using one or more of Bluetooth (e.g., Bluetooth Low Energy links), near-field communication (NFC), 5G, and so forth.

In accordance with the described techniques, the wearable glucose monitoring device 104 is configured to provide measurements of person 102′s glucose. Although a wearable glucose monitoring device is discussed herein, it is to be appreciated that user interfaces for glucose monitoring may be generated and presented in connection with other devices capable of providing glucose measurements, e.g., non-wearable glucose devices such as blood glucose meters requiring finger sticks, patches, and so forth. In implementations that involve the wearable glucose monitoring device 104, though, it may be configured with a glucose sensor that continuously detects analytes indicative of the person 102′s glucose and enables generation of glucose measurements. In the illustrated environment 100 and throughout the detailed description these measurements are represented as glucose measurements 114.

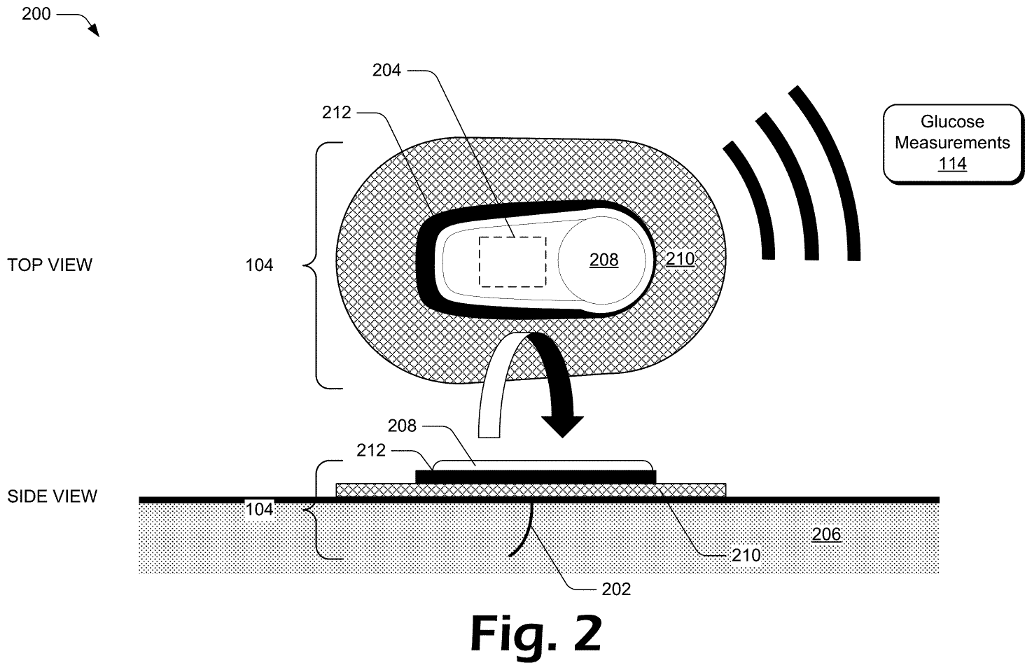

In one or more implementations, the wearable glucose monitoring device 104 is a continuous glucose monitoring (“CGM”) system. As used herein, the term “continuous” used in connection with glucose monitoring may refer to an ability of a device to produce measurements substantially continuously, such that the device may be configured to produce the glucose measurements 114 at regular or irregular intervals of time (e.g., every hour, every 30 minutes, every 5 minutes, and so forth), responsive to establishing a communicative coupling with a different device (e.g., when a computing device establishes a wireless connection with the wearable glucose monitoring device 104 to retrieve one or more of the measurements), and so forth. This functionality along with further aspects of the wearable glucose monitoring device 104's configuration is discussed in more detail in relation to FIG. 2.

Additionally, the wearable glucose monitoring device 104 transmits the glucose measurements 114 to the computing device 106, such as via a wireless connection. The wearable glucose monitoring device 104 may communicate these measurements in real-time, e.g., as they are produced using a glucose sensor. Alternately or in addition, the wearable glucose monitoring device 104 may communicate the glucose measurements 114 to the computing device 106 at set time intervals. For example, the wearable glucose monitoring device 104 may be configured to communicate the glucose measurements 114 to the computing device 106 every five minutes (as they are being produced).

Certainly, an interval at which the glucose measurements 114 are communicated may be different from the examples above without departing from the spirit or scope of the described techniques. The measurements may be communicated by the wearable glucose monitoring device 104 to the computing device 106 according to other bases in accordance with the described techniques, such as based on a request from the computing device 106. Regardless, the computing device 106 may maintain the glucose measurements 114 of the person 102 at least temporarily, e.g., in computer-readable storage media of the computing device 106.

Although illustrated as a mobile device (e.g., a mobile phone), the computing device 106 may be configured in a variety of ways without departing from the spirit or scope of the described techniques. By way of example and not limitation, the computing device 106 may be configured as a different type of device, such as a mobile device (e.g., a wearable device, tablet device, or laptop computer), a stationary device (e.g., a desktop computer), an automotive computer, and so forth. In one or more implementations, the computing device 106 may be configured as a dedicated device associated with the glucose monitoring platform 110, e.g., with functionality to obtain the glucose measurements 114 from the wearable glucose monitoring device 104, perform various computations in relation to the glucose measurements 114, display information related to the glucose measurements 114 and the glucose monitoring platform 110, communicate the glucose measurements 114 to the glucose monitoring platform 110, and so forth.

Additionally, the computing device 106 may be representative of more than one device in accordance with the described techniques. In one or more scenarios, for instance, the computing device 106 may correspond to both a wearable device (e.g., a smart watch) and a mobile phone. In such scenarios, both of these devices may be capable of performing at least some of the same operations, such as to receive the glucose measurements 114 from the wearable glucose monitoring device 104, communicate them via the network 112 to the glucose monitoring platform 110, display information related to the glucose measurements 114, and so forth. Alternately or in addition, different devices may have different capabilities that other devices do not have or that are limited through computing instructions to specified devices.

In the scenario where the computing device 106 corresponds to a separate smart watch and a mobile phone, for instance, the smart watch may be configured with various sensors and functionality to measure a variety of physiological markers (e.g., heartrate, heartrate variability, breathing, rate of blood flow, and so on) and activities (e.g., steps or other exercise) of the person 102. In this scenario, the mobile phone may not be configured with these sensors and functionality, or it may include a limited amount of that functionality—although in other scenarios a mobile phone may be able to provide the same functionality. Continuing with this particular scenario, the mobile phone may have capabilities that the smart watch does not have, such as a camera to capture images associated with glucose monitoring and an amount of computing resources (e.g., battery and processing speed) that enables the mobile phone to more efficiently carry out computations in relation to the glucose measurements 114. Even in scenarios where a smart watch is capable of carrying out such computations, computing instructions may limit performance of those computations to the mobile phone so as not to burden both devices and to utilize available resources efficiently. To this extent, the computing device 106 may be configured in different ways and represent different numbers of devices than discussed herein without departing from the spirit and scope of the described techniques.

In accordance with the discussed techniques, the computing device 106 is configured to implement ranking feedback for improving diabetes management. In the environment 100, the computing device 106 includes glucose monitoring application 116 and storage device 118. Here, the glucose monitoring application 116 includes a diabetes management feedback generation system 120 and a diabetes management feedback presentation system 122. Although illustrated as being included in computing device 106, additionally or alternatively at least some functionality of one or both of the diabetes management feedback generation system 120 and the diabetes management feedback presentation system 122 is located elsewhere, such as in glucose monitoring platform 110. Further, the glucose measurements 114 and feedback library 124 are shown stored in the storage device 118. The storage device 118 may represent one or more databases and also other types of storage capable of storing the glucose measurements 114 and the feedback library 124. The feedback library 124 stores multiple feedback items (e.g., messages or message templates) that can be provided to the user 102, for example to highlight the positive impacts of recent diabetes management choices for reflection and motivation by the user 102, to identify deviations, to identify goals, to identify what glucose measurements had particular activities not been taken, and so forth.

In one or more implementations, the glucose measurements 114 and/or the feedback library 124 may be stored at least partially remote from the computing device 106, e.g., in storage of the glucose monitoring platform 110, and retrieved or otherwise accessed in connection with configuring and outputting (e.g., displaying) user interfaces for diabetes management feedback presentation. For instance, the glucose measurements 114 and/or the feedback library 124 may be generally stored in storage of the glucose monitoring platform 110 along with the glucose measurements of the user population 108 and/or the feedback library 124, and some of that data may be retrieved or otherwise accessed on an as-needed basis to display user interfaces for diabetes management feedback presentation.

Broadly speaking, the glucose monitoring application 116 is configured to support interactions with a user that enable feedback about the user's glucose to be presented. This may include, for example, obtaining the glucose measurements 114 for processing (e.g., to determine the appropriate feedback), receiving information about a user (e.g., through an onboarding process and/or user feedback), causing information to be communicated to a health care provider, causing information to be communicated to the glucose monitoring platform 110, and so forth.

In one or more implementations, the glucose monitoring application 116 also leverages resources of the glucose monitoring platform 110 in connection with ranking feedback for improving diabetes management. As noted above, for instance, the glucose monitoring platform 110 may be configured to store data, such as the glucose measurements 114 associated with a user (e.g., the person 102) and/or users of the user population 108, and the feedback library 124. The glucose monitoring platform 110 may also provide updates and/or additions to the glucose monitoring application 116. Further still, the glucose monitoring platform 110 may train, maintain, and/or deploy algorithms (e.g., machine learning algorithms) to generate or select feedback or to identify time periods for which feedback is provided, such as by using the wealth of data collected from the person 102 and the users of the user population 108. One or more such algorithms may require an amount of computing resources that exceeds the resources of typical, personal computing devices, e.g., mobile phones, laptops, tablet devices, and wearables, to name just a few. Nonetheless, the glucose monitoring platform 110 may include or otherwise have access to the amount of resources needed to operate such algorithms, e.g., cloud storage, server devices, virtualized resources, and so forth. The glucose monitoring platform 110 may provide a variety of resources that the glucose monitoring application 116 leverages in connection with enabling diabetes management feedback to be presented via user interfaces.

In accordance with the described techniques, the diabetes management feedback generation system 120 is configured to use the glucose measurements 114 to identify one or more feedback items in the feedback library 124 and the diabetes management feedback presentation system 122 is configured to cause output of one or more user interfaces that present the identified diabetes management feedback. The glucose monitoring application 116 may cause display of the configured user interface 126 via a display device of the computing device 106 or other display device.

As discussed above and below, a variety of diabetes management feedback (e.g., messages) may be selected or generated based on the glucose measurements 114 of the user in accordance with the described techniques. In the context of measuring glucose, e.g., continuously, and obtaining data describing such measurements, consider the following discussion of FIG. 2.

FIG. 2 depicts an example 200 of an implementation of the wearable glucose monitoring device 104 of FIG. 1 in greater detail. In particular, the illustrated example 200 includes a top view and a corresponding side view of the wearable glucose monitoring device 104. It is to be appreciated that the wearable glucose monitoring device 104 may vary in implementation from the following discussion in various ways without departing from the spirit or scope of the described techniques. As noted above, for instance, user interfaces including diabetes management feedback presentation may be configured and displayed (or otherwise output) in connection with other types of devices for glucose monitoring, such as non-wearable devices (e.g., blood glucose meters requiring finger sticks), patches, and so forth.

In this example 200, the wearable glucose monitoring device 104 is illustrated to include a sensor 202 and a sensor module 204. Here, the sensor 202 is depicted in the side view having been inserted subcutaneously into skin 206, e.g., of the person 102. The sensor module 204 is depicted in the top view as a dashed rectangle. The wearable glucose monitoring device 104 also includes a transmitter 208 in the illustrated example 200. Use of the dashed rectangle for the sensor module 204 indicates that it may be housed or otherwise implemented within a housing of the transmitter 208. In this example 200, the wearable glucose monitoring device 104 further includes adhesive pad 210 and attachment mechanism 212.

In operation, the sensor 202, the adhesive pad 210, and the attachment mechanism 212 may be assembled to form an application assembly, where the application assembly is configured to be applied to the skin 206 so that the sensor 202 is subcutaneously inserted as depicted. In such scenarios, the transmitter 208 may be attached to the assembly after application to the skin 206 via the attachment mechanism 212. Alternatively, the transmitter 208 may be incorporated as part of the application assembly, such that the sensor 202, the adhesive pad 210, the attachment mechanism 212, and the transmitter 208 (with the sensor module 204) can all be applied at once to the skin 206. In one or more implementations, this application assembly is applied to the skin 206 using a separate sensor applicator (not shown). Unlike the finger sticks required by conventional blood glucose meters, the user initiated application of the wearable glucose monitoring device 104 is nearly painless and does not require the withdrawal of blood. Moreover, the automatic sensor applicator generally enables the person 102 to embed the sensor 202 subcutaneously into the skin 206 without the assistance of a clinician or healthcare provider.

The application assembly may also be removed by peeling the adhesive pad 210 from the skin 206. It is to be appreciated that the wearable glucose monitoring device 104 and its various components as illustrated are simply one example form factor, and the wearable glucose monitoring device 104 and its components may have different form factors without departing from the spirit or scope of the described techniques.

In operation, the sensor 202 is communicatively coupled to the sensor module 204 via at least one communication channel which can be a wireless connection or a wired connection. Communications from the sensor 202 to the sensor module 204 or from the sensor module 204 to the sensor 202 can be implemented actively or passively and these communications can be continuous (e.g., analog) or discrete (e.g., digital).

The sensor 202 may be a device, a molecule, and/or a chemical which changes or causes a change in response to an event which is at least partially independent of the sensor 202. The sensor module 204 is implemented to receive indications of changes to the sensor 202 or caused by the sensor 202. For example, the sensor 202 can include glucose oxidase which reacts with glucose and oxygen to form hydrogen peroxide that is electrochemically detectable by the sensor module 204 which may include an electrode. In this example, the sensor 202 may be configured as or include a glucose sensor configured to detect analytes in blood or interstitial fluid that are indicative of diabetes management using one or more measurement techniques. In one or more implementations, the sensor 202 may also be configured to detect analytes in the blood or the interstitial fluid that are indicative of other markers, such as lactate levels, which may improve accuracy in generating various diabetes management feedback. Additionally or alternately, the wearable glucose monitoring device 104 may include additional sensors to the sensor 202 to detect those analytes indicative of the other markers.

In another example, the sensor 202 (or an additional sensor of the wearable glucose monitoring device 104—not shown) can include a first and second electrical conductor and the sensor module 204 can electrically detect changes in electric potential across the first and second electrical conductor of the sensor 202. In this example, the sensor module 204 and the sensor 202 are configured as a thermocouple such that the changes in electric potential correspond to temperature changes. In some examples, the sensor module 204 and the sensor 202 are configured to detect a single analyte, e.g., glucose. In other examples, the sensor module 204 and the sensor 202 are configured to detect multiple analytes, e.g., sodium, potassium, carbon dioxide, and glucose. Alternately or additionally, the wearable glucose monitoring device 104 includes multiple sensors to detect not only one or more analytes (e.g., sodium, potassium, carbon dioxide, glucose, and insulin) but also one or more environmental conditions (e.g., temperature). Thus, the sensor module 204 and the sensor 202 (as well as any additional sensors) may detect the presence of one or more analytes, the absence of one or more analytes, and/or changes in one or more environmental conditions.

In one or more implementations, the sensor module 204 may include a processor and memory (not shown). The sensor module 204, by leveraging the processor, may generate the glucose measurements 114 based on the communications with the sensor 202 that are indicative of the above-discussed changes. Based on these communications from the sensor 202, the sensor module 204 is further configured to generate communicable packages of data that include at least one glucose measurement 114. In one or more implementations, the sensor module 204 may configure those packages to include additional data, including, by way of example and not limitation, a sensor identifier, a sensor status, temperatures that correspond to the glucose measurements 114, measurements of other analytes that correspond to the glucose measurements 114, and so forth. It is to be appreciated that such packets may include a variety of data in addition to at least one glucose measurement 114 without departing from the spirit or scope of the described techniques.

In implementations where the wearable glucose monitoring device 104 is configured for wireless transmission, the transmitter 208 may transmit the glucose measurements 114 wirelessly as a stream of data to a computing device. Alternately or additionally, the sensor module 204 may buffer the glucose measurements 114 (e.g., in memory of the sensor module 204 and/or other physical computer-readable storage media of the wearable glucose monitoring device 104) and cause the transmitter 208 to transmit the buffered glucose measurements 114 later at various intervals, e.g., time intervals (every second, every thirty seconds, every minute, every five minutes, every hour, and so on), storage intervals (when the buffered glucose measurements 114 reach a threshold amount of data or a number of measurements), and so forth.

Having considered an example of an environment and an example of a wearable glucose monitoring device, consider now a discussion of some examples of details of the techniques for ranking feedback for improving diabetes management.

### System Architecture

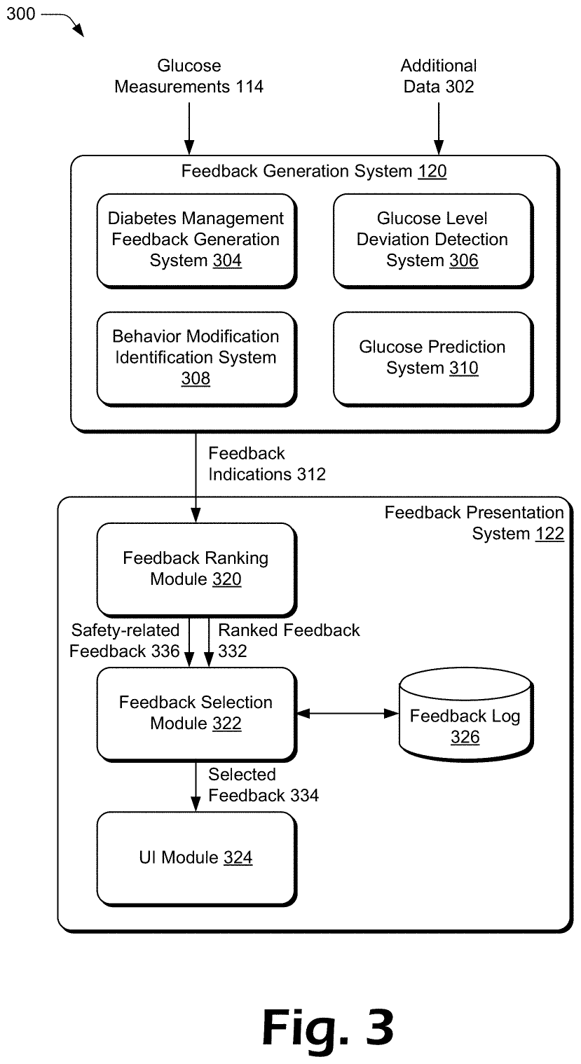

FIG. 3 is an illustration of an example architecture of a system 300 implementing the techniques discussed herein. The system 300 includes the feedback generation system 120 and the feedback presentation system 122. In the illustrated example, the feedback generation system 120 receives glucose measurements 114 and additional data 302. This additional data 302 can be any of a variety of different data as discussed in more detail below, such as activity data, meal data, medication data, and so forth. The feedback generation system 120 includes a diabetes management feedback generation system 304, a glucose level deviation detection system 306, a behavior modification identification system 308, and a glucose prediction system 310. Each of the systems 304—310 analyzes the glucose measurements 114, and optionally the additional data 302, to generate diabetes management feedback indications 312, also referred to as glucose management feedback indications or simply feedback indications. The feedback indications 312 are indications of diabetes management feedback or glucose management feedback for the user 102. The feedback indications 312 can take various forms, such as feedback (e.g., particular text) to provide to the user 102, the results of analyzing the glucose measurements 114 and optionally the additional data 302 to allow the feedback presentation system 122 to determine which feedback (e.g., particular text) to provide to the user 102, and so forth. The systems 304 — 310 can generate the feedback indications 312 in various different manners, as discussed in more detail below.

Although the feedback generation system 120 is illustrated as including the diabetes management feedback generation system 304, the glucose level deviation detection system 306, the behavior modification identification system 308, and the glucose prediction system 310, the feedback generation system 120 need not include all of the systems 304, 306, 308, and 310. For example, the feedback generation system 120 can include a subset of (e.g., two or three of) the diabetes management feedback generation system 304, the glucose level deviation detection system 306, the behavior modification identification system 308, and the glucose prediction system 310. Additionally or alternatively, the feedback generation system 120 can include additional feedback generation systems.

The diabetes management feedback generation system 304 is configured to use the glucose measurements 114 and optionally the additional data 302 to generate feedback indications 312. Generally, the diabetes management feedback generation system 304 analyzes the glucose measurements 114 for the user 102 and looks for patterns in the glucose measurements 114 that indicate good diabetes management by the user. Good diabetes management can be quantified in various manners, such as glucose measurements 114 staying within a particular range, glucose measurements 114 not varying by greater than a particular amount, and so forth. In one or more implementations, the diabetes management feedback generation system 304 identifies good diabetes management by identifying improvements in glucose measurements 114 for a given time period over one or more previous days, identifying a time period of the day during which glucose measurements 114 were the best (e.g., within an optimal range or closest to an optimal value), identifying sustained positive patterns (e.g., good diabetes management for a same time period in each of multiple days), or combinations thereof. The diabetes management feedback generation system 304 includes in feedback indications 312 feedback indicating good diabetes management or data from which the feedback presentation system 122 can generate feedback indicating good diabetes management. This feedback is provided for reflection and motivation by the user, oftentimes providing inspirational insights to the user to continue making good diabetes management to improve their health, prolong their life, and so forth. This feedback also educates the user on making good diabetes management choices, for example by allowing the user to mimic his or her changes from one time period that showed improvement (e.g., increasing the amount of vegetables he or she ate) to other time periods.

The glucose level deviation detection system 306 is configured to use the glucose measurements 114 and optionally the additional data 302 to identify deviations in the glucose level of the user and to generate feedback indications 312. Generally, the glucose level deviation detection system 306 analyzes the glucose measurements 114 for the user 102 and looks for deviations from a norm for the user. These deviations from the norm can be based on various factors, such as the user's current or recent glucose level relative to the user's glucose levels earlier in the day, the user's current or recent glucose level relative to the user's glucose levels in corresponding times of previous days, and so forth. Upon detection of one or more deviations, the glucose level deviation detection system 306 includes in feedback indications 312 feedback for the user (such as an identification of the deviation) or data from which the feedback presentation system 122 can generate feedback for the user.

The behavior modification identification system 308 is configured to use the glucose measurements 114 to generate feedback indications 312. Behavior modification feedback, also referred to as an actionable goal, refers to one or more actions that the user can take to alter (e.g., improve) his or her diabetes management. Generally, the behavior modification identification system 308 analyzes the glucose measurements 114 for the user 102 and looks for patterns in the glucose measurements 114 that indicate poor (or non-optimal) diabetes management by the user. Poor diabetes management can be quantified in various manners, such as glucose measurements 114 not staying within a particular range, glucose measurements 114 varying by greater than a particular amount, and so forth. In one or more implementations, the behavior modification identification system 308 identifies poor diabetes management by identifying patterns in glucose measurements 114 for a given time period of a time window across multiple time windows (e.g., for a given multi-hour time period, such as 6 am to noon, on each of multiple days). The behavior modification identification system 308 identifies behavior modification feedback corresponding to the identified poor diabetes management. The behavior modification identification system 308 includes in feedback indications 312 feedback corresponding to poor diabetes management identified by the behavior modification identification system 308 or data from which the feedback presentation system 122 can generate feedback corresponding to poor diabetes management identified by the behavior modification identification system 308.

The glucose prediction system 310 is configured to use the glucose measurements 114 and optionally the additional data 302 to generate feedback indications 312. Generally, the glucose prediction system 310 analyzes activity data of a user and determines when a period of physical activity occurs. The glucose prediction system 310 predicts what the glucose measurements of the user 102 would have been had the physical activity not occurred, and takes various actions based on the predicted glucose measurements (e.g., generates feedback for the user indicating what their glucose would have been had they not engaged in the physical activity). The glucose prediction system 310 includes in feedback indications 312 feedback for the user indicating what their glucose would have been had they not engaged in the physical activity or data from which the feedback presentation system 122 can generate feedback for the user indicating what their glucose would have been had they not engaged in the physical activity.

The feedback presentation system 122 receives the feedback indications 312 generated by the systems 304—310. Generally, the feedback presentation system 122 causes output of one or more user interfaces that present the diabetes management feedback indicated by the feedback indications 312. The feedback presentation system 122 includes a feedback ranking module 320, a feedback selection module 322, a UI module 324, and a feedback log 326. The feedback ranking module 320 ranks the various feedback indicated by the feedback indications 312 and provides the ranked feedback 332 to the feedback selection module 322. The feedback selection module 322 selects one or more of the ranked feedback 332 and provides the selected feedback 334 to the UI module 324. The feedback log 326 logs what feedback has been delivered to users historically and allows for adjusting ranked feedback to make delivered insights non-repetitive.

The UI module 324 receives the selected feedback 334 and causes the selected feedback 334 to be displayed or otherwise presented (e.g., at computing device 106). This display or other presentation can take various forms, such as a static text display, graphic or video display, audio presentation, combinations thereof, and so forth.

### Diabetes Management Feedback Generation System Architecture

Generally, the diabetes management feedback generation system 304 receives a data stream of glucose measurements. Various other data streams can also optionally be received, such as activity data (e.g., number of steps taken by the user). One or more features for a particular time period are generated and stored, each feature being a value that can be computed from data in a data stream and that indicates whether the user has been engaging in beneficial diabetes management behaviors or lifestyle choices. The features may include metrics that are a representation or summarization of the data in the data stream for a particular time period are generated and stored. These time periods are, for example, different multi-hour blocks of time during a day. E.g., a day may include a first time period from midnight to 6 am (corresponding to sleep), a second time period from 6 am to noon (corresponding to after breakfast), a third time period from noon to 6 pm (corresponding to after lunch), and a fourth time period from 6 pm to midnight (corresponding to after dinner). These time periods may be fixed or may be adaptively identified based on features identified in the different data streams (e.g., sleep onset may be detected by an activity monitor and may be used to determine the beginning of the “sleep” time period on that date).

Good diabetes management is identified in various manners, such as by identifying improvements in glucose measurements for a given time period over one or more previous days, identifying a time period of the day during which glucose measurements were the best (e.g., within an optimal range or closest to an optimal value), identifying sustained positive patterns (e.g., good diabetes management for a same time period in each of multiple days), and so forth. Once identified, feedback is generated that notifies the user of these improvements in glucose measurements, best time period of the day, sustained positive patterns, combinations thereof, and so forth. This feedback can be retrospective, e.g., informing the user how he or she did on diabetes management during the previous day or at the end of the current day.

The techniques discussed herein apply analogously to identify bad diabetes management. Poor diabetes management can be identified and feedback (e.g., warnings or negative affects) displayed or otherwise presented to the user to encourage better diabetes management by the user. This feedback can be used to identify problem areas that need to be addressed, actions or choices that should be avoided in the future, and so forth.

The techniques discussed herein generate feedback for the user that notifies the user of good diabetes management choices (or bad diabetes management choices) he or she has made. This feedback is provided for reflection and motivation by the user, oftentimes providing inspirational insights to the user to continue making good diabetes management to improve their health, prolong their life, and so forth. This feedback also educates the user on making good diabetes management choices, for example by allowing the user to mimic his or her changes from one time period that showed improvement (e.g., increasing the amount of vegetables he or she ate) to other time periods.

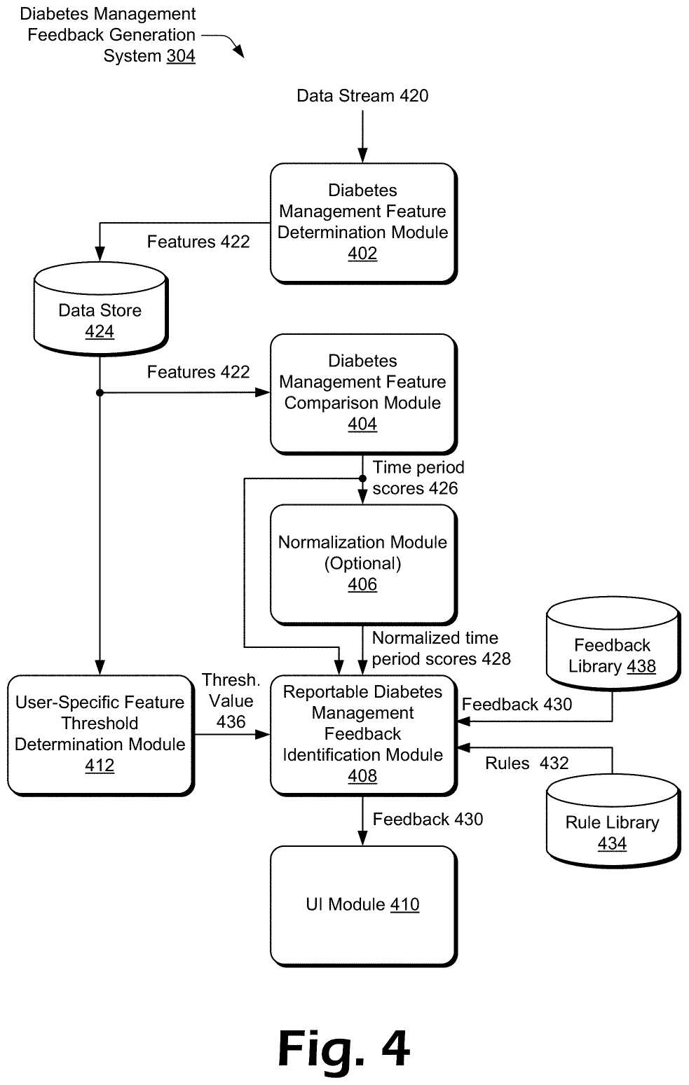

FIG. 4 is an illustration of an example architecture of a diabetes management feedback generation system 304. The diabetes management feedback generation system 304 includes a diabetes management feature determination module 402, a diabetes management feature comparison module 404, a normalization module 406 (optional), a diabetes management feedback identification module 408, a UI module 410 (optional), and a user-specific diabetes management feature threshold determination module 412. Generally, the diabetes management feedback generation system 304 analyzes the glucose measurements 114 for the user 102 and looks for patterns in the glucose measurements 114 that indicate good diabetes management by the user. Good diabetes management can be quantified in various manners, such as glucose measurements 114 staying within a particular range, glucose measurements 114 not varying by greater than a particular amount, and so forth. In one or more implementations, the diabetes management feedback generation system 304 identifies good diabetes management by identifying improvements in glucose measurements 114 for a given time period over one or more previous days, identifying a time period of the day during which glucose measurements 114 were the best (e.g., within an optimal range or closest to an optimal value), identifying sustained positive patterns (e.g., good diabetes management for a same time period in each of multiple days), or combinations thereof

The diabetes management feature determination module 402 receives a data stream 420 (e.g., the glucose measurements 114 and the additional data 302 of FIG. 3). In one or more implementations, the data stream 420 includes glucose measurements 114 and timestamps indicating when each of the glucose measurements 114 was taken (e.g., by wearable glucose monitoring device 104) or received (e.g., by glucose monitoring application 116). The timestamp may be provided, for example, by the wearable glucose monitoring device 104 or the glucose monitoring application 116. Additionally or alternatively, the data stream 420 includes additional data that may be used to identify good diabetes management (e.g., other data that affects glucose levels in the user 102). This additional data may also be referred to as additional data streams (e.g., the diabetes management feature determination module 402 receives multiple data streams 420 each including data from a different source or sensor).

For example, data stream 420 can include activity data, such as a number of steps walked over a particular range of time (e.g., every 10 seconds, every minute), heart rate over a particular range of time (e.g., at regular or irregular intervals, such as every 15 seconds) with timestamps, speed of movement with timestamp (e.g., at regular or irregular intervals, such as every 15 seconds), and so forth. Activity data can be received from various sources, such as wearable glucose monitoring device 104, an activity tracking application running on computing device 106, an activity or fitness tracker worn by the user 102, and so forth.

By way of another example, data stream 420 can include data regarding sleeping patterns of the user. E.g., data stream 420 can include data indicating times when the user is sleeping, the sleep state (e.g., Stage 1, Stage 2, Stage 3, or rapid eye movement (REM) sleep) of the user at particular times, and so forth.

By way of another example, data stream 420 can include data regarding user engagement with glucose monitoring application 116. E.g., this application engagement data can include timestamps of when the user 102 viewed the application as well as what screens or portions of the UI were viewed, timestamps of when the user 102 provided input to (or otherwise interacted with) the application 116 as well as what that input was, timestamps of when the user viewed or acknowledged feedback provided by diabetes management feedback generation system 304, and so forth.

By way of another example, data stream 420 can include data regarding user engagement with others of user population 108, such as via glucose monitoring platform 110. E.g., this other-user engagement data can include timestamps of when the user 102 communicated with another user as well as who that other user was, descriptions of what information was communicated with another user, and so forth.

By way of another example, data stream 420 can include meal data. E.g., this meal data can include timestamps of when the user 102 ate and what foods were consumed, timestamps of when particular types or classes of foods were consumed (e.g., vegetables, grain, meat, sweets, soda), amounts of food consumed, and so forth.

By way of another example, data stream 420 can include sleep data, such as data indicating minutes of the day when the user was sleeping. Sleep data can be received from various sources, such as wearable glucose monitoring device 104, a sleep tracking application running on computing device 106, an activity or fitness tracker worn by the user 102, and so forth.

By way of another example, data stream 420 can include medication data. E.g., this medication data can include timestamps of when user 102 took medicine and what medicine was taken (which can be used to determine whether the user 102 is taking his or her medicine at the prescribed times or intervals), indications of changes in medicines (e.g., changes in types or dosages of medicines taken), and so forth.

By way of another example, data streams 420 can include data that reflects stress management, such as heart rate variability (HRV), skin conductivity and temperature, respiration rate measurements, data from an electroencephalogram (EEG), cortisol in biofluids, volatile organic components (VOCs) emitted from the skin, and so forth.

By way of another example, data streams 420 can include data that relates to user interactions with the computing device 106, with display of the computing device 106, or with other system components that indicate level of engagement with diabetes management. Examples of such data include the number of times applications (e.g., glucose monitoring application) is opened, the time spent reviewing glucose data or previous insights or educational materials, the frequency of interactions with coaches or clinicians, and so forth.

In one or more embodiments, the diabetes management feedback generation system 304 receives data streams 420 including glucose measurements 114 and provides feedback for improving diabetes management. Furthermore, the diabetes management feedback generation system 304 optionally receives additional data in the data streams 420 that can be used to identify positive diabetes management behaviors or the impacts of these behaviors in the absence of glucose measurements 114. For example, if the user 102 only uses CGM periodically, but continues using glucose monitoring application 116 or another diabetes management application (or wears other devices that collect the additional data), the diabetes management feedback generation system 304 continues to provide feedback derived from this other data during the times when the CGM is not being used.

The diabetes management feature determination module 402 generates one or more features 422. A feature 422 refers to any value that can be computed from data in one or more data streams and that indicates whether the user has been engaging in beneficial diabetes management behaviors or lifestyle choices. A feature 422 can be a metric that is a representation or summarization of the data in the data stream 420 for a particular time period. In one or more implementations, each feature 422 is one or two values that represent or summarize the data in the data stream 420 for a particular time period, transforming the raw data obtained from the data stream 420 into a numeric indicator of the adherence to beneficial diabetes management and lifestyle choices. The diabetes management feature determination module 402 stores the generated features 422 in a data store 424 (e.g., maintained on storage device 118). The generated features 422 are maintained for a duration time that can vary by implementation. For example, the generated features 422 may be maintained for two weeks, one month, one year, and so forth.

In one or more implementations, each time period is a portion of a day (or other 24 hour interval). These time periods are chosen to capture the impacts of specific diabetes management decisions and lifestyle choices. In one or more implementations, each day is separated into multiple time periods based on when the user eats meals and when the user sleeps. For example, a day may include a first time period from midnight to 6 am (corresponding to sleep), a second time period from 6 am to noon (corresponding to after breakfast), a third time period from noon to 6 pm (corresponding to after lunch), and a fourth time period from 6 pm to midnight (corresponding to after dinner). Additionally or alternatively, additional time periods can correspond to other user actions that affect glucose levels, such as when the user exercises.

The glucose monitoring application 116 optionally provides a user interface via which the user 102 can customize the time periods to his or her typical schedule. For example, assume the user 102 typically goes to bed at 10 pm, eats breakfast at 7 am, eats lunch at noon, and eats dinner at 5 pm. These times can be provided to the glucose monitoring application 116 (e.g., by the user), which determines the time periods for the day to include a first time period from 10 pm to 7 am (corresponding to sleep), a second time period from 7 am to noon (corresponding to after breakfast), a third time period from noon to 5 pm (corresponding to after lunch), and a fourth time period from 5 pm to midnight (corresponding to after dinner). A day may be separated into other numbers of periods than four. For example, assume the user 102 typically goes to bed at 10 pm, exercises at 5 am, eats breakfast at 7 am, eats lunch at 11 am, eats an afternoon snack at 2 pm, and eats dinner at 6 pm. These times can be provided to the glucose monitoring application 116, which determines the time periods for the day to include a first time period from 10 pm to 5 am (corresponding to sleep), a second time period from 5 am to 7 am (corresponding to exercise), a third time period 7 am to 11 am (corresponding to after breakfast), a fourth time period from 11 am to 2 pm (corresponding to after lunch), a fourth time period from 2 pm to 6 pm (corresponding to snack), and a sixth time period from 6 pm to 10 pm (corresponding to after dinner).

Additionally or alternatively, different time periods for the user 102 can be automatically learned by the glucose monitoring application 116 by monitoring various data available to the glucose monitoring application 116 (e.g., exercise or sleep patterns from an activity tracker, eating patterns from a food or calorie tracking application) or detected directly (e.g., sleep onset detected by activity tracker). Various rules or criteria can be used to determine time periods based on the various data available to the glucose monitoring application 116, such as detecting sleep onset and sleep cessation from an activity tracker and using the times of sleep onset and sleep cessation to determine the time period corresponding to sleep.

In one or more implementations, the glucose monitoring application 116 uses a machine learning system to determine the different time periods for the user 102. Machine learning systems refer to a computer representation that can be tuned (e.g., trained) based on inputs to approximate unknown functions. In particular, machine learning systems can include a system that utilizes algorithms to learn from, and make predictions on, known data by analyzing the known data to learn to generate outputs that reflect patterns and attributes of the known data. For instance, a machine learning system can include decision trees, support vector machines, linear regression, logistic regression, Bayesian networks, random forest learning, dimensionality reduction algorithms, boosting algorithms, artificial neural networks, deep learning, and so forth.

The machine learning system is trained, for example, by using training data that is sets of multiple data (e.g., times of exercise, sleep, or eating during a day) and timestamps indicating when the exercise, sleep, or eating was done. Known labels are associated with the sets of multiple data indicating a time period that the data corresponds to. The machine learning system is trained by updating weights or values of layers in the machine learning system to minimize the loss between time periods generated by the machine learning system for the training data and the corresponding known labels for the training data. Various different loss functions can be used in training the machine learning system, such as cross entropy loss, mean squared error loss, and so forth.

In one or more implementations the machine learning system is trained over time as the glucose monitoring application 116 is used over time. E.g., the user can provide an indication of whether a particular time period is correct, and this indication can be used as a known label for the current time periods and used to further train the machine learning system.

Accordingly, different time periods can be established for different users. Furthermore, different time periods can be established for different days. For example, the user 102 may have different schedules on different types of days (e.g., a different schedule on weekends and holidays than on weekdays). Accordingly, the time periods for different types of days can be provided by the user 102 or determined by a machine learning system of the glucose monitoring application 116.

In one or more embodiments, the blocks of times for different time periods can vary for a user across different days. For example, a user may typically go to sleep between 11 pm and midnight, and wake up between 5:30 am and 6:30 am. For any given day, the time the user goes to sleep and the time the user wakes up can be detected using various data streams, such as data from an activity tracker worn by the user. Accordingly, the time period corresponding to sleep for the user may be 11:13 pm to 6:00 am for one day, 11:27 pm to 5:48 am the next day, 11:45 pm to 6:12 am the next day, and so forth.

In one or more embodiments, the time periods can vary for different data streams. The time periods for different data streams can be selected or identified with the intent of choosing a time window that best captures data related to the occurrence or characteristics of the behavior itself (e.g., meal choice), the impacts of the behavior on physiology (e.g., glucose, sleep quality, etc.), which may be delayed relative to the behavior, or a combination of the behavior and its impacts.

The diabetes management feature determination module 402 can generate, for each time period, any of a variety of features 422 for the data stream 420 and can generate different features 422 for different types of data in the data stream 420 (e.g., different features 422 for glucose measurements than for activity). For example, for glucose measurements the diabetes management feature determination module 402 can generate a time in range feature, such as an amount of time during the time period the glucose measurements were in an acceptable or desired range of glucose levels, e.g., between 70 milligrams per deciliter (mg/dL) and 180 mg/dL, or a narrow range between 70 mg/dL and 140 mg/dL. This acceptable or desired range can be a default range, can be a custom range set by the user or a health care professional, and so forth. By way of another example, for glucose measurements the diabetes management feature determination module 402 can generate a time below threshold feature, such as an amount of time during the time period the glucose measurements were below a particular glucose level (e.g., 250 mg/dL or 70 mg/dL). This particular glucose level can be a default level, can be a custom level set by the user or a health care professional, and so forth. By way of another example, for glucose measurements the diabetes management feature determination module 402 can generate a time above threshold feature, such as an amount of time during the time period the glucose measurements were above a particular glucose level (e.g., 250 mg/dL). This particular glucose level can be a default level, can be a custom level set by the user or a health care professional, and so forth. By way of another example, for glucose measurements the diabetes management feature determination module 402 can generate any of a variety of statistics, such as coefficient of variation for the glucose measurements in the time period (the ratio of the standard deviation to the mean for the glucose measurements in the time period), mean glucose measurement in the time period, standard deviation of the glucose measurements in the time period, and so forth. By way of another example, for glucose measurements the diabetes management feature determination module 402 can generate any of a variety of additional values, such as maximum glucose measurement in the time period, maximum glucose measurement rate of change in the time period, maximum glucose measurement rise in the time period, low blood glucose index (LBGI) in the time period, high blood glucose index (HBGI) in the time period, and so forth. By way of another example, for glucose measurements the diabetes management feature determination module 402 can generate a value indicating a rate of increase or decrease in glucose levels (e.g., a rapid rise around the time of an expected meal may allow the diabetes management feedback generation system 304 to infer that the patient consumed food with a high glycemic index, or a rapid drop from high glucose level associated with detected physical activity may allow the diabetes management feedback generation system 304 to infer that the user took action to reduce their glucose with exercise).

By way of another example, for activity data the diabetes management feature determination module 402 can generate a number of steps feature that is the number of steps taken during the time period, a heart rate reserve average or range during the time period, metabolic equivalents (METs) expended during the time period, and so forth. By way of another example, for activity data the diabetes management feature determination module 402 can generate a time in range feature, such as an amount of time during the time period the user's heart rate was in an acceptable or desired range of heart rates, e.g., between 304 beats per minute (BPM) and 170 BPM. This acceptable or desired range can be a default range, can be a custom range set by the user or a health care professional, and so forth. By way of another example, for activity data the diabetes management feature determination module 402 can generate a time above threshold feature, such as an amount of time during the time period the user's heart rate was above a particular level (e.g., 304 BPM). This particular level can be a default level, can be a custom level set by the user or a health care professional, and so forth.

By way of another example, for sleep data the diabetes management feature determination module 402 can generate a value indicating duration of sleep, number of sleep disturbances or interruptions, time spent in specific sleep states, and so forth.

The diabetes management feature comparison module 404 receives the different features 422 from the data store 424 (additionally or alternatively the features 422 may be received directly from the diabetes management feature determination module 402) and generates time period scores 426. The time period scores 426 indicate differences between the different features 422 generated for different time periods. The diabetes management feature determination module 402 generates the different features 422 for each time period in a day as discussed above. The time period scores 426 allow the diabetes management feature determination module 402 to compare the features 422 for different time periods within the same day, for the same time periods across different days or across different types of days, and so forth.

In one or more implementations, the diabetes management feature comparison module 404 compares the features 422 for each time period in a day to one another. Additionally or alternatively, the diabetes management feature comparison module 404 compares the features 422 for one or more time periods in a current day to the corresponding time periods in one or more previous days (e.g., the previous immediate week or two). The “current day” refers to a day (or other 24 hour interval) that the diabetes management feedback generation system 304 is analyzing. The current day can be, for example, the day the user 102 is currently living in, the day immediately preceding the day the user 102 is currently living in or another day in the user's past. In situations in which there are multiple types of days (e.g., weekends and holidays as one type and weekdays as another type), the diabetes management feature comparison module 404 compares the features 422 for one or more time periods in a current day (e.g., the day the user 102 is currently living in, or the immediately preceding day) to the corresponding time periods in one or more previous days of the same type (e.g., the previous immediate week or two for weekdays, the previous immediate three or four weeks for weekends and holidays).

Additionally or alternatively, the diabetes management feature comparison module 404 compares summary statistics of the features 422 computed from corresponding time periods on a set of previous days. For example, the diabetes management feature comparison module 404 can compare mean, median, interquartile range (IQR), XXth percentile, standard deviation, and so forth of features 422 computed from corresponding time periods on a set of previous days.

Additionally or alternatively, the diabetes management feature comparison module 404 compares the features 422 for one or more time periods in days of a week to the corresponding time periods in days of one or more preceding weeks. For example, for a particular diabetes management feature or feature (e.g., time in range), the feature value corresponding to a particular within-day time window (e.g., after breakfast) from a full week of data (the collection of after breakfast time windows from that week) could be compared to the same feature value for corresponding within-day time windows computed over a historical date range (e.g., the prior week or the preceding month).

In one or more implementations, the score for a time period is based on an effect size for the time period, a significance for the time period, the novelty of a generated feedback for the time period, or a combination thereof Additionally or alternatively, if there is variable duration time frame involved in the feedback, (e.g., the number of days over which a consistent pattern of positive glycemic control is detected), where the duration of the time frame indicates the relative “noteworthiness” of the feedback, this duration (or a normalized version of this duration) can serve as a score or as one component of a composite score for the time period.

The effect size for a time period refers to the difference between one time period and the other time period(s) that the one time period is being compared to. The effect size is computed in various manners, such as subtracting the features for one time period from the other or by comparing a feature from one time period to a summary statistic computed over a set of other time periods (e.g., comparing time in range after breakfast on the current day to the 90th percentile of time in range values computed for the after breakfast time period on each of the previous 14 days). For example, for a time in range feature for glucose measurements, the effect size can be computed by subtracting the amount of time in range for one time period from the amount of time in range for the other time period. E.g., if the time in range for a time period in the previous day is 304 minutes and the time in range for the corresponding time period on the current day is 150 minutes, then the effect size is 30 minutes. Additionally or alternatively, the effect size can be calculated as a percentage improvement. E.g., if the time in range for a time period in the previous day is 304 minutes and the time in range for the corresponding time period on the current day is 150 minutes, then the effect size is 25% due to 150 minutes being 25% greater than 304 minutes. Additionally or alternatively, the time in range can be identified as a percentage of the time period rather than a number of minutes. E.g., the time in range for a time period in the previous day may be 60% and the time in range for the corresponding time period on the current day may be 70%, so the effect size is 10%.

The significance of a time period refers to the difference between the one time period and the other time period(s) that the one time period is being compared to and accounts for the typical day-to-day variability for the user 102. This allows the diabetes management feedback generation system 304 to customize feedback to the typical behavior of user 102, as well as changes in the typical behavior of user 102 over time, rather than applying common thresholds or rules across all users. For example, one user may be pretty consistent with their behavior resulting in fairly consistent glucose levels, whereas another user may not be as consistent with their behavior resulting in wide swings in glucose levels. Accordingly, a smaller change in a feature would be more significant for a user that is consistent with their behavior than it would to a user that is not consistent with their behavior. The significance of the comparison accounts for these different behaviors.

The significance of a comparison can be generated in any of a variety of manners. For example, the significance of a comparison may be or include a value indicating, for a feature generated for a time period, a number of standard deviations away from the feature mean that generated feature is (e.g., the mean being calculated based on the features for a time period across multiple days, such as two weeks). Thus, the size of the standard deviation can vary on a user by user basis or over time for a given user (e.g., computed over a sliding window). Larger numbers of standard deviations away from the mean indicate a more significant difference.

The novelty of a generated feedback for the time period refers to how often or frequently a particular feedback is generated for the time period. For example, if feedback is frequently provided indicating that the time period corresponding to the breakfast time period is the best glucose levels of the day (e.g., the time period having glucose levels within an optimal range or closest to an optimal value), then the novelty score of the breakfast time period in the current day can be lower to avoid repeatedly providing the same feedback (which would be expected to be less interesting to the user). The novelty of a generated feedback for the time period is measured in any of various different manners, such as a count of how many times the feedback has been generated (e.g., over the previous two weeks or the previous month), a value indicating how frequently the feedback is generated relative to other feedbacks, and so forth.

The time period scores 426 can be output as any of a variety of different values. In one or more implementations, the time period score 426 for a time period is the effect size as determined by diabetes management feature comparison module 404. Additionally or alternatively, the time period score 426 for a time period can be a tuple including one or more of the effect size, the significance, the novelty (for each possible feedback), other indications of the differences between the different features, and so forth. Additionally or alternatively, the time period score 426 for a time period can be a value determined by combining (e.g., a weighted averaging) of one or more of the effect size, the significance, the novelty, other indications of the differences between the different features, and so forth.

In such situations, a different time period score 426 can be generated for each possible feedback (e.g., as different feedbacks could have different novelties).

In one or more implementations, the time period scores 426 are provided to a normalization module 406, which adjusts the time period scores 426 resulting from different scores (e.g., the effect size, the significance, and the novelty) to a common scale or common units (e.g., a value ranging between 0 and 1). The normalization module 406 outputs the normalized data as normalized time period scores 428. This normalization can be performed using any of a variety of public or proprietary techniques. It should be noted that the normalization module 406 is optional and that normalization need not be performed in certain situations. For example, in some situations if only a single type of score (e.g., one of effect size, significance, or novelty) is used by the diabetes management feedback generation system 304 there is no need to adjust time period scores 426 to a common scale or units (although time period scores 426 may still be adjusted to a common scale or units if the effect size is computed in terms of one of several possible diabetes management features, each feature having potentially different units). By way of another example, if multiple types of scores (e.g., two or more of effect size, significance, or novelty) are used by the diabetes management feedback generation system 304 but already have a common scale or units then there is no need to adjust time period scores 426 to a common scale or units.

The diabetes management feedback identification module 408 receives time period scores, which are the time period scores 426 or the normalized time period scores 428, and selects feedback 430 to be provided to UI module 410 or to the feedback presentation system 122 as feedback indications 312. The diabetes management feedback identification module 408 applies any of various different rules 432 from a rule library 434 to determine which of multiple feedback items from a feedback library 438 to retrieve and provide to the UI module 410 or the feedback presentation system 122. In one or more implementations, the feedback library 438 is the feedback library 124 of FIG. 1. The rule library and the feedback library 438 may be stored in various locations, such as stored in the storage device 118.

Various different feedback items can be included in the feedback library 438 and provided as feedback 430, each providing feedback to the user 102. Examples of such feedback include indicating improvement of glucose levels for a time period on the current day relative to previous days, a best time period of the day (e.g., the time period of the day where glucose levels were within an optimal range or closest to an optimal value (e.g., based on any one or more of the features discussed above)), feedback identifying sustained positive patterns (e.g., a feature being satisfied for the same time period across multiple days), and so forth.

In one or more implementations, the feedback in feedback library 438 is general feedback that need not be altered based on the specific features or glucose values of the user (e.g., a message such as “Your glucose levels were best after breakfast today!”). Additionally or alternatively, the feedback in feedback library 438 includes templates to which the diabetes management feedback identification module 408 adds features or glucose values to. For example, the feedback library 438 may include a message such as “You stayed in range for the night in a row!” where the underscore is filled in by the diabetes management feedback identification module 408. E.g., if the diabetes management feedback identification module 408 determines that the user was within the desired range for the three time periods corresponding to sleep, the diabetes management feedback identification module 408 replaces the blank space with “3^{rd}”.

Additional examples of feedback include: “Overnight glucose 70-140 mg/dL>40% of time for 5 days in a row,” “After-lunch peak glucose<130 mg/dL for 3 days in a row,” “After-lunch peak glucose22 mg/dL lower than any of the past 7 days,” “18% more time 70-140 mg/dL after dinner than any of the last 7 days,” “5% more time 70-180 mg/dL after dinner than any other time period today,” “Your peak glucose after lunch was 14 mg/dL lower than any other time period today,” “Your average glucose after dinner today was 21 mg/dL lower than any of the last 7 days,” “Your time between 70 and 140 mg/dL after dinner was 4% higher than any other time period today,” and “Your time between 70 and 140 mg/dL after lunch today was 3% higher than any of the last 7 days.”

Various different rules 432 can be included in the rule library 434, and each rule 432 corresponds to one of the items of feedback in feedback library 438. Rules can be directed to different features and different time periods within a day or corresponding time periods across multiple days. For example, one rule can be which time period of the day has a highest score (e.g., the glucose measurements for a particular time period of the day were within a particular range or were below a particular glucose level for a longer duration of time than the glucose measurements during the other time periods of the day). Another rule can be whether glucose measurements for a particular time period of the day improved a threshold amount over the glucose measures for the corresponding time periods of one or more previous days (e.g., the glucose measurements were within a particular range or were below a particular glucose level for at least a threshold longer duration of time than the glucose measurements measured for the user during the time period of the one or more previous days). Another rule can be whether glucose measurements for a particular time period of the day were part of a sustained pattern of good glucose measurements (e.g., the glucose measurements for the current day as well as glucose measurements measured for the time period of one or more previous days (e.g., two to four weeks) were within a particular range or were below a particular glucose level for each of the current day and the one or more previous days).

The rules 432 can include reference to various thresholds, such as threshold values being exceeded or not being exceeded. In one or more implementations, the user-specific diabetes management feature threshold determination module 412 generates a threshold value 436, based on the features 422, that is a user-specific diabetes management feature threshold based on the distribution of the diabetes management feature observed over a historical time window (e.g., the threshold may be set to the 75th percentile of the diabetes management feature observed in a given within-day time period over the prior month) for the user 102. This threshold value 436 is then used by the diabetes management feedback identification module 408 as the threshold value in various rules 432 (for example, to identify sequences of days that meet or exceed that threshold (e.g., the sustained positive pattern)). Deriving the threshold value 436 from a user's own historical data allows the threshold value 436 to represent a level of glycemic control that represents glycemic control that is “good” relative to the typical levels observed for that user, but still achievable. The choice of summary statistic to use to set the threshold (e.g., 60th percentile versus 80th percentile) is a parameter that allows control over the balance between the “noteworthiness” of the sustained positive pattern and the achievability for that particular patient. The choice of summary statistic to use to set the threshold may be made by the user 102, by a health care professional, by an administrator or designer of the glucose monitoring application 116 or the glucose monitoring platform 110, and so forth. The threshold value 436 can also adapt to within-user changes in glycemic control over long time periods because it can be updated as the historical time window used to derive the threshold value 436 advances in time.

In one or more implementations, the diabetes management feedback identification module 408 uses a rule 432 indicating that if the score corresponding to a feature for a time period exceeds a threshold, then the rule is satisfied and feedback corresponding to that rule is selected as feedback 430. This can result in diabetes management feedback identification module 408 selecting multiple items of feedback 430 that are displayed or otherwise presented by UI module 410 or provided to feedback presentation system 122.

Additionally or alternatively, in situations in which for multiple rules are satisfied, diabetes management feedback identification module 408 selects one of the rules that was satisfied and selects as feedback 430 the feedback corresponding to the selected rule. The diabetes management feedback identification module 408 can select one of the multiple rules in various manners, such as randomly or pseudorandomly selecting one of the multiple rules that were satisfied. Additionally or alternatively, the diabetes management feedback identification module 408 can prioritize the multiple rules and select one of the multiple rules having a highest priority. For example, the rule having the highest priority is selected.

The diabetes management feedback identification module 408 optionally uses various criteria to determine which of the multiple rules that were satisfied to select. These criteria can be based on various factors, such as how recently a rule was satisfied, a ranking or prioritization of rules, categories of rules, how many consecutive days the rule has been satisfied, and so forth. For example, a rule that was satisfied less recently is selected over a rule that was satisfied more recently. E.g., this allows different feedback (corresponding to the different rules) to be selected as feedback 430 and avoids repeating feedback too frequently.

By way of another example, rules may correspond to different categories, such as an improvement category (e.g., rules corresponding to improvements in glucose measurements for a time period over one or more previous days), a best-of category (e.g., rules identifying a time period of the day during which glucose measurements were the best (e.g., within an optimal range or closest to an optimal value)), a sustained pattern category (e.g., rules identifying sustained positive patterns), and so forth. Rules corresponding to certain categories can be selected over rules corresponding to other categories. For example, a rule corresponding to a sustained pattern category may be selected over a rule corresponding to an improvement category, e.g., to avoid displaying feedback repeating the same improvement too frequently.

By way of another example, a rule designated (e.g., by a developer or designer of the diabetes management feedback identification module 408) to be more urgent or safety-related is selected over a rule that is less urgent or safety-related. E.g., this allows feedback corresponding to urgent or safety-related features (e.g., not staying within ranges or exceeding threshold glucose levels) to be selected over other non-urgent or non-safety-related features and display or otherwise present more critical diabetes management feedback to the user.

By way of another example, a rule designated as being higher priority (e.g., by the user 102) is selected over a rule that is designated as being lower priority (e.g., by the user 102). E.g., this allows feedback corresponding to rules that are of greater interest to the user to be displayed or otherwise presented rather than feedback corresponding to rules that are of less interest to the user.

By way of another example, a rule that has been satisfied for at least a threshold number of days in a row is selected over a rule that has not been satisfied for at least the threshold number of days in a row. E.g., this allows a good streak or good pattern (e.g., peak glucose staying below 200 mg/dL during an after lunch time period) for a number of days to be displayed or otherwise presented to the user, encouraging the user to continue with the streak or pattern.

In one or more implementations, feedback 430 is generic in nature, notifying the user 102 of particular time periods for which glucose management was good without specifying particular values indicating an amount of improvement (e.g., indicating “Your after-dinner glucose is looking great!” rather than “Your after-dinner glucose was lower than usual today”). In such situations, the diabetes management feedback identification module 408 selects feedback 430 corresponding to the time period for which multiple rules were satisfied. For example, assume that for a current day a first time period and a second time period both satisfied a time in range rule, the first time period did not satisfy a coefficient of variation rule for the glucose measurements in the time period, but the second time period did satisfy the coefficient of variation rule for the glucose measurements in the time period. In this situation, the diabetes management feedback identification module 408 selects feedback 430 corresponding to the second time period rather than the first time period because there were more rules satisfied by the second time period than the third time period. By way of another example, assume that for a current day a first time period and a second time period both satisfied a time in range rule, the first time period did not satisfy a number of steps taken rule during the time period, but the second time period did satisfy a number of steps taken rule during the time period. In this situation, the diabetes management feedback identification module 408 selects feedback 430 corresponding to the second time period rather than the first time period because there were more rules satisfied by the second time period than the third time period.

In one or more implementations, the UI module 410 receives the feedback 430 and causes the feedback 430 to be displayed or otherwise presented (e.g., at computing device 106). This display or other presentation can take various forms, such as a static text display, graphic or video display, audio presentation, combinations thereof, and so forth. In one or more implementations, different types or categories of feedback are displayed or otherwise presented in different manners. For example, feedback categories can include a category of improvements in glucose measurements for a given time period over one or more previous days, a category of which time period of the day had the best glucose measurements (e.g., which time period had glucose measurements within an optimal range or closest to an optimal value), and a category of sustained positive patterns. Feedback corresponding to these different categories can be displayed using different colors, different icons, and so forth.

In one or more implementations, the feedback 430 is ordered based on priority. For example, if multiple rules were satisfied the diabetes management feedback identification module 408 selects the highest priority feedback first as feedback 430. User input requesting additional feedback of lower priority as desired by the user 102 can be received, and the diabetes management feedback identification module 408 provides the lower priority feedback as requested by the user. E.g., this allows the highest priority feedback to be presented, then in response to a user request for additional feedback the second highest priority feedback to be presented, then in response to an additional user request for additional feedback the third highest priority feedback to be presented, and so forth.

In one or more implementations, the diabetes management feedback identification module 408 determines whether there is an improvement, based on at least one feature, in the glucose levels of the user for each of the time periods in the current day relative to one or more previous days, such as the immediately preceding week or two (these previous days are optionally days of the same type). If the diabetes management feedback identification module 408 determines that a rule indicating that the improvement for a time period satisfies a threshold (e.g., at least a certain percentage improvement), the diabetes management feedback identification module 408 selects feedback 430 corresponding to the rule.

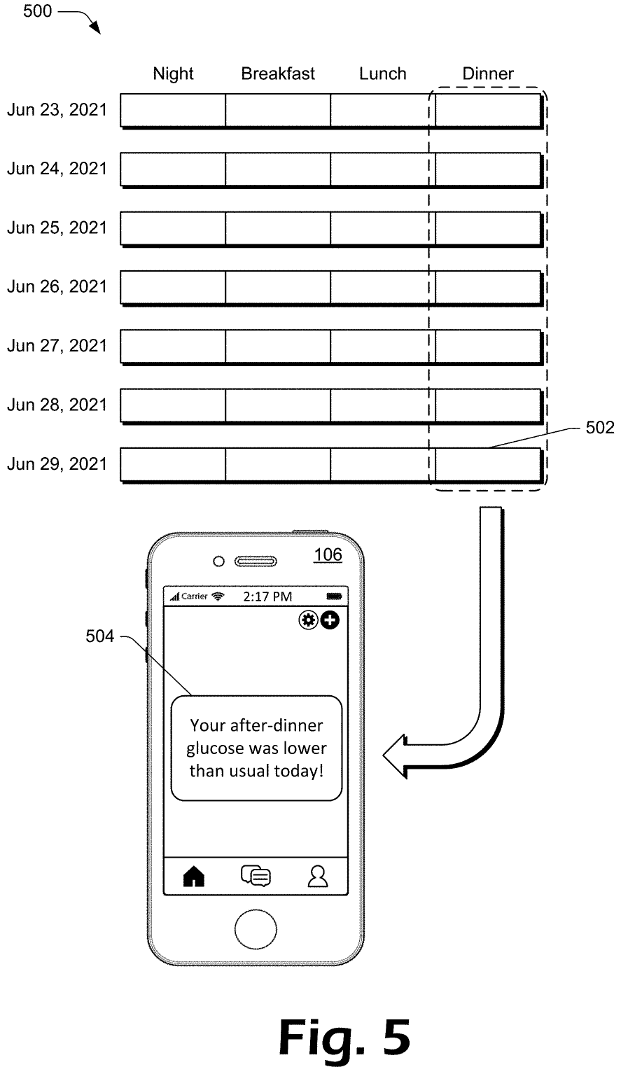

FIG. 5 illustrates an example 500 of providing feedback indicating improvement in at least one feature for a time period. The improvement in example 500 is an improvement in a time period of a current day relative to the corresponding time period in each of multiple immediately preceding days. The example 500 show multiple days (illustrated as Jun. 23, 2021-Jun. 29, 2021) each having time periods Night (e.g., while the user is sleeping), Breakfast (e.g., during and after breakfast), Lunch (e.g., during and after lunch), and Dinner (e.g., during and after dinner). A time period score is generated for time period 502 (Dinner) on the current day (June 29) that indicates the difference between one or more features for time period 502 and the same one or more features for the corresponding time period (Dinner) in the previous days June 23-June 28. In the illustrated example, the comparisons for these time periods and generated score indicate that the user's glucose level during time period 502 was lower than the typical amount for the user during the previous days June 23-June 28, which satisfies a rule 432. This determination can have been made in various manners, such as determining how many standard deviations the glucose level for time period 502 on June 29 was away from the mean generated for the corresponding time period on June 23-June 28.

The diabetes management feedback identification module 408 identifies feedback 504 and the UI module 410 (or the feedback presentation system 122) causes the feedback 504 to be displayed. In the illustrated example, the feedback 504 is an inspirational insight notifying the user that his after-dinner glucose was lower than usual today. This brings the improved glucose level for a time period to the user's attention, allowing him to reflect on what changes he made to his dinner on June 29 relative to the previous days and continue those changes for better diabetes management.

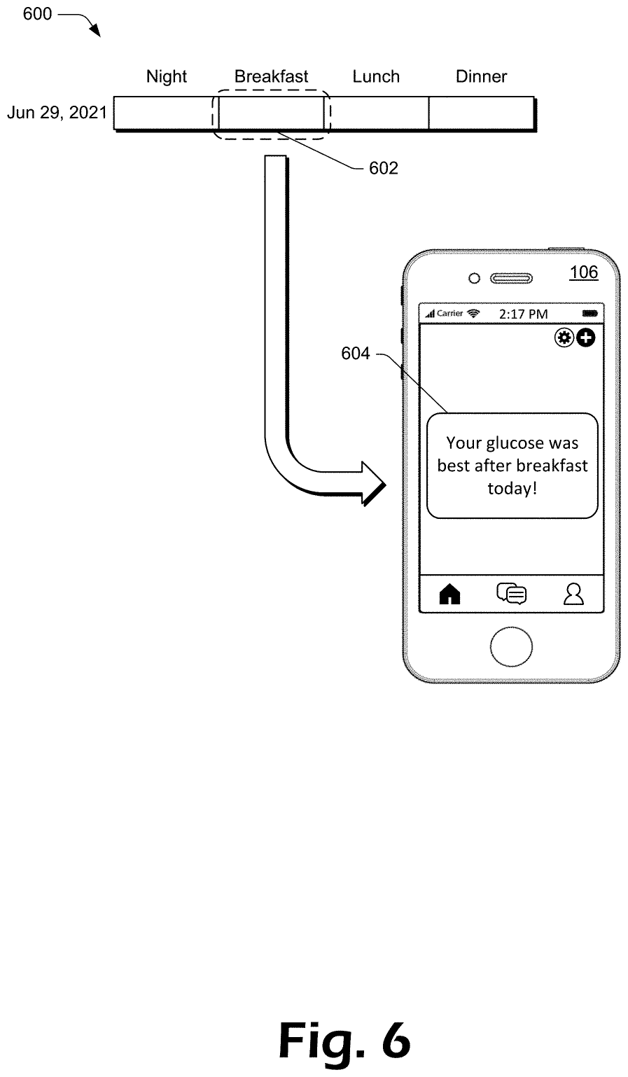

FIG. 6 illustrates an example 600 of providing feedback indicating a best time period during a day. The example 600 show a single day (illustrated as Jun. 29, 2021) having time periods Night (e.g., while the user is sleeping), Breakfast (e.g., during and after breakfast), Lunch (e.g., during and after lunch), and Dinner (e.g., during and after dinner). A time period score is generated for time period 602 (Breakfast) on the current day (June 29) that indicates the difference between one or more features for time period 602 and the same one or more features for the other three time periods on June 29. In the illustrated example, the comparisons for these time periods and generated score indicate that the user's glucose level during time period 602 was better than any other time periods on June 29, which satisfies a rule 432. This determination can have been made in various manners, such as determining that the user's glucose level was within a particular range longer than during any other time period on June 29, determining that the user's glucose level remained below a threshold level during time period 602 but not during other time periods on June 29, and so forth.

The diabetes management feedback identification module 408 identifies feedback 604 and the UI module 410 (or the feedback presentation system 122) causes the feedback 604 to be displayed. In the illustrated example, the feedback 604 is an inspirational insight notifying the user that his after-breakfast glucose was better than any other time period today. This brings the improved glucose level for a time period to the user's attention, allowing him to reflect on what foods he eats during breakfast relative to the foods eaten during other time periods on June 29, and using that knowledge to change foods eaten during other time periods for better diabetes management.

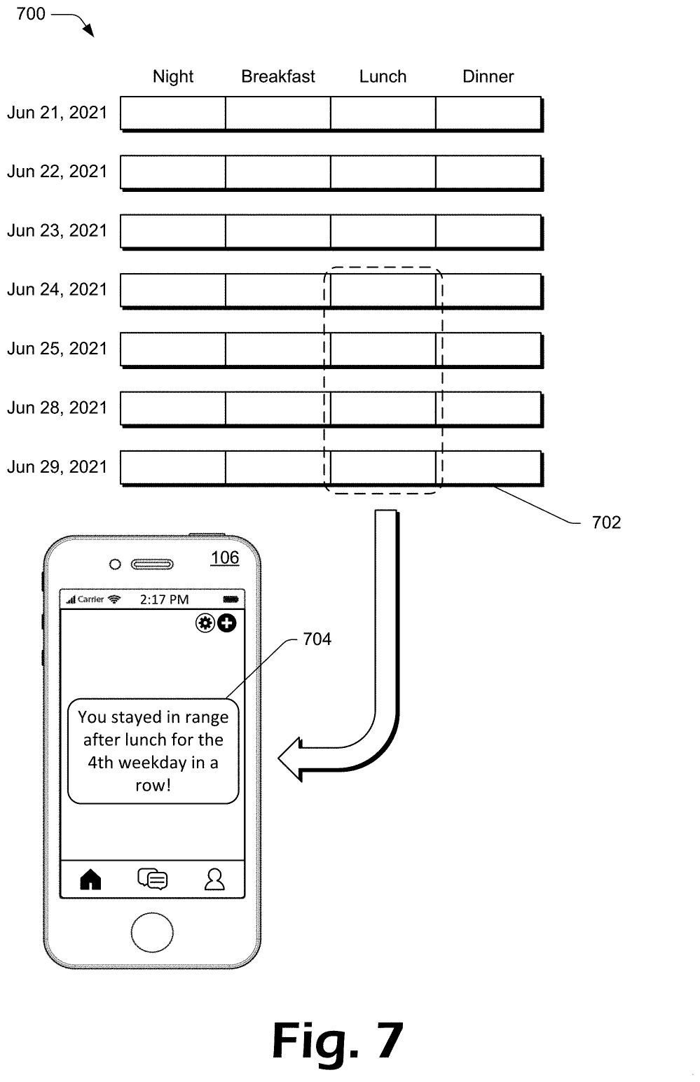

FIG. 7 illustrates an example 700 of providing feedback indicating a sustained positive pattern. The sustained positive pattern in example 700 is a pattern in a corresponding time period across multiple consecutive days of the same type (e.g., weekdays). The example 700 shows multiple days (illustrated as Jun. 21, 2021-Jun. 25, 2021, Jun. 28, 2021, and Jun. 29, 2021) each having time periods Night (e.g., while the user is sleeping), Breakfast (e.g., during and after breakfast), Lunch (e.g., during and after lunch), and Dinner (e.g., during and after dinner). A time period score is generated for time period 702 (Lunch) on the current day (June 29) that indicates the difference between one or more features for time period 702 and the same one or more features for the corresponding time period (Lunch) in each of the previous days June 21-June 25 and June 28. In the illustrated example, the comparisons for these time periods and generated score indicate that the user's glucose level during time period 702, as well as the corresponding time period on June 24, June 25, and June 28, was within a particular range, which satisfies a rule.

The diabetes management feedback identification module 408 identifies feedback 704 and the UI module 410 (or the feedback presentation system 122) causes the feedback 704 to be displayed. In the illustrated example, the feedback 704 is an inspirational insight notifying the user that his overnight glucose was within the desired range after lunch for 4 weekdays in a row. This brings the sustained pattern of staying within the desired range to the user's attention, allowing him to reflect on what types of food he has been eating for lunch over the past few weekdays and continue to eat similar types of food for better diabetes management.

Returning to FIG. 4, discussions are made herein with reference to multiple time periods in a time range that is a day. Additionally or alternatively, different time periods or time ranges can be used. For example, each time period can be an entire day and the time range can be a week, a month, multiple months, a year, and so forth. By way of another example, each time period can be an entire week and the time range can be a month, multiple months, or an entire year.

Discussions are also made herein with reference to positive comparisons indicating good diabetes management and displaying or otherwise presenting feedback (e.g., inspirational insights) to motivate the user. Analogously, negative comparisons indicating poor diabetes management can be made and feedback (e.g., warnings or negative affects) displayed or otherwise presented to the user to encourage better diabetes management by the user. Negative comparisons are analogous to the techniques discussed herein, but the rules applied to the features are different. For example, feedback indicating a time period having the worst glucose levels of the day (e.g., a time period having glucose levels outside of an optimal range or furthest from an optimal value) may be displayed rather than a time period having the best glucose levels of the day (e.g., a time period having glucose levels within an optimal range or closest to an optimal value). By way of another example, feedback indicating that a time in range for a given time period was 25% less than corresponding periods in the previous days. Additional features can also be included for negative comparisons. For example, for glucose measurements the diabetes management feature determination module 402 can generate a threshold exceeded feature, such as whether a particular glucose level (e.g., 400 mg/dL) was exceeded during the time period.

In one or more implementations, the diabetes management feedback generation system 304 applies additional criteria to determining when to display or otherwise presenting negative feedback such as a warning or negative affects. This additional criterion can include identifying a pattern of the feature being satisfied on multiple occurrences, e.g., a particular time period being the worst glucose levels of the day for multiple days in a row, or the time in range for a given time period being 25% less than corresponding periods in the previous days for two days in a row. This additional criterion prevents a warning or negative affect from being displayed or otherwise presented to the user for a single bad day, avoiding unnecessarily warning the user or notifying him or her of a negative affect for a single aberration.

As discussed above, situations arise in which diabetes management feedback identification module 408 uses various criteria to determine which of multiple rules that were satisfied to select. In one or more implementations, negative comparisons are one of these criteria and operate to cause a rule corresponding to a time period that did not satisfy a negative comparison to be selected over a time period that did satisfy a negative comparison. For example, assume that for a current day a first time period and a second time period both satisfied a time in range rule, the first time period did not satisfy the negative comparison of the threshold exceeded rule (e.g., the threshold glucose level was not exceeded during the time period), but the second time period did satisfy the negative comparison of the threshold exceeded rule (e.g., the threshold glucose level was exceeded during the time period). In this situation, the diabetes management feedback identification module 408 selects a rule for the first time period rather than the second time period because there was no negative comparison satisfied for the first time period.

The diabetes management feedback generation system 304 optionally takes additional actions based on the feedback 430. In one or more implementations, these actions include notifying the glucose monitoring application 116 or the wearable glucose monitoring device 104 that the frequency with which glucose measurements 114 are produced can be reduced. For example, if the diabetes management feedback generation system 304 identifies a sustained positive pattern (e.g., glucose within a particular range) for a particular time period for at least a threshold number of days, the diabetes management feedback generation system 304 notifies the glucose monitoring application 116 or wearable glucose monitoring device 104 that the frequency with which glucose measurements 114 are produced can be reduced (e.g., from every 5 minutes to every 10 minutes), reducing the power expended to produce glucose measurements 114.

Additionally or alternatively, these actions include determining whether to recommend ongoing CGM use (e.g., starting a new sensor immediately after the current sensor expires) or whether it may be appropriate to take a break from using CGM and starting a new sensor at some later date. For example, if the diabetes management feedback generation system 304 identifies no sustained positive pattern (e.g., glucose within a particular range) for a particular time period for at least a threshold number of days, the diabetes management feedback generation system 304 recommends (e.g., via display or other presentation to the user) ongoing CGM use.

Discussions are also made herein with reference to feedback being displayed or otherwise presented to the user 102. Additionally or alternatively, the feedback is communicated to or otherwise delivered to others, such as a clinician (e.g., the user's primary care physician or nurse), a pharmacist, and so forth. This can serve to partially automate some of the manual effort of reviewing raw glucose or other diabetes management data that a clinician may have to do on their own in the absence of generated feedback. Additionally or alternatively, the time period scores 426 or normalized time period scores 428 may be provided to the clinician, pharmacist, or others, enabling them to apply their own preferred set of rules in determining which feedback should be passed along to the user.

It should be noted that, as discussed above, the feedback 430 can be provided to the feedback presentation system 122 as feedback indications 312. In such situations the diabetes management feedback generation system 304 need not include the UI module 410. Additionally or alternatively, the time period scores 426 (or normalized time period scores 428) and optionally the threshold value 436 can be provided to the feedback presentation system 122 as feedback indications 312. In such situations the feedback presentation system 122 identifies feedback to be provided to the user (or others, such as a clinician or pharmacist), optionally using the feedback library 438 and the rule library 434, as discussed in more detail below. The feedback presentation system 122 optionally identifies feedback to be provided to the user using any one or more of the techniques discussed herein with respect to the reportable diabetes management feedback identification module 408.

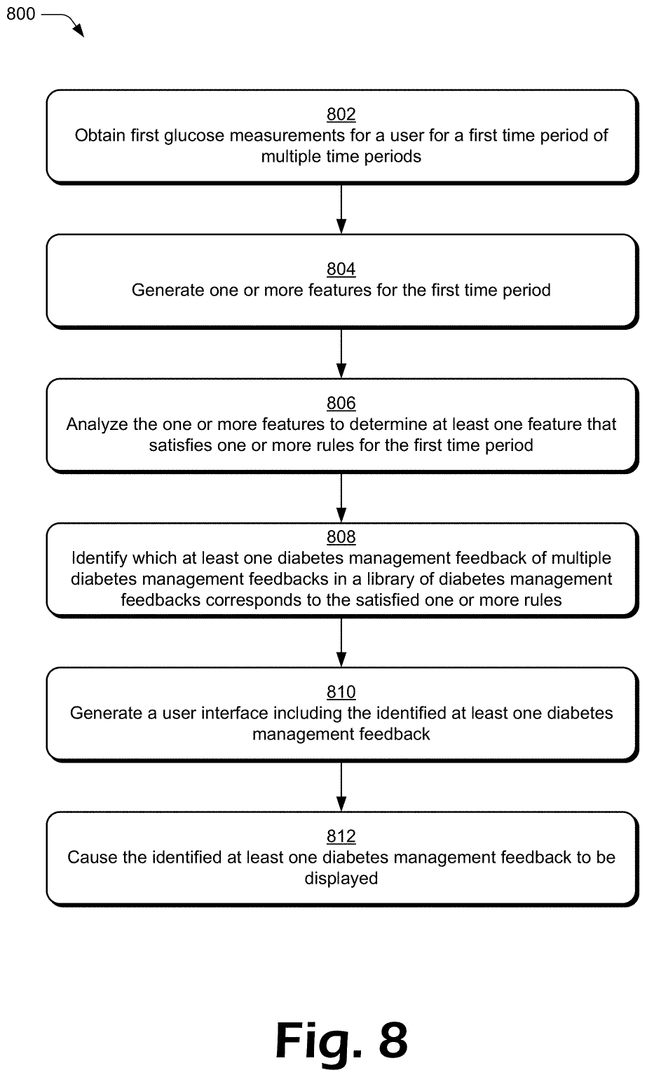

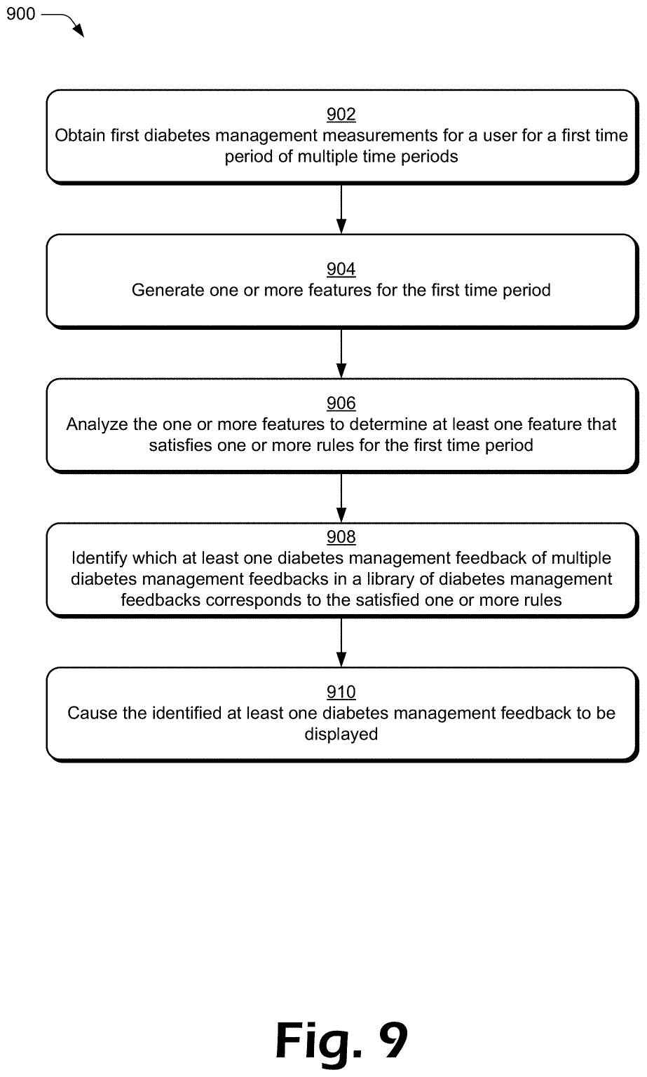

FIG. 8 and FIG. 9 describe examples of procedures for implementing diabetes management feedback for improving diabetes management. Aspects of the procedures may be implemented in hardware, firmware, or software, or a combination thereof The procedures are shown as a set of blocks that specify operations performed by one or more devices and are not necessarily limited to the orders shown for performing the operations by the respective blocks.

FIG. 8 depicts a procedure 800 in an example of implementing diabetes management feedback for improving diabetes management. Procedure 800 is performed, for example, by a diabetes management feedback generation system, such as the diabetes management feedback generation system 304 and optionally in part by a feedback presentation system, such as the feedback presentation system 122.

First glucose measurements for a user for a first time period of multiple time periods are obtained (block 802). These first glucose measurements are obtained from a glucose sensor of, for example, a continuous glucose level monitoring system with the glucose sensor being inserted at an insertion site of the user.

One or more features for the first time period are generated (block 804). These one or more features are generated from the first glucose measurements.

The one or more features are analyzed to determine at least one feature that satisfies one or more rules for the first time period (block 806). The analysis can involve different time periods in a same 24 hour interval as the first time period, or corresponding time periods across multiple 24 hour intervals.

Which at least one diabetes management feedback of multiple diabetes management feedbacks in a library of diabetes management feedbacks corresponds to the one or more rules is identified (block 808). Different rules can correspond to different feedback, and that feedback is identified in block 808. Additionally or alternatively, different time periods can correspond to different feedback, and the feedback corresponding to the first time period is identified in block 808.

A user interface including the at least one identified diabetes management feedback and an indication of the first time period is generated (block 810). The identified at least one diabetes management feedback is caused to be displayed (block 812) or otherwise presented.

FIG. 9 depicts a procedure 900 in another example of implementing diabetes management feedback for improving diabetes management. Procedure 900 is performed, for example, by a diabetes management feedback generation system, such as the diabetes management feedback generation system 304 and optionally in part by a feedback presentation system, such as the feedback presentation system 122.

First diabetes management measurements for a user for a first time period of multiple time periods are obtained (block 902). These first diabetes management measurements can be obtained from a glucose sensor of from any of a variety of other sensors as discussed above (e.g., any sensor providing data for data stream 420).

One or more features for the first time period are generated (block 904). These one or more features are generated from the first diabetes management measurements.

The one or more features are analyzed to determine at least one feature that satisfies one or more rules for the first time period (block 906). The analysis can involve different time periods in a same 24 hour interval as the first time period, or corresponding time periods across multiple 24 hour intervals.

Which at least one diabetes management feedback of multiple diabetes management feedbacks in a library of diabetes management feedbacks corresponds to the one or more rules is identified (block 908). Different rules can correspond to different feedback, and that feedback is identified in block 908. Additionally or alternatively, different time periods can correspond to different feedback, and the feedback corresponding to the first time period is identified in block 908.

The identified at least one diabetes management feedback is caused to be displayed (block 910) or otherwise presented. Additionally or alternatively, the identified diabetes management feedback can be communicated to or otherwise presented to a clinician, pharmacist, or other health care provider.

### Glucose Level Deviation Detection System Architecture

Generally, glucose level deviation detection system 306 receives a data stream of glucose measurements. Aggregate metrics (e.g., hyperglycemic risk values, hypoglycemic risk values, mean glucose, mean coefficient of variation, etc.) are generated for collections of glucose measurements, such as over rolling windows of time (e.g., every 5 minutes, a collection of glucose measurements includes the glucose measurements during the preceding 30 or 60 minutes), at fixed 30-minute intervals (e.g., on every hour and every half hour of the day), the preceding 60 minutes, and so forth. These aggregate metrics are compared to aggregate metrics generated in other time periods to identify deviations in glucose measurements between time periods.

For example, risk values may be generated for time periods that include glucose measurements received between particular fixed times (e.g., approximately every half hour of the day). The aggregate metrics for a given time period are compared to the aggregate metrics over a number (e.g., 24) of immediately preceding time periods in the same day to determine whether the aggregate metrics for the given time period deviate from the aggregate metrics of the preceding time periods. If a deviation is detected, an indication that there is a deviation between the glucose measurements measured during the given time period and the glucose measurements measured during the preceding time periods is displayed or otherwise communicated. E.g., a user interface reading “Your glucose is increasing to the highest level it has been since this morning” may be displayed to the user.

By way of another example, aggregate metrics may be generated for time periods that include glucose measurements received over a preceding amount of time (e.g., every 5 minutes aggregate metrics are generated based on glucose measurements received over the preceding 60 minutes). The aggregate metrics for a given time period on the current day are compared to the aggregate metrics generated for the corresponding time periods on each of multiple preceding days to determine whether the aggregate metrics for the given time period deviate from the aggregate metrics of the corresponding time periods on the multiple preceding days. If a deviation is detected (and optionally if the deviation is selected for display), an indication that there is a deviation between the glucose measurements measured during the given time period on the current day and the glucose measurements measured during the preceding time periods is displayed or otherwise communicated. E.g., a user interface reading “Your glucose over the last hour is higher than it has been at this time for each of the past few days” may be displayed to the user.

The techniques discussed herein automatically detect deviations in glucose levels for the user and provide notifications of such to the user. This makes the user aware of the deviations in real time as they occur, alerting the user to the potential that they are doing something different that is having a significant impact on their glucose levels. This provides teachable moments to the user, helping the user make a connection between real time events or actions and changes in glucose levels, and thus alter their behavior now and in the future so as to avoid such changes in glucose levels (e.g., if the changes are bad) or to maintain such changes in glucose levels (e.g., if the changes are good). Furthermore, this allows warnings or updates to be communicated to healthcare providers so that those providers can help the user take correction action, be alerted to the severity of changes, and so forth.

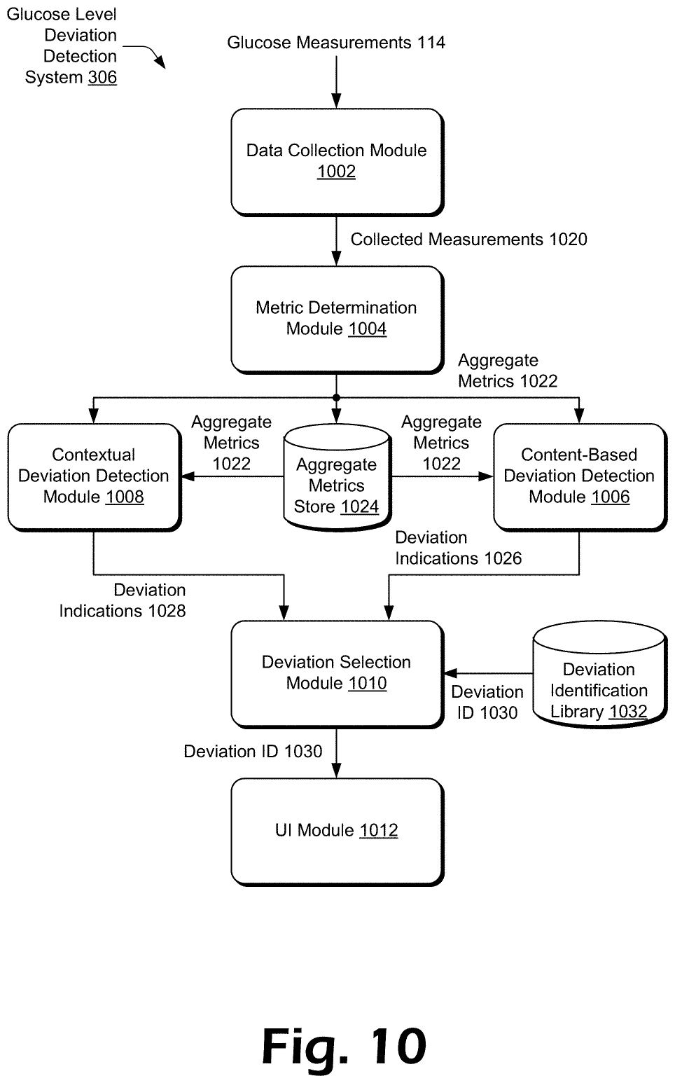

FIG. 10 is an illustration of an example architecture of a glucose level deviation detection system 306. The glucose level deviation detection system 306 includes a data collection module 1002, a metric determination module 1004, a content-based deviation detection module 1006, a contextual deviation detection module 1008, a deviation selection module 1010, and a UI module 1012 (optional). Generally, the glucose level deviation detection system 306 analyzes the glucose measurements 114 for the user 102 and looks for deviations from a norm for the user. These deviations from the norm can be based on various factors, such as metrics generated from the user's current or recent glucose level relative to metrics generated from the user's glucose levels earlier in the day, the metrics generated from the user's current or recent glucose level relative to metrics generated from the user's glucose levels in corresponding times of previous days, and so forth. Upon detection of one or more deviations, the glucose level deviation detection system 306 takes a responsive action, such as presenting an identification of the deviation to the user, communicating an identification of the deviation to a healthcare professional, including the deviation in the feedback indications 312, and so forth.

More specifically, the data collection module 1002 receives glucose measurements 114 for user 102. The data collection module 1002 optionally also receives the additional data 302 of FIG. 3. The glucose measurements 114 are received at a particular interval, such as approximately every 1 minute or approximately every 5 minutes. The glucose measurements 114 are grouped together into collections of measurements. In one or more implementations, the collections of measurements are set time periods within a 24-hour period, such as the glucose measurements 114 received during every half-hour time period (e.g., from 1:00 pm to 1:30 pm, from 1:30 pm to 2:00 pm, from 2:00 pm to 2:30 pm, and so forth). Additionally or alternatively, the collections of measurements are rolling windows of time, such as the glucose measurements 114 received over the previous 30 or 60 minutes. E.g., when a new glucose measurement 114 is received (such as approximately every 5 minutes), the data collection module 1002 groups the glucose measurements 114 received over the previous 30 minutes or 1 hour into a collection of measurements. The data collection module 1002 outputs the collections of measurements as collected measurements 1020.

The metric determination module 1004 receives the collected measurements 1020 and generates one or more aggregate metrics 1022 (or single-value metrics) from the collected measurements 1020. An aggregate metric (also referred to as simply a metric) is a representation or summarization of the data in the collected measurements 1020. The metric determination module 1004 can generate any of a variety of different metrics based on the collected measurements 1020. In one or more implementations, the metric determination module 1004 generates risk values that are glycemic risk values indicating a potential health risk to the user 102 due to glucose levels.

Additionally or alternatively, the metric determination module 1004 generates as aggregate metrics 1022 any of a variety of statistics from the collected measurements 1020, such as mean glucose measurement in the collected measurements 1020, mean coefficient of variation for the glucose measurements in the collected measurements 1020 (the ratio of the standard deviation to the mean for the glucose measurements in the collected measurements 1020), mean amplitude of glycemic excursions (MAGE) for the glucose measurements in the collected measurements 1020, the area under the glucose curve for the glucose measurements in the collected measurements 1020, the area above the glucose curve for the glucose measurements in the collected measurements 1020, the mean absolute rate of change for the glucose measurements in the collected measurements 1020, the standard deviation of the glucose measurements in the collected measurements 1020, the mean amount of time during which the collected measurements 1020 were collected that the glucose measurements were below a particular glucose level (e.g., 250 mg/dL or 70 mg/dL), the mean amount of time during which the collected measurements 1020 were collected that the glucose measurements were above a particular glucose level (e.g., 250 mg/dL), the maximum glucose measurement in the collected measurements 1020, and so forth.

Additionally or alternatively the metric determination module 1004 generates aggregate metrics 1022 using different techniques for combining glucose measurements in the collected measurements 1020, such as the median, the interquartile range (IQR), the XXth percentile, the standard deviation, and so forth.

Additionally or alternatively, the metric determination module 1004 generates single-value metrics that are not an aggregate metric. For example, the metric determination module 1004 can generate a metric that is a maximum glucose measurement in the collected measurements 1020, an absolute rate of change for the glucose measurements in the collected measurements 1020, and so forth. The metric determination module 1004 outputs these single-value metrics which are used by the content-based deviation detection module 1006 and the contextual deviation detection module 1008 analogous to the aggregate metrics 1022.

In one or more implementations, the aggregate metrics 1022 are glycemic risk values indicating a potential health risk to the user 102 due to glucose levels. In one or more embodiments, these risk values include one or both of a hyperglycemic risk value and a hypoglycemic risk value. The hyperglycemic and hypoglycemic risk values can be determined in any of a variety of different manners.

In one or more embodiments, the hyperglycemic risk value is based on a high blood glucose index (HBGI) value generated for self-monitoring blood glucose (SMBG) readings. The HBGI value (HBGI) is generated by generating a risk r(BG) as

r(BG)=10·(1.509·([log(BG)]^{1.084}−5.381))²

where BG refers to a collected measurement 1020. The risk r(BG) balances the amplitude of hypoglycemic and hyperglycemic ranges (enlarging the amplitude of hypoglycemic ranges and shrinking the amplitude of hyperglycemic ranges) and makes the transformed data symmetric around zero and fitting a normal distribution.

The HBGI value (HBGI) is generated as

$$HBGI = \frac{1}{{N}_{h}} {\sum }_{i = 1}^{{N}_{h}} rh ( {BG}_{i} )$$

where Nₕ refers to a number of glucose measurements greater than a threshold level (e.g., 112.5 mg/dL) and rh(BGᵢ) refers to the risk r(BG) values for each of the measurements greater than a threshold level (e.g., 112.5 mg/dL).

In one or more embodiments, the hypoglycemic risk value is based on a low blood glucose index (LBGI) value generated for SMBG readings. The LBGI value (LBGI) is generated as

$$LBGI = \frac{1}{{N}_{l}} {\sum }_{i = 1}^{{N}_{l}} rl ( {BG}_{i} )$$

where Nₗ refers to a number of glucose measurements less than a threshold level (e.g., 112.5 mg/dL) and rl(BGᵢ) refers to the risk r(BG) values for each of the measurements less than a threshold level (e.g., 112.5 mg/dL).

The content-based deviation detection module 1006 detects deviations from typical glucose level trends for the user in recent history by monitoring the aggregate metrics 1022. This is also referred to as content-based deviation detection because the detection is performed based on recent glucose measurements. The content-based deviation detection module 1006 receives the aggregate metrics 1022 and determines, based on the aggregate metrics 1022, whether the most recently collected measurements 1020 indicate a deviation in glucose level for user 102 relative to previously collected measurements 1020 (e.g., over the preceding 12 hours).

The content-based deviation detection module 1006 determines whether there is a deviation in glucose level for user 102 at regular or irregular intervals based on a preceding time period. In one or more implementations, the metric determination module 1004 generates aggregate metrics 1022 approximately every 30 minutes, and the content-based deviation detection module 1006 determines, in response to receiving new aggregate metrics 1022 from metric determination module 1004, whether there is a deviation in glucose level for user 102 approximately every 30 minutes by comparing the most recently received aggregate metrics 1022 to the 24 previously received aggregate metrics 1022 (e.g., the aggregate metrics 1022 received over the previous 12 hours). Aggregate metrics 1022 are stored, for example in an aggregate metrics store 1024, so the previously received aggregate metrics 1022 are readily available to the content-based deviation detection module 1006. The aggregate metrics store 1024 can be any of a variety of types of storage devices (e.g., random access memory, Flash memory, magnetic disk, and so forth).

Additionally or alternatively, other intervals or time periods can be used. For example, the metric determination module 1004 may generate one or more aggregate metrics 1022 approximately every 15 minutes, and the content-based deviation detection module 1006 determines, in response to receiving a new aggregate metric 1022 from metric determination module 1004, whether there is a deviation in glucose level for user 102 approximately every 15 minutes by comparing the most recently received aggregate metric 1022 to the aggregate metrics 1022 received over the previous 10 hours. By way of another example, the metric determination module 1004 may generate one or more aggregate metrics 1022 approximately every 5 minutes (using a rolling window of the 30 previous minutes in time), and the content-based deviation detection module 1006 determines, in response to receiving a new aggregate metric 1022 from metric determination module 1004, whether there is a deviation in glucose level for user 102 approximately every 5 minutes by comparing the most recently received aggregate metric 1022 to the aggregate metrics 1022 received over the previous 12 hours.

In one or more embodiments, the content-based deviation detection module 1006 receives the most recently generated aggregate metrics 1022 (e.g., the hypoglycemic risk value and hyperglycemic risk value most recently generated by the metric determination module 1004) and generates predicted aggregate metrics (e.g., a predicted hypoglycemic risk value and a predicted hyperglycemic risk value) based on the previously received aggregate metrics 1022. The content-based deviation detection module 1006 compares the predicted aggregate metric to the received aggregate metric and determines that there is a deviation in glucose level for user 102 relative to previously collected measurements 1020 if the predicted aggregate metric and the received aggregate metric differ by greater than a particular amount (e.g., greater than a threshold amount, such as 5.5). The content-based deviation detection module 1006 outputs indications of deviations detected based on the aggregate metrics 1022.

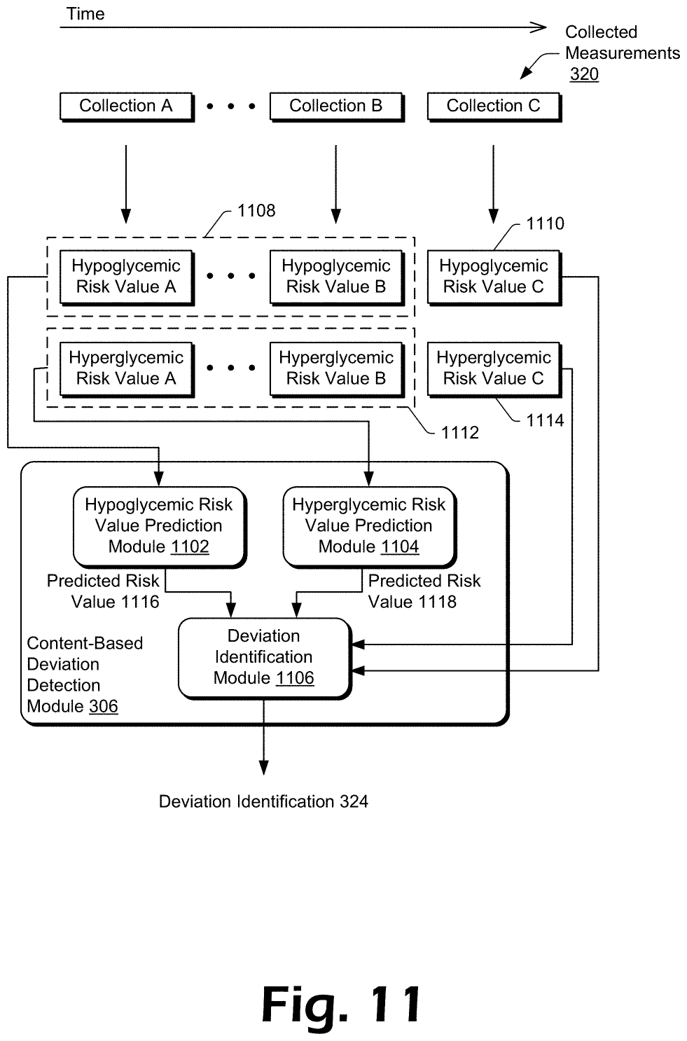

FIG. 11 is an illustration of an example implementation of the content-based deviation detection module 1006. In the example of FIG. 11 the aggregate metrics 1022 include a hypoglycemic risk value and a hyperglycemic risk value. Although discussed with reference to hypoglycemic and hyperglycemic risk values, it should be noted that the content-based deviation detection module 1006 can analogously use any other aggregate metrics in addition to, or in place of, the hypoglycemic and hyperglycemic risk values.

In the example of FIG. 11, the content-based deviation detection module 1006 includes a hypoglycemic risk value prediction module 1102, a hyperglycemic risk value prediction module 1104, and a deviation identification module 1106. Hypoglycemic risk values 1108 and 1110 and hyperglycemic risk values 1112 and 1114 are generated by the metric determination module 1004 from collected measurements 1020 as discussed above. As illustrated, a hypoglycemic risk value and a hyperglycemic risk value is generated for each collection of measurements 1020. Hypoglycemic risk values 1108 are provided to the hypoglycemic risk value prediction module 1102. The hypoglycemic risk value prediction module 1102 generates, based on the preceding hypoglycemic risk values 1108, a predicted risk value 1116 of the most recently received hypoglycemic risk value (hypoglycemic risk value 1110 in the example of FIG. 11). Similarly, hyperglycemic risk values 1112 are provided to the hyperglycemic risk value prediction module 1104. The hyperglycemic risk value prediction module 1104 generates, based on the preceding hyperglycemic risk values 1112, a predicted risk value 1118 of the most recently received hyperglycemic risk value (hyperglycemic risk value 1114 in the example of FIG. 11).

In one or more implementations, the hypoglycemic risk prediction module 1102 uses a machine learning system to generate the predicted risk value 1116 and the hyperglycemic risk value prediction module 1104 uses a machine learning system to generate the predicted risk value 1118. Machine learning systems refer to a computer representation that can be tuned (e.g., trained) based on inputs to approximate unknown functions. In particular, machine learning systems can include a system that utilizes algorithms to learn from, and make predictions on, known data by analyzing the known data to learn to generate outputs that reflect patterns and attributes of the known data. For instance, a machine learning system can include decision trees, support vector machines, linear regression, logistic regression, Bayesian networks, random forest learning, dimensionality reduction algorithms, boosting algorithms, artificial neural networks, deep learning, and so forth.

The machine learning system of hypoglycemic risk value prediction module 1102 is trained, for example, by using training data that is sets of multiple risk values. Each set of multiple (X) risk values includes a hypoglycemic risk value (values 1, . . . , X−1) for each of multiple collections of measurements. The machine learning system generates a predicted hypoglycemic risk value based on the training data value 1, . . . , X−1, and the machine learning system is trained by updating weights or values of layers in the machine learning system to minimize the loss between the predicted hypoglycemic risk value 1116 and the actual hypoglycemic risk value (value X). Various different loss functions can be used in training the machine learning system, such as cross entropy loss, mean squared error loss, and so forth.

Similarly, the machine learning system of hyperglycemic risk value prediction module 1104 is trained, for example, by using training data that is sets of multiple risk values. This training data can the same training data as is used to train the machine learning system of hypoglycemic risk value prediction module 1102. Each set of multiple (X) risk values includes a hyperglycemic risk value (values 1, . . . , X−1) for each of multiple collections of measurements. The machine learning system generates a predicted hyperglycemic risk value based on the training data value 1, . . . , X−1, and the machine learning system is trained by updating weights or values of layers in the machine learning system to minimize the loss between the predicted hyperglycemic risk value 1118 and the actual hyperglycemic risk value (value X). Various different loss functions can be used in training the machine learning system, such as cross entropy loss, mean squared error loss, and so forth.

In one or more embodiments, users are separated into different populations that have one or more similar characteristics. The user 102 is part of one of these different populations and the machine learning systems of the hypoglycemic risk value prediction module 1102 and the hyperglycemic risk value prediction module 1104 are trained using training data obtained from other users that are in the same population as the user 102 (e.g., and excluding any data obtained from users that are not in the same population as the user 102). The populations can be defined in any of a variety of different manners. In one or more embodiments, the populations are defined by diabetes diagnosis (e.g., the user does not have diabetes, the user has Type 1 diabetes, or the user has Type 2 non-insulin-dependent diabetes). Additionally or alternatively, the populations are defined in different manners, for example age-based populations. E.g., populations are based on whether the user is an adult or a child (e.g., older than 18 or younger than 18), based on an age bracket the user is in (e.g., 0-5 years old, 5-10 years old, 10-20 years old, 20-30 years old, etc.), and so forth. By way of another example, populations can be defined based on additional medical conditions a user may have, such as hypertension, obesity, cardiovascular disease, neuropathy, nephropathy, retinopathy, Alzheimer's, depression, and so forth. By way of another example, populations can be defined based on user habits or activities, such as exercise or other physical activities, sleep patterns, time spent working versus at leisure, and so forth. By way of another example, populations can be defined based on the manner in which glucose measurements 114 are obtained or the equipment used to obtain glucose measurements 114, such as whether glucose measurements 114 are obtained via CGM, a brand of wearable glucose monitoring device 104, a frequency with which glucose measurements 114 are obtained, and so forth.

By way of another example, populations can be defined based on past glucose measurements 114 for users, such as by grouping users by clustering based on past glucose measurements 114. Examples of such clusters include users with high glycemic variability, users with frequent hypoglycemia, users with frequent hyperglycemia, and so forth. By way of another example, users can be grouped by clustering by using the past activity data of the users (e.g., step counts, energy expenditure, exercise minutes, sleep hours, and so forth obtained from activity trackers worn by the users). Examples of such clusters include users with high average steps per day, users with low average energy expenditure per day, users with low average number of sleep hours, and so forth.

Separating users into different populations allows the glucose level deviation detection system 306 to be customized to the particular user 102, such as by training the machine learning system based on data from other users with similar characteristics. This improves the accuracy of the machine learning systems of the hypoglycemic risk value prediction module 1102 and the hyperglycemic risk value prediction module 1104 because data from users that differ from the particular user 102 need not be considered.

Although separate hypoglycemic risk value prediction module 1102 and hyperglycemic risk value prediction module 1104 are discussed, additionally or alternatively the content-based deviation detection module 1006 includes a single risk value prediction module that generates both the predicted risk value 1116 and the predicted risk value 1118 (and optionally additional aggregate metrics). E.g., a single machine learning system may be trained to generate both the predicted risk value 1116 and the predicted risk value 1118 (and optionally other predicted aggregate metrics). Additionally or alternatively, the content-based deviation detection module 1006 can include prediction modules to generate predicted aggregate metrics for other aggregate metrics (e.g., mean glucose, mean coefficient of variation, mean time in range, and so forth).

The deviation identification module 1106 determines, based on the predicted risk value 1116 and the actual risk value 1110 whether there is a hypoglycemic deviation in glucose level for the user 102. The deviation identification module 1106 can make this determination in any of a variety of different manners. In one or more embodiments, the deviation identification module 1106 determines that there is a deviation in glucose level for the user 102 in response to the predicted risk value 1116 and the actual risk value 1110 differing by at least a threshold amount. This threshold amount can be a fixed value (e.g., 5.5) or a variable value (e.g. 10% of the predicted risk value 1116 or of the actual risk value 1110).

Similarly, the deviation identification module 1106 determines, based on the predicted risk value 1118 and the actual risk value 1114 whether there is a hyperglycemic deviation in glucose level for the user 102. The deviation identification module 1106 can make this determination in any of a variety of different manners. In one or more embodiments, the deviation identification module 1106 determines that there is a deviation in glucose level for the user 102 in response to the predicted risk value 1118 and the actual risk value 1114 differing by at least a threshold amount. This threshold amount can be a fixed value (e.g., 5.5) or a variable value (e.g. 10% of the predicted risk value 1118 or of the actual risk value 1114).

The deviation identification module 1106 outputs a deviation indication 1026 indicating whether there is a deviation in glucose level (hyperglycemic or hypoglycemic) for user 102.

Thus, it can be seen that the deviation identification module 1106 focuses on the error in the predictions by hypoglycemic risk value prediction module 1102 and hyperglycemic risk value prediction module 1104. A large prediction error indicates that the glucose measurements in the collected measurements 1020 are changing in an unpredictable manner and thus are potentially deviating from the expected measurements.

Returning to FIG. 10, the contextual deviation detection module 1008 detects real-time deviations from typical repeating glucose level trends found in the extended history of glucose levels for the user. This is also referred to as context-based outlier detection because the detection is performed based on current glucose levels in the context of the extended history of glucose levels for the user (e.g., the preceding 3 days, the preceding 2 weeks, etc.). The contextual deviation detection module 1008 receives the aggregate metrics 1022 and determines, based on the aggregate metrics 1022, whether the most recently collected measurements 1020 (e.g., received over the preceding hour) indicate a deviation in glucose level for user 102 relative to previously collected measurements 1020 (e.g., over the corresponding time period in each of the preceding 3-14 days).

The contextual deviation detection module 1008 determines whether there is a deviation in glucose level for user 102 based on a recent time period and the corresponding time period in each of multiple preceding days. The contextual deviation detection module 1008 receives one or more aggregate metrics 1022 from the metric determination module 1004. Although illustrated as the same aggregate metrics, the content-based deviation detection module 1006 and the contextual deviation detection module 1008 optionally receive different aggregate metrics. For example, the content-based deviation detection module 1006 and contextual deviation detection module 1008 may receive aggregate metrics 1022 at different time intervals, based on collected measurements 1020 over different time periods or previous minutes of time, may receive aggregate metrics for different metrics (e.g., content-based deviation detection module 1006 may receive hypoglycemic and hyperglycemic risk values whereas contextual deviation detection module 1008 may receive mean glucose and mean amplitude of glycemic excursions metrics), and so forth.

In one or more implementations, the metric determination module 1004 generates aggregate metrics 1022 approximately every 5 minutes (using a rolling window of a particular time period, such as the 60 previous minutes in time) and the contextual deviation detection module 1008 determines, in response to receiving a new aggregate metric 1022 from metric determination module 1004, whether there is a deviation in glucose level for user 102 approximately every 5 minutes by comparing the received aggregate metric 1022 for the particular time period and the received aggregate metrics 1022 for the corresponding time period in each of multiple preceding days. The data collection module 1002 can generate a collection of measurements 1020 and the metric determination module 1004 can generate one or more aggregate metrics 1022 in response to receipt of a glucose measurement 114.

The contextual deviation detection module 1008 can compare the most recently generated aggregate metrics 1022 (e.g., the hypoglycemic risk value and hyperglycemic risk value most recently generated by the metric determination module 1004) to the aggregate metrics in the corresponding time period in each of multiple preceding days in any of a variety of different manners to determine whether there is a deviation in glucose level for the user 102.

In one or more embodiments, the contextual deviation detection module 1008 generates a value representing or combining the aggregate metrics for the corresponding time period in each of multiple preceding days. For example, this value can be any of a variety of statistics, such as the average of the risk values in each of the multiple preceding days (or in subgroups of the multiple preceding days), the mean of the risk values in each of the multiple preceding days (or in subgroups of the multiple preceding days), and so forth. The contextual deviation detection module 1008 determines that there is a deviation in glucose level for the user 102 in response to the difference between the value representing the aggregate metrics for the corresponding time period in each of multiple preceding days and the most recently generated aggregate metric 1022 being at least a threshold amount. This threshold amount can be a fixed value (e.g., 7) or a variable value (e.g. 10% of the most recently received aggregate metric 1022 or of the value representing the aggregate metrics for the corresponding time period in each of multiple preceding days).

The contextual deviation detection module 1008 can use any of a variety of rules or criteria to determine the number of preceding days (or which preceding days) to compare the most recently generated aggregate metrics to. For example, the contextual deviation detection module 1008 can compare the most recently generated aggregate metrics to the aggregate metrics for each preceding day over a pre-defined number of days, such as 14 days. The number of days can be pre-defined in various manners, such as by a developer or designer of the contextual deviation detection module 1008, by user input from the user 102, by input from a healthcare provider, and so forth. By way of another example, the contextual deviation detection module 1008 can compare the most recently generated aggregate metrics to the aggregate metrics for at least a particular number of days, then increasing the number (e.g., the aggregate metrics for the preceding 3 days, then the aggregate metrics for the preceding 4 days, then the aggregate metrics for the preceding 5 days, and so forth).

The contextual deviation detection module 1008 determines whether there is a deviation in glucose level for the user 102 based on the aggregate metrics generated by the metric determination module 1004. The contextual deviation detection module 1008 outputs a deviation indication 1028 indicating whether there is a deviation in glucose level (e.g., and an indication of the aggregate metric that resulted in the deviation in glucose level being identified) for user 102.

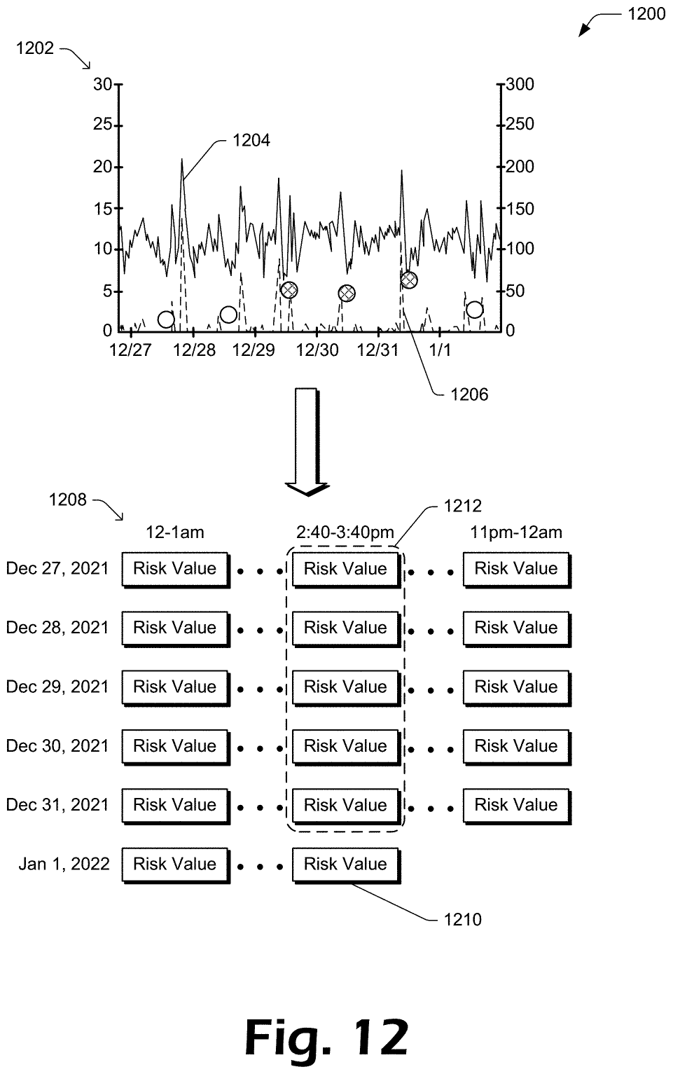

FIG. 12 illustrates an example 1200 of generating a deviation indication 1028. In the example of FIG. 12 the aggregate metrics 1022 include hypoglycemic risk values. Although discussed with reference to hypoglycemic risk values, it should be noted that the contextual deviation detection module 1008 can analogously use any other aggregate metrics in addition to, or in place of, the hypoglycemic risk values.

In the example of FIG. 12, the example 1200 shows a graph 1202 of glucose measurements and risk values over multiple days (December 27 through January 1). The graph 1202 shows glucose measurements on the right ranging from 0 to 300 and hyperglycemic risk values on the left ranging from 0 to 30. A solid line 1204 plots glucose measurements and a dashed line 1206 plots hyperglycemic risk value precursors. Multiple risk values 1208 are generated over 1-hour time periods.

In the illustrated example, the metric determination module 1004 generates a hyperglycemic risk value 1208 approximately every 5 minutes (using a rolling window of a 60-minute time period). The contextual deviation detection module 1008 determines, in response to receiving a new hyperglycemic risk value 1210 from metric determination module 1004, whether there is a deviation in glucose level for user 102 for the preceding 60-minute period on the current day and the hyperglycemic risk values 1212 for the corresponding time period in each of multiple preceding days. In the illustrated example, the contextual deviation detection module 1008 receives the risk value 1210 at approximately 3:40 pm on Jan. 1, 2022, and compares the risk value 1210 to the risk values 1212 corresponding to the same preceding 60 minutes (2:40-3:40 pm) from each of the days Dec. 27, 2021 through Dec. 31, 2021.

In the illustrated example, the contextual deviation detection module 1008 determines that there is a deviation in glucose level for the user 102 in response to the difference between the most recently generated hyperglycemic risk value and the value representing the hyperglycemic risk value for the three immediately preceding days being at least a threshold amount. The hyperglycemic risk values for each day are illustrated by circles in the graph 1202, with the hyperglycemic risk values for the three immediately preceding days being illustrated by circles with cross-hatched filling.

Returning to FIG. 10, the content-based deviation detection module 1006 and the contextual deviation detection module 1008 each allow glucose level deviations to be detected that the other module cannot detect. For example, glucose levels over a time period may be relatively consistent within a small range for the preceding 12 hours but be very different from the corresponding time periods in previous days. Thus, content-based deviation detection module 1006 would not detect a deviation but contextual deviation detection module 1008 would detect a deviation. By way of another example, glucose levels over a time period may be relatively consistent with the corresponding time periods in previous days but very different from the preceding 12 hours. Thus, contextual deviation detection module 1008 would not detect a deviation but content-based deviation detection module 1006 would detect a deviation.

The deviation selection module 1010 receives the deviation indication 1026 and the deviation indication 1028 from the content-based deviation detection module 1006 and the contextual deviation detection module 1008, respectively. The deviation selection module 1010 selects one or more of the deviation indication 1026 and the deviation indication 1028 and obtains a corresponding deviation identification 1030 from the deviation identification library 1032 (e.g., maintained on storage device 118). The obtained deviation identification 1030 is a message or communication that can be displayed or communicated to another device, user, healthcare professional, etc. that identifies or describes the corresponding deviation indication 1026 or deviation indication 1028. The deviation selection module 1010 provides the obtained corresponding deviation identification 1030 to UI module 1012 (or the feedback presentation system 122 as a feedback indication 312).

The deviation selection module 1010 determines which deviation identification 1030 corresponds to a deviation indication 1026 or a deviation indication 1028 in any of a variety of different manners. In one or more implementations, the deviation selection module 1010 maintains a mapping of deviation identifications 1030 to deviation indications. Additionally or alternatively, the deviation selection module 1010 may use any of a variety of other rules or criteria to determine which deviation identification 1030 corresponds to a deviation indication 1026 or a deviation indication 1028.

In one or more embodiments, the deviation selection module 1010 selects a deviation identification 1030 for each deviation identified in deviation indications 1026 and deviation indications 1028. This can result in deviation selection module 1010 selecting multiple deviation identifications 1030 that are displayed or otherwise presented by UI module 1012 (or the feedback presentation system 122).

Additionally or alternatively, in situations in which multiple deviation identifications are received, deviation selection module 1010 selects a subset (e.g., one) of the deviations. The deviation selection module 1010 can select one of the multiple deviations in various manners, such as randomly or pseudorandomly selecting one of the multiple deviations. Additionally or alternatively, the deviation selection module 1010 can prioritize the multiple deviations and select one of the multiple deviations having a highest priority. For example, the deviation having the highest priority is selected.

The deviation selection module 1010 optionally uses various criteria to determine which of the multiple deviations to select. These criteria can be based on various factors, such as how recently a deviation previously occurred, a ranking or prioritization of deviations or metrics, categories of deviations or metrics, how many consecutive days the deviations have occurred, and so forth. For example, a deviation that previously occurred less recently is selected over a deviation that occurred more recently. E.g., this allows different deviations to be selected and avoids repeatedly displaying the same deviation too frequently.

By way of another example, the deviation selection module 1010 may select deviations from one of content-based deviation detection module 1006 and contextual deviation detection module 1008 over the other. E.g., the deviation selection module 1010 may select a deviation identified by the content-based deviation detection module 1006 over a deviation identified by the contextual deviation detection module 1008. Or the deviation selection module 1010 may select a deviation from the one of content-based deviation detection module 1006 and contextual deviation detection module 1008 that least recently identified a deviation.

By way of another example, a deviation designated (e.g., by a developer or designer of the deviation selection module 1010) to be more urgent or safety-related is selected over a deviation that is less urgent or safety-related. E.g., this allows deviations corresponding to urgent or safety-related features (e.g., not staying within ranges or exceeding threshold glucose levels) to be selected over other non-urgent or non-safety-related features and display or otherwise present more critical diabetes management information to the user.

By way of another example, a deviation designated as being higher priority (e.g., by the user 102) may be selected over a deviation that is designated as being lower priority (e.g., by the user 102). E.g., this allows deviations that are of greater interest to the user to be displayed or otherwise presented rather than deviations that are of less interest to the user.

By way of another example, the deviation selection module 1010 may select a deviation only if it has not been selected for at least a threshold amount of time. E.g., the deviation selection module 1010 may select a deviation only if the deviation has not been selected for at least 30 minutes or for 2 days, the deviation selection module 1010 may select a particular deviation only if at least a threshold number (e.g., 3 or 5) other deviations have been selected since that particular deviation was last selected, and so forth.

By way of another example, the deviation selection module 1010 may select a deviation based on a population that the user 102 is a part of Populations can be defined or described in various manners as discussed above. As examples, certain deviations may be selected over other deviations based on whether the user is a Type 1 or Type 2 diabetic, based on how old the user is, based on other medical conditions the user has, and so forth.

By way of another example, the deviation selection module 1010 may select a deviation based on other factors or input from various medical sources. As examples, certain deviations may be selected over other deviations based on input from subject matter experts (e.g., experts in the field of diabetes management), clinical guidelines, professional literature, and so forth.

The UI module 1012 receives one or more deviation identifications 1030 and causes the deviation identifications 1030 to be displayed or otherwise presented (e.g., at computing device 106). This display or other presentation can take various forms, such as a static text display, graphic or video display, audio presentation, combinations thereof, and so forth. Additionally or alternatively, the one or more deviation identifications 1030 can be communicated to or otherwise presented to a clinician, pharmacist, other health care provider, and so forth.

The particular content or message presented by the UI module 1012 (or the feedback presentation system 122) for a deviation identification 1030 can vary. The content or message in the deviation identification 1030 can be any appropriate text or other content based on the selected deviation. Examples of such content or messages include “Your glucose is a little higher than usual,” “Your glucose is a little lower than usual,” “Your glucose is a bit higher than it has been in the afternoon over the last few days,” “Your glucose has risen quite a bit since this morning,” “Your glucose is increasing to the highest level it has been since this morning,” “Your glucose over the last hour is higher than it has been at this time for each of the past few days,” and so forth.

Accordingly, the content or message in the deviation identification 1030 can be a positive acknowledgement, such as a congratulatory acknowledgement of deviations trending away from normal behavior in a positive or self-serving way. Additionally or alternatively, the content or message in the deviation identification 1030 can be a preemptive warning, such as acknowledging negatively trending deviations to preempt worsening patterns.

The UI module 1012 can optionally communicate, display, or otherwise present deviation identification 1030 at any of a variety of different timings. In one or more embodiments, the UI module 1012 communicates, displays, or otherwise presents deviation identification 1030 in response to receiving the deviation identification 1030 from deviation selection module 1010 (e.g., at approximately the same time as the deviation is detected by content-based deviation detection module 1006 or contextual deviation detection module 1008). Additionally or alternatively, the UI module 1012 communicates, displays, or otherwise presents deviation identification 1030 at other times, such as at the completion of a meal, at regular or irregular intervals (e.g., approximately every 5 minutes, with the deviation selection module 1010 optionally selecting a different one of multiple deviation identifications 1030 every 5 minutes), in response to user input requesting a most recent deviation identification 1030, and so forth.

The glucose level deviation detection system 306 optionally takes additional actions based on detected deviations (or the lack thereof). In one or more implementations, these actions include notifying the glucose monitoring application 116 or the wearable glucose monitoring device 104 that the frequency with which glucose measurements 114 are produced can be reduced or increased. For example, if the glucose level deviation detection system 306 identifies a time period for which no deviation is detected (e.g., for multiple days), the glucose level deviation detection system 306 notifies the glucose monitoring application 116 or wearable glucose monitoring device 104 that the frequency with which glucose measurements 114 are produced can be reduced (e.g., from every 5 minutes to every 10 minutes) during that time period, reducing the power expended to produce glucose measurements 114. By way of another example, if the glucose level deviation detection system 306 identifies a deviation for a time period, the glucose level deviation detection system 306 notifies the glucose monitoring application 116 or wearable glucose monitoring device 104 that the frequency with which glucose measurements 114 are produced can be increased (e.g., from every 5 minutes to every 2 minutes) during that time period on subsequent days or during subsequent time periods on the current day, increasing the accuracy of the generated risk values due to the increased number of glucose measurements 114.

Although discussed as using the aggregate metrics 1022, additionally or alternatively the collected measurements 1020 are provided to one or both of the content-based deviation detection module 1006 and the contextual deviation detection module 1008. In such situations, the content-based deviation detection module 1006 or the contextual deviation detection module 1008 identify deviations analogous to the discussion above but using the collected measurements 1020 rather than the aggregate metrics 1022.

Furthermore, glucose level deviation detection system 306 is discussed as including both content-based deviation detection module 1006 and contextual deviation detection module 1008, which can operate concurrently to generate deviation indications. Additionally or alternatively, the glucose level deviation detection system 306 includes only one of the content-based deviation detection module 1006 and the contextual deviation detection module 1008.

It should be noted that, as discussed above, the deviation identification 1030 can be provided to the feedback presentation system 122 as feedback indications 312. In such situations the glucose level deviation detection system 306 need not include the UI module 1012. Additionally or alternatively, the deviation indications 1026 and 1028 can be provided to the feedback presentation system 122 as feedback indications 312. In such situations the feedback presentation system 122 identifies deviations to be provided to the user (or others, such as a clinician or pharmacist), optionally using the feedback library 1032 as discussed in more detail below. The feedback presentation system 122 optionally identifies deviations to be identified to the user using any one or more of the techniques discussed herein with respect to the deviation selection module 1010.

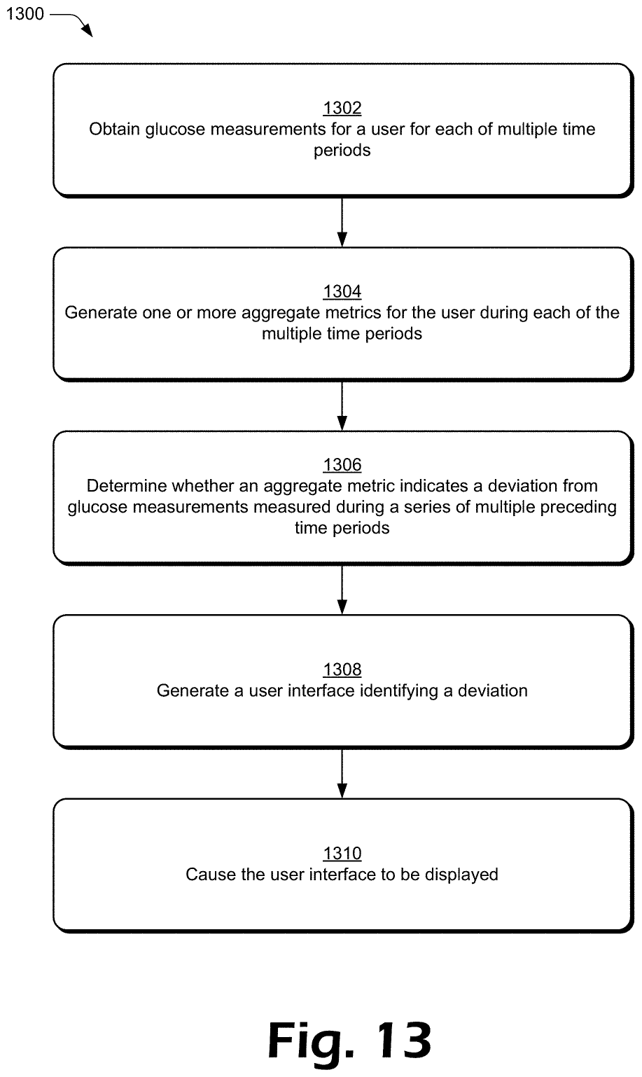

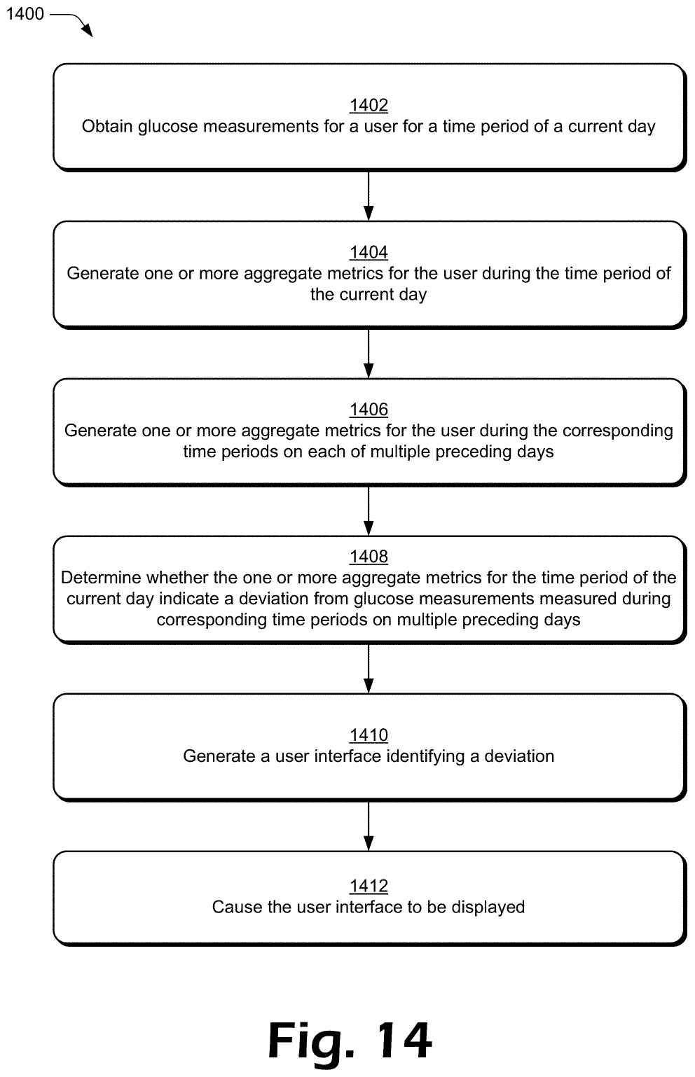

FIG. 13 and FIG. 14 describe examples of procedures for implementing glucose level deviation detection. Aspects of the procedures may be implemented in hardware, firmware, or software, or a combination thereof The procedures are shown as a set of blocks that specify operations performed by one or more devices and are not necessarily limited to the orders shown for performing the operations by the respective blocks.

FIG. 13 depicts a procedure 1300 in an example of implementing glucose level deviation detection. Procedure 1300 is performed, for example, by a glucose level deviation detection system, such as the glucose level deviation detection system 306 and optionally in part by a feedback presentation system, such as the feedback presentation system 122.

Glucose measurements for a user for each of multiple time periods are obtained (block 1302). These glucose measurements are obtained from a glucose sensor of, for example, a continuous glucose level monitoring system with the glucose sensor being inserted at an insertion site of the user. These time periods are, for example, 30-minute periods of time.

One or more aggregate metrics are generated for the user during each of the multiple time periods (block 1304). These one or more aggregate metrics can include, for example, hyperglycemic and hypoglycemic risk values (e.g., high blood glucose index and low blood glucose index), mean glucose, mean coefficient of variation, mean time in range, and so forth.

An aggregate metric indicates a deviation from glucose measurements measured during a series of multiple preceding time periods (block 1306). For example, the determination is made as to whether the most recently generated aggregate metric indicates a deviation from glucose measurements measured during the preceding 12 hours.

A user interface identifying the deviation is generated (block 1308). In some situations multiple deviations may be indicated in block 1304, and in such situations one or more of the identified deviations is selected for inclusion in the user interface in block 1308.

The user interface including the identified deviation is caused to be displayed (block 1310) or otherwise presented. Additionally or alternatively, the identified or selected deviation can be communicated to or otherwise presented to a clinician, pharmacist, or other health care provider.

FIG. 14 depicts a procedure 1400 in another example of implementing glucose level deviation detection. Procedure 1400 is performed, for example, by a glucose level deviation detection system, such as the glucose level deviation detection system 306 and optionally in part by a feedback presentation system, such as the feedback presentation system 122.

Glucose measurements for a user for a time period of a current day are obtained (block 1402). These glucose measurements are obtained from a glucose sensor of, for example, a continuous glucose level monitoring system with the glucose sensor being inserted at an insertion site of the user. This time period is, for example, 30-minute periods of time.

One or more aggregate metrics are generated for the user during the time period of the current day (block 1404). These one or more aggregate metrics can include, for example, hyperglycemic and hypoglycemic risk values (e.g., high blood glucose index and low blood glucose index), mean glucose, mean coefficient of variation, mean time in range, and so forth.

An aggregate metric is generated for the user during the corresponding time period on each of multiple preceding days (block 1406). For example, these corresponding time periods are the same 30-minute periods of time as the time period of the current day. This aggregate metric is the same aggregate metric as at least one of the aggregate metrics generated in block 1404. A determination is made as to whether the aggregate metric for the time period of the current day indicates a deviation from glucose measurements measured during corresponding time periods on the multiple preceding days (block 1408).

A user interface identifying the deviation is generated (block 1410). In some situations multiple deviations may be indicated in block 1408, and in such situations one or more of the identified deviations is selected for inclusion in the user interface in block 1410.

The user interface including the identified deviation is caused to be displayed (block 1412) or otherwise presented. Additionally or alternatively, the identified or selected deviation can be communicated to or otherwise presented to a clinician, pharmacist, or other health care provider.

### Behavior Modification Identification System Architecture

Generally, a behavior modification identification system 308 receives a data stream of glucose measurements. One or more features for a particular time period are generated and stored, each feature being a value that can be computed from the glucose measurements and that indicates whether the user has been engaging in beneficial diabetes management behaviors. The features may include metrics that are a representation or summarization of the data in the data stream for a particular time period. These time periods are, for example, different multi-hour blocks of time during a day. E.g., a day may include a first time period from midnight to 6 am (corresponding to sleep or night), a second time period from 6 am to noon (corresponding to after breakfast), a third time period from noon to 6 pm (corresponding to after lunch), and a fourth time period from 6 pm to midnight (corresponding to after dinner). These time periods may be fixed or may be adaptively identified based on various received data (e.g., sleep onset may be detected by an activity monitor and may be used to determine the beginning of the “sleep” time period on that date, user input may specify beginning or ending times for a time period (e.g., user input received via a user preferences user interface displayed to the user), and so forth).

The features for a time period are aggregated over a time window, such as one week. These aggregated features are used to identify patterns indicating that beneficial diabetes management behaviors are not being engaged in. For example, one feature may be a time in range feature (e.g., the range being glucose levels between 70 milligrams per deciliter (mg/dL) and 180 mg/dL) and a pattern indicating that beneficial diabetes management behaviors are not being engaged in may be that the time in range for a particular time period over a week is less than 70%. Potential behavior modification feedback is generated, for each of the identified patterns, that a user could take to engage in beneficial diabetes management behavior. At least one of the potential behavior modification feedback is selected and displayed or otherwise presented to the user.

Behavior modification feedback, also referred to as an actionable goal, refers to one or more actions that the user can take to alter (e.g., improve) his or her diabetes management. Examples of behavior modifications include “Take an evening walk 3 times this week,” “Eat a dinner low in carbohydrates 2 nights this week,” “To not eat close to bedtime, try setting a time that you will stop eating after each evening,” and so forth.

The techniques discussed herein generate behavior modification feedback for improving diabetes management and provide notifications of such to the user. This provides goals or behavior modification feedback to the user in a way that is informative and actionable for the user to improve their health, longevity, diabetes management, and so forth. This can allow the user to make appropriate changes in their lifestyle, reducing the need to monitor their glucose levels closely if they follow the behavior modification feedback.

Furthermore, the techniques discussed herein allow goals or suggestions of how to improve diabetes management to be generated and presented to the user. Thus, rather than simply displaying raw glucose data, the techniques discussed herein allow useful actions or steps to take to be identified to the user so that they can improve their diabetes management without having to try to figure out how to do so based on the raw glucose data alone.

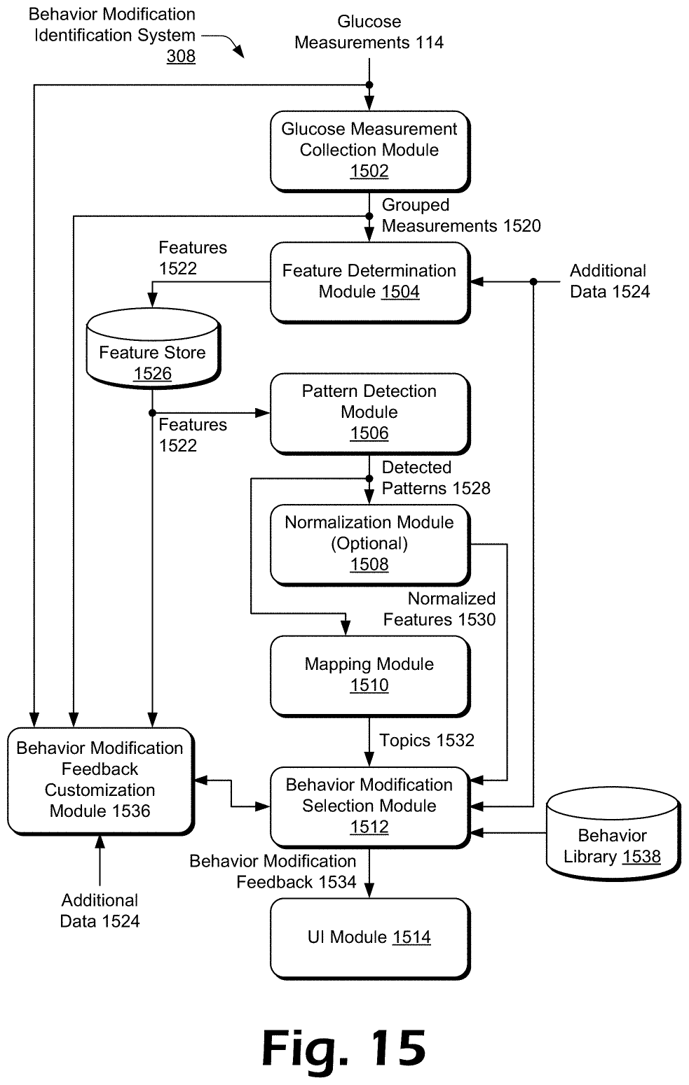

FIG. 15 is an illustration of an example architecture of a behavior modification identification system 308. The behavior modification identification system 308 includes a glucose measurement collection module 1502, a feature determination module 1504, a pattern detection module 1506, a normalization module 1508, a mapping module 1510, a behavior modification selection module 1512, and a UI module 1514 (optional). Generally, the behavior modification identification system 308 analyzes the glucose measurements 114 for the user 102 and looks for patterns in the glucose measurements 114 that indicate poor (or non-optimal) diabetes management by the user. Poor diabetes management can be quantified in various manners, such as glucose measurements 114 not staying within a particular range, glucose measurements 114 varying by greater than a particular amount, and so forth. In one or more implementations, the behavior modification identification system 308 identifies poor diabetes management by identifying patterns in glucose measurements 114 for a given time period of a time window across multiple time windows (e.g., for a given multi-hour time period, such as 6 am to noon, on each of multiple days).

The glucose measurement collection module 1502 receives glucose measurements 114 and optionally timestamps indicating when each of the glucose measurements 114 was taken (e.g., by wearable glucose monitoring device 104) or received (e.g., by glucose monitoring application 116). The timestamp may be provided, for example, by the wearable glucose monitoring device 104 or the glucose monitoring application 116. The glucose measurement collection module 1502 groups the glucose measurements 114 into different time periods referred to as grouped measurements 1520.

In one or more implementations, each time period is a portion of a day (or other 24 hour interval). These time periods are chosen to capture the impacts of specific diabetes management decisions and lifestyle choices. In one or more implementations, each day is separated into multiple time periods based on when the user eats meals and when the user sleeps. For example, a day may include a first time period from midnight to 6 am (corresponding to sleep or night), a second time period from 6 am to noon (corresponding to after breakfast), a third time period from noon to 6 pm (corresponding to after lunch), and a fourth time period from 6 pm to midnight (corresponding to after dinner). Additionally or alternatively, additional time periods can correspond to other user actions that affect glucose levels, such as when the user exercises.

The glucose monitoring application 116 optionally provides a user interface via which the user 102 can customize the time periods to his or her typical schedule. For example, assume the user 102 typically goes to bed at 10 pm, eats breakfast at 7 am, eats lunch at noon, and eats dinner at 5 pm. These times can be provided to the glucose monitoring application 116 (e.g., by the user), which determines the time periods for the day to include a first time period from 10 pm to 7 am (corresponding to sleep or night), a second time period from 7 am to noon (corresponding to after breakfast), a third time period from noon to 5 pm (corresponding to after lunch), and a fourth time period from 5 pm to midnight (corresponding to after dinner). A day may be separated into other numbers of periods than four. For example, assume the user 102 typically goes to bed at 10 pm, exercises at 5 am, eats breakfast at 7 am, eats lunch at llam, eats an afternoon snack at 2 pm, and eats dinner at 6 pm. These times can be provided to the glucose monitoring application 116, which determines the time periods for the day to include a first time period from 10 pm to 5 am (corresponding to sleep or night), a second time period from 5 am to 7 am (corresponding to exercise), a third time period 7 am to 11 am (corresponding to after breakfast), a fourth time period from 11 am to 2 pm (corresponding to after lunch), a fourth time period from 2 pm to 6 pm (corresponding to snack), and a sixth time period from 6 pm to 10 pm (corresponding to after dinner).

Additionally or alternatively, different time periods for the user 102 can be automatically learned by the glucose monitoring application 116 by monitoring various data available to the glucose monitoring application 116 (e.g., exercise or sleep patterns from an activity tracker, eating patterns from a food or calorie tracking application) or detected directly (e.g., sleep onset detected by activity tracker). Various rules or criteria can be used to determine time periods based on the various data available to the glucose monitoring application 116, such as detecting sleep onset and sleep cessation from an activity tracker and using the times of sleep onset and sleep cessation to determine the time period corresponding to sleep.

In one or more implementations, the glucose monitoring application 116 uses a machine learning system to determine the different time periods for the user 102. Machine learning systems refer to a computer representation that can be tuned (e.g., trained) based on inputs to approximate unknown functions. In particular, machine learning systems can include a system that utilizes algorithms to learn from, and make predictions on, known data by analyzing the known data to learn to generate outputs that reflect patterns and attributes of the known data. For instance, a machine learning system can include decision trees, support vector machines, linear regression, logistic regression, Bayesian networks, random forest learning, dimensionality reduction algorithms, boosting algorithms, artificial neural networks, deep learning, and so forth.

The machine learning system is trained, for example, by using training data that is sets of multiple data (e.g., times of exercise, sleep, or eating during a day) and timestamps indicating when the exercise, sleep, or eating was done. Known labels are associated with the sets of multiple data indicating a time period that the data corresponds to. The machine learning system is trained by updating weights or values of layers in the machine learning system to minimize the loss between time periods generated by the machine learning system for the training data and the corresponding known labels for the training data. Various different loss functions can be used in training the machine learning system, such as cross entropy loss, mean squared error loss, and so forth.

In one or more implementations the machine learning system is trained over time as the glucose monitoring application 116 is used over time. E.g., the user can provide an indication of whether a particular time period is correct, and this indication can be used as a known label for the current time periods and used to further train the machine learning system.

Accordingly, different time periods can be established for different users. Furthermore, different time periods can be established for different days. For example, the user 102 may have different schedules on different types of days (e.g., a different schedule on weekends and holidays than on weekdays, a different schedule on days the user works than on days the user does not work). Accordingly, the time periods for different types of days can be provided by the user 102 or determined by a machine learning system of the glucose monitoring application 116.

In one or more embodiments, the blocks of times for different time periods can vary for a user across different days. For example, a user may typically go to sleep between 11 pm and midnight, and wake up between 5:30 am and 6:30 am. For any given day, the time the user goes to sleep and the time the user wakes up can be detected using various data streams, such as data from an activity tracker worn by the user. Accordingly, the time period corresponding to sleep for the user may be 11:13 pm to 6:00 am for one day, 11:27 pm to 5:48 am the next day, 11:45 pm to 6:12 am the next day, and so forth.

The feature determination module 1504 generates one or more features 1522 based on the grouped measurements 1520. A feature 1522 refers to any value that can be computed from the glucose measurements 114 (and optionally additional data) and that indicates whether the user has been engaging in beneficial diabetes management behaviors or lifestyle choices. A feature 1522 can be a metric that is a representation or summarization of the data in the glucose measurements 114 or for a particular time period during the time window.

In one or more implementations, the feature determination module 1504 also receives additional data 1524 (e.g., the additional data 302 of FIG. 3). The additional data 1524 refers to any additional data that may be used to identify poor diabetes management. For example, the additional data 1524 can include data that relates to user interactions with the computing device 106, with the display of the computing device 106, or with other system components that indicate level of engagement with diabetes management. E.g., this data can include timestamps of when the user 102 viewed the application as well as what screens or portions of the UI were viewed, timestamps of when the user 102 provided input to (or otherwise interacted with) the application 116 as well as what that input was, timestamps of when the user viewed or acknowledged feedback provided by behavior modification identification system 308, the number of times an application (e.g., glucose monitoring application 116) is viewed or launched, the timing of when an application (e.g., glucose monitoring application 116) is viewed or launched, the time spent reviewing glucose data or previous insights or educational materials, the frequency of interactions with coaches or clinicians, and so forth. Such data can be received from various sources, such as from the glucose monitoring application 116, from an operating system running on the computing device 106, from the glucose monitoring platform 110, and so forth. The additional data 1524 may also include other data from other sources as discussed in more detail below.

In one or more implementations, each feature 1522 is one or two values that represent or summarize the glucose measurements 114 or additional data 1524 for a particular time period across the time window, transforming the glucose measurements 114 into a numeric indicator of the adherence to beneficial diabetes management and lifestyle choices. For example, each feature 1522 can be a value that represents or summarizes the glucose measurements 114 across a week for the time periods corresponding to sleep during the week.

The feature determination module 1504 generates, for corresponding time periods in a time window, any of a variety of features 1522. In one or more implementations, the feature determination module 1504 generates any of a variety of statistics from the glucose measurements 114, such as mean glucose measurement in the corresponding time periods, coefficient of variation for the glucose measurements in the corresponding time periods (the ratio of the standard deviation to the mean for the glucose measurements in the time periods), standard deviation of the glucose measurements in the time periods, receiver operating characteristics (ROC) and so forth.

Additionally or alternatively, the feature determination module 1504 generates a time in range feature, such as an amount of time during the time periods the glucose measurements were in an acceptable or desired range of glucose levels, e.g., between 70 mg/dL and 180 mg/dL, or a narrow range between 70 mg/dL and 130 mg/dL. This acceptable or desired range can be a default range, can be a custom range set by the user or a health care professional, and so forth. Different time in range features having different acceptable or desired ranges of glucose levels can be generated for different corresponding time periods (e.g., the range of glucose levels for the time periods corresponding to sleep may be different than the range of glucose levels for the time periods corresponding to after lunch).

Additionally or alternatively, the feature determination module 1504 generates a time above threshold feature, such as an amount of time during the time periods the glucose measurements were above a particular glucose level (e.g., 180 mg/dL or 250 mg/dL). This particular glucose level can be a default level, can be a custom level set by the user or a health care professional, and so forth. Multiple time above threshold features each having a different particular glucose level can be generated.

Additionally or alternatively, the feature determination module 1504 generates a time below threshold feature, such as an amount of time during the time periods the glucose measurements were below a particular glucose level (e.g., 70 mg/dL). This particular glucose level can be a default level, can be a custom level set by the user or a health care professional, and so forth. Multiple time below threshold features each having a different particular glucose level can be generated.

Additionally or alternatively, the feature determination module 1504 generates a maximum glucose measurement feature that is the maximum glucose measurement received during the time periods.

Additionally or alternatively, the feature determination module 1504 generates a post-prandial feature, such as post-prandial glucose level peak, post-prandial area under the curve (AUC), an amount of post-prandial time the glucose measurements were above a particular glucose level (e.g., 250 mg/dl), and so forth.

Additionally or alternatively, the feature determination module 1504 generates a fasting glucose in range feature, such as an indication (e.g., true or false) indicating whether a particular glucose measurement was in an acceptable or desired range of glucose levels, e.g., between 70 milligrams per deciliter (mg/dL) and 180 mg/dL, or a narrow range between 70 mg/dL and 130 mg/dL. This acceptable or desired range can be a default range, can be a custom range set by the user or a health care professional, and so forth. Different time in range features having different acceptable or desired ranges of glucose levels can be generated for different corresponding time periods. For example, a fasting glucose in range feature can be generated based on a glucose measurement received just prior to the first food the user eats each morning, one of the last glucose measurements received at the end of the time periods corresponding to sleep, and so forth. By way of another example, a bedtime glucose in range feature can be generated based on a glucose measurement received at the beginning of the time period corresponding to sleep, and so forth.

Additionally or alternatively, the feature determination module 1504 generates other features, such as maximum glucose measurement rate of change in the time periods, maximum glucose measurement rise in the time periods, low blood glucose index (LBGI) in the time periods, high blood glucose index (HBGI) in the time periods, a value indicating a rate of increase or decrease in glucose levels in the time periods, and so forth.

In one or more implementations, the feature determination module 1504 generates features from additional data 1524, which can be various different types of data received from various different sources as discussed herein. For example, the feature determination module 1504 can generate as features 1522 a number of times the glucose monitoring application 116 was viewed or launched in the time periods, the number of times the glucose monitoring application 116 was launched or viewed after meals (e.g., at the beginning of time periods corresponding to after breakfast, after lunch, after dinner, etc.), and so forth.

The feature determination module 1504 stores the generated features 1522 in a feature store 1526 (e.g., maintained on storage device 118). The generated features 1522 are maintained for a duration time that can vary by implementation. For example, the generated features 1522 may be maintained for two weeks, one month, one year, and so forth.

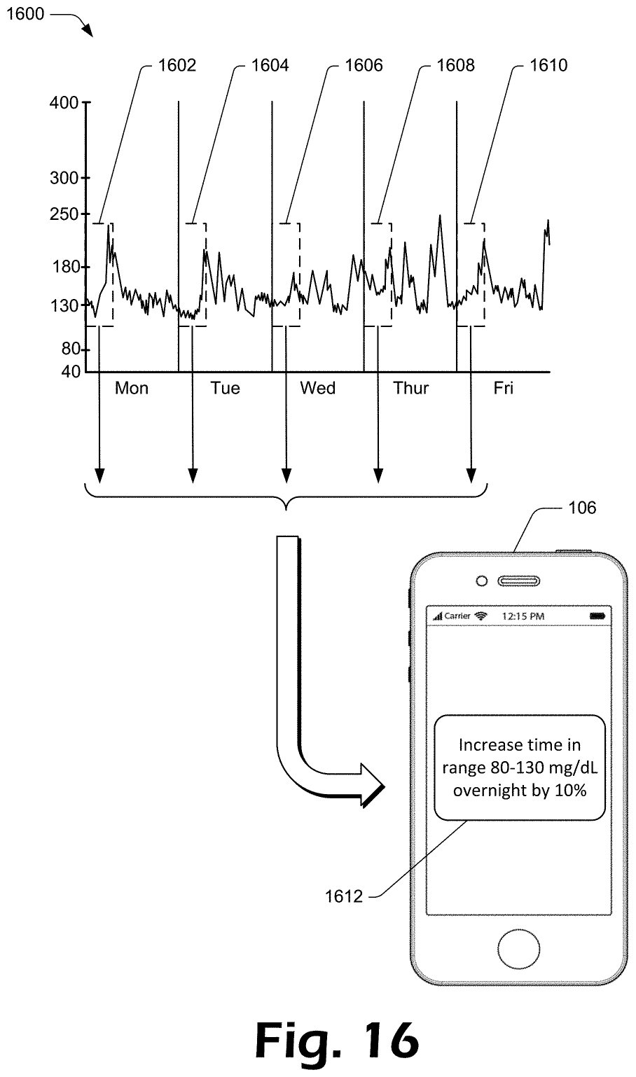

FIG. 16 illustrates an example 1600 of providing behavior modification recommendations for improving diabetes management. The example 1600 shows a time window of multiple days (illustrated as Monday, Tuesday, Wednesday, Thursday, and Friday) along the horizontal axis and glucose measurements along the vertical axis. Each day has multiple time periods (e.g., night, breakfast, lunch, and dinner) and the glucose measurements during the night time periods in each of the days are illustrated as 1602, 1604, 1606, 1608, and 1610. A time in range feature 1522 is generated for the corresponding time periods (e.g., the night time periods) with a range of 80-130 mg/dL. In the illustrated example 1600, the time in range feature 1522 is approximately 0.37 (37% of the night time periods are in range). As discussed in more detail below, a pattern is detected given the time in range feature 1522 for the night time periods, resulting in behavior modification feedback 1612 being displayed on the computing device 106.

Returning to FIG. 15, the pattern detection module 1506 receives the different features 1522 (e.g., from feature store 1526 or directly from feature determination module 1504) and detects, from the features 1522, patterns in corresponding time periods of a time window. These patterns are patterns that indicate poor (or non-optimal) diabetes management by the user. The pattern detection module 1506 can use any of a variety of rules, criteria, or other techniques to identify these patterns.

The pattern detection module 1506 identifies patterns based on the features 1522 from corresponding time periods in the time window (e.g., patterns in the night time period, patterns in the breakfast time period, patterns in the lunch time period, patterns in the dinner time period, and so forth). The pattern detection module 1506 can identify the same or different patterns in the different corresponding time periods. E.g., a pattern may be detected for the night time period and the lunch time period given the time in range feature 1522 for those time periods, but no such pattern may be detected for the breakfast and dinner time periods.

In one or more implementations, the pattern detection module 1506 uses rules based on target criteria for features 1522 that indicate desired values for the features 1522. Table I illustrates examples of features 1522 and their corresponding target criteria.

TABLE I

| Feature | Criteria |
|---|---|
| Mean | The mean for the glucose measurements in the |
| | corresponding time periods is less than 155 mg/dL |
| Time in | The glucose measurements in the corresponding time |
| range | periods (other than night sleep time periods) are in |
| (not night) | the range of 70-180 mg/dL greater than 70% of the time |
| Time in | The glucose measurements in the night or sleep time |
| range | periods are in the range of 80-130 mg/dL greater than |
| (night) | 70% of the time |
| Time above | The glucose measurements in the corresponding time |
| 180 | periods are above 180 mg/dL less than 25% of the time |
| Time above | The glucose measurements in the corresponding time |
| 250 | periods are above 250 mg/dL less than 5% of the time |
| Time below | The glucose measurements in the corresponding time |
| 70 | periods are below 70 mg/dL less than 1% of the time |
| Max | The maximum glucose measurement in the corresponding |
| glucose | time periods is less than 180 |
| Coefficient | The coefficient of variation for the glucose measurements |
| of variation | in the corresponding time periods is less than 36% |
| Fasting | The fasting glucose is in the range of 80-130 mg/dL |
| glucose | |
| Bedtime | The bedtime glucose is in the range of 80-180 mg/dL |
| glucose | |

The pattern detection module 1506 detects, as a pattern that indicates poor (or non-optimal) diabetes management by the user, each feature that does not satisfy its criteria. For example, if the mean for glucose measurements in the corresponding time periods (e.g., the time periods corresponding to after breakfast) is not less than 155 mg/dL, then the pattern detection module 1506 detects the glucose measurements for the mean feature in the after breakfast time period as a pattern that indicates poor diabetes management. By way of another example, if the glucose measurements in the corresponding time periods (e.g., the time periods corresponding to after lunch) are in the range of 70-180 mg/dL greater than 70% of the time, then the pattern detection module 1506 does not detect the time in range (not night) feature in the after lunch time period as a pattern that indicates poor diabetes management.

The pattern detection module 1506 outputs the detected patterns (the features 1522 that did not satisfy their criteria) during the time window (e.g., all of the detected patterns for the various features 1522 in the various corresponding time periods in the time window) as detected patterns 1528. Each detected pattern 1528 includes an indication of the detected pattern (e.g., the feature for which the pattern was detected and the corresponding time periods in which the pattern was detected). In one or more implementations, each detected pattern 1528 also includes an indication of the feature for which the pattern was detected. For example, if a pattern was detected for the time periods corresponding to after lunch not being in the range of 70-180 mg/dL greater than 70% of the time, the detected pattern 1528 includes the amount of time that the glucose measurements were in the range of 70-180 mg/dL for the time periods corresponding to after lunch (e.g., 45%).

In one or more implementations, the detected patterns 1528 (or at least the features for which the patterns were detected) are provided to a normalization module 1508, which adjusts the features for the detected patterns 1528 to a common scale or common units (e.g., a value ranging between 0 and 100, or between 0 and 1). The normalization module 1508 outputs the normalized features as normalized features 1530. This normalization can be performed using any of a variety of public or proprietary techniques. It should be noted that the normalization module 1508 is optional and that normalization need not be performed in certain situations. For example, in some situations if only features having a common scale or common units are used by the pattern detection module 1506 (e.g., the time above 180 and the time above 250 features) then there is no need to adjust the features for the detected patterns 1528 to a common scale or units.

In one or more embodiments, the normalization performed by normalization module 1508 indicates a size of the pattern, and an indication of this size is included in the normalized features 1530. The size of the pattern indicates how poorly the criteria for the feature was satisfied. For example, if for a time in range feature, if the time in the particular range (e.g., 70-180 mg/dL) is 45% but the criteria is to be at least 70%, then the size of this

$$100 * ( 1 - \frac{45}{70} )$$

pattern can be calculated as for a size of 35.7, whereas if the time in a different range (e.g., 80-10 mg/dL) is 68% but the criteria is to be at least 70%, then the

$$100 * ( 1 - \frac{68}{70} )$$

size of this pattern can be calculated as for a size of 2. These sizes allow the behavior modification selection module 1512 to select behavior modifications based on which pattern has the largest size (e.g., is considered worse or corresponds to the poorer diabetes management behavior).

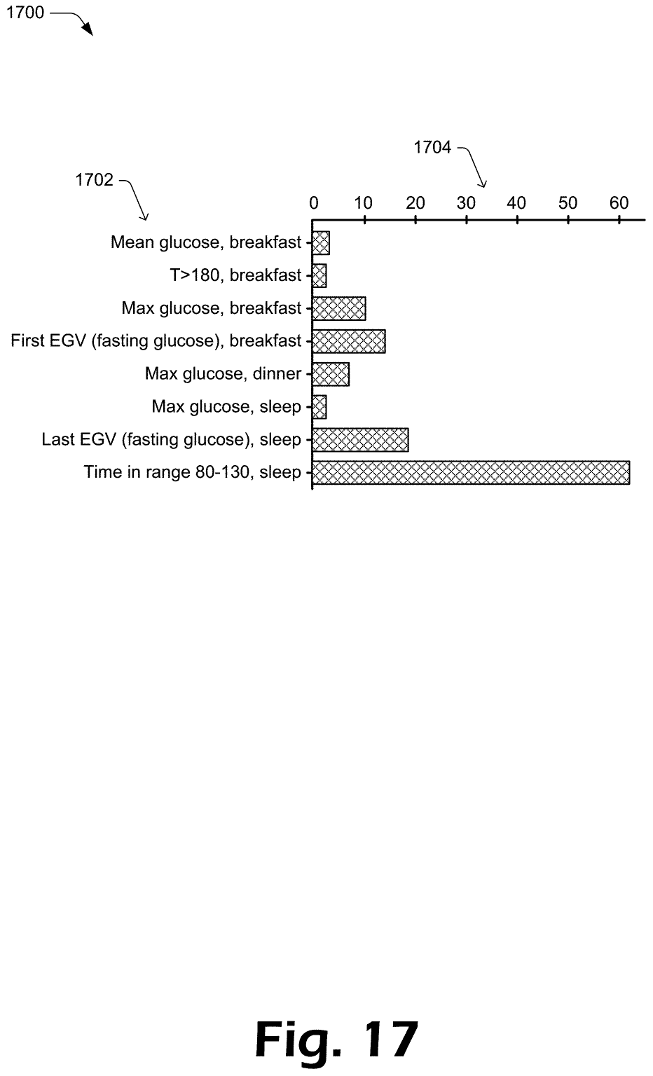

FIG. 17 illustrates an example 1700 of sizes of normalized sizes for different detected patterns. The detected patterns (and the time period in which they are detected) 1702 are illustrated along the vertical axis, and the sizes 1704 are illustrated along the horizontal axis. As illustrated, the detected pattern for the time in range 80-130 mg/dL during the sleep time period has the largest size, which may lead to the behavior modification selection module 1512 selecting behavior modification feedback that the time in range 80-130 mg/dL during the sleep time period maps to.

Returning to FIG. 15, in one or more implementations the various patterns that can be detected by the pattern detection module 1506 correspond to (are mapped to) one or more topics. The mapping module 1510 receives the detected patterns 1528 (and optionally the normalized features 1530) and maps the detected patterns 1528 to one or more topics 1532. The topics 1532 are also referred to as mapping to one or more patterns. Various behavior modification feedback are grouped into multiple different topics, also referred to as categories. Each such topic includes one or more patterns that are mapped to one or more behavior modification feedback. The mapping module 1510 maps the detected patterns 1528 to one or more topics 1532, and the behavior modification selection module 1512 selects behavior modification feedback (from a behavior library 1538, which can be maintained on storage device 118) corresponding to those one or more topics 1532 to provide to the UI module 1514 (or the feedback presentation system 122) for output as discussed in more detail below. In one or more implementations, the behavior library 1538 is the feedback library 124 of FIG. 1. Which detected patterns map to which topic or topics can be specified in various manners, such as by a developer or designer of the behavior modification identification system 308, by a health care provider or professional, and so forth.

The mapping module 1510 can map the detected patterns 1528 to any of a variety of different topics. For example, one topic of behavior modifications is engagement with a glucose monitoring application, such as glucose monitoring application 116. Patterns that can be mapped to this topic include low engagement with the glucose monitoring application as measured by, e.g., a low number (e.g., less than a threshold number, such as a fixed number (e.g., 3) or a variable number (e.g., less than 2 per hour)) of screen views or launches of the application, no screen views before or after meals, and so forth. In one or more implementations, patterns detected in any of the time periods can be mapped to the engagement with a glucose monitoring application topic. The engagement with a glucose monitoring application topic can be mapped to behavior modification feedback of: 1) check your glucose X number of times per day, 2) check your glucose every day at specified times (e.g., before/after meals, at bedtime, in the morning), 3) set an alarm to remind you to check your glucose, and so forth.

By way of another example, one topic of behavior modifications is post-prandial glucose. Patterns that can be mapped to this topic include high post-prandial glucose peak (e.g., greater than a threshold value, such as a fixed value (e.g., 300 mg/dL) or a variable number (e.g., the highest value the user has had during the time period over a duration of time, such as 2 weeks)), high post-prandial area under the curve (AUC) (e.g., greater than a threshold value, such as a fixed value (e.g., 300 mg/dL) or a variable number (e.g., the highest value the user has had during the time period over a duration of time, such as 2 weeks)), high post-prandial time with glucose levels greater than 250 mg/dl (e.g., greater than a threshold amount of time, such as a fixed amount of time (e.g., 30 minutes) or a variable amount of time (e.g., 10% of the time period)), high post-prandial time with glucose levels greater than 180 mg/dl (e.g., greater than a threshold amount of time, such as a fixed amount of time (e.g., 90 minutes) or a variable amount of time (e.g., 20% of the time period)), high average or mean glucose (e.g., greater than a threshold value, such as a fixed value (e.g., 180 mg/dL) or a variable number (e.g., the average or mean value the user has had during the time period over a duration of time, such as 2 weeks)), low time in a range such as 70-180 mg/dL (e.g., less than a threshold amount of time, such as a fixed amount of time (e.g., 90 minutes) or a variable amount of time (e.g., 20% of the time period)), and so forth. In one or more implementations, patterns detected in any of the time periods can be mapped to the post-prandial glucose topic.

The high post-prandial glucose peak topic can be mapped to behavior modification feedback of: 1) try to keep your post-prandial glucose lower than X by eating food that helps keep your glucose in range (e.g., low carb), 2) annotate what caused elevated (higher than X) post-prandial glucose levels (e.g., type of food, behavior), 3) try to be active after meals to help keep your glucose in range, e.g., for X days next week (or for X days in a row), be active (e.g., try adding a 15 min walk) after meals (e.g., breakfast/lunch/dinner) to control glucose and reduce spikes, and so forth.

By way of another example, one topic of behavior modifications is A1C—GMI (glucose management indicator) or simply GMI. Patterns that can be mapped to this topic include high average or mean glucose (e.g., greater than a threshold value, such as a fixed value (e.g., 180 mg/dL) or a variable number (e.g., the average or mean value the user has had during the time period over a duration of time, such as 2 weeks)). In one or more implementations, patterns detected in the after breakfast time period, after lunch time period, and after dinner time period can be mapped to the A1C—GMI topic.

The A1C—GMI topic can be mapped to behavior modification feedback of: 1) lower average glucose by X, 2) remember to take your medications as prescribed, talk to your doctor, 3) annotate emotions/stress when occurring, 4) try to be more active during the day (e.g., physical activity goal such as aim at completing X steps next week, aim at exercising for X hours next week, perform physical activity X times next week (e.g., walking, cycling, dancing, climbing stairs, jogging, etc.), and so forth.

By way of another example, one topic of behavior modifications is overnight glucose. Patterns that can be mapped to this topic include high average or mean nocturnal glucose (e.g., greater than a threshold value, such as a fixed value (e.g., 180 mg/dL) or a variable number (e.g., the highest value the user has had during the time period over a duration of time, such as 2 weeks)), low nocturnal time in a range such as 70-180 mg/dL (e.g., less than a threshold amount of time, such as a fixed amount of time (e.g., 30 minutes) or a variable amount of time (e.g., 10% of the time period)), low nocturnal time in a range such as 80-130 mg/dL (e.g., less than a threshold amount of time, such as a fixed amount of time (e.g., 15 minutes) or a variable amount of time (e.g., 5% of the time period)), high nocturnal time in hyperglycemic range (e.g., greater than a threshold amount of time, such as a fixed amount of time (e.g., 30 minutes) or a variable amount of time (e.g., 10% of the time period)), high bedtime glucose (e.g., greater than a threshold value, such as a fixed value (e.g., 250 mg/dL) or a variable number (e.g., the highest value the user has had during the time period over a duration of time, such as 2 weeks)), low bedtime glucose (e.g., less than a threshold value, such as a fixed value (e.g., 70 mg/dL) or a variable number (e.g., the lowest value the user has had during the time period over a duration of time, such as 2 weeks)), high nocturnal time with glucose levels greater than 250 mg/dl (e.g., greater than a threshold amount of time, such as a fixed amount of time (e.g., 30 minutes) or a variable amount of time (e.g., 10% of the time period)), high nocturnal time with glucose levels greater than 180 mg/dl (e.g., greater than a threshold amount of time, such as a fixed amount of time (e.g., 90 minutes) or a variable amount of time (e.g., 20% of the time period)), and so forth. In one or more implementations, patterns detected in the after sleep time period can be mapped to the overnight glucose topic.

The overnight glucose topic can be mapped to behavior modification feedback of: 1) increase your overnight time in range by a X%, 2) remember to take your medications as prescribed, talk to your doctor, 3) try to eat a dinner that won't raise your glucose too high (e.g., smaller portions, fewer carbs), 4) try not to eat close to bedtime (e.g., try not to eat after X PM, set an alarm as a reminder), 5) check your glucose before going to bed to see if you are in range (self-reflection), and so forth.

By way of another example, one topic of behavior modifications is glucose variability. Patterns that can be mapped to this topic include high values for high variability metrics (e.g., less than a threshold number, such as a fixed number (e.g., 2) or a variable number (e.g., the highest value the user has had during the time period over a duration of time, such as 2 weeks)), such as coefficient of variation or time spent in |ROC|>2, and so forth. In one or more implementations, patterns detected in any of the time periods can be mapped to the glucose variability topic.

The glucose variability topic can be mapped to behavior modification feedback of: 1) for X days next week, choose low carbs foods and limit high carb foods, 2) for X days next week, pay attention to how often you have meal-related glucose spikes, 3) try to eat no more than X times during the day, 4) check your glucose before/after a meal to see if you are in range and to understand how specific food impacts your glucose (self-reflection), 5) check and annotate carbs content on foods you eat more often (self-reflection), 6) annotate emotions/stress when occurring next week, and so forth.

By way of another example, one topic of behavior modifications is fasting glucose. Patterns that can be mapped to this topic include high estimated fasting glucose (e.g., greater than a threshold value, such as a fixed value (e.g., 250 mg/dL) or a variable number (e.g., the highest value the user has had during the time period over a duration of time, such as 2 weeks)). In one or more implementations, patterns detected at the beginning of the after breakfast time period and the ending of the sleep time period can be mapped to the fasting glucose topic. The fasting glucose topic can be mapped to behavior modification feedback of: 1) try to eat a dinner that won't raise your glucose too high (smaller portions, fewer carbs), 2) pay attention to how many hours you leave between your last and first meals, 3) try to leave X hours between dinner and breakfast, and so forth.

By way of another example, one topic of behavior modifications is hyperglycemia (also referred to as sustained hyperglycemia). Patterns that can be mapped to this topic include high time greater than 180 mg/dl (e.g., greater than a threshold amount of time, such as a fixed amount of time (e.g., 30 minutes) or a variable amount of time (e.g., 10% of the time period)), high time greater than 250 mg/dl (e.g., greater than a threshold amount of time, such as a fixed amount of time (e.g., 10 minutes) or a variable amount of time (e.g., 3% of the time period)), and so forth. In one or more implementations, patterns detected in the after breakfast time period, after lunch time period, and after dinner time period can be mapped to the hyperglycemia topic.

The hyperglycemia topic can be mapped to behavior modification feedback of: 1) if high time is greater than 15% talk to your doctor, 2) remember to take your medications as prescribed, 3) annotate emotions/stress when occurring next week, 4) try to be more active during the day (physical activity), e.g., aim at completing X steps next week, aim at exercising for X hours next week, perform physical activity X times next week (e.g., walking, cycling, dancing, climbing stairs, jogging, etc.), and so forth.

By way of another example, one topic of behavior modifications is time in range. Patterns that can be mapped to this topic include low time in a range such as 70-180 mg/dL (e.g., less than a threshold amount of time, such as a fixed amount of time (e.g., 90 minutes) or a variable amount of time (e.g., 20% of the time period)). In one or more implementations, patterns detected in the after breakfast time period, after lunch time period, and after dinner time period can be mapped to the time in range topic. The time in range topic can be mapped to behavior modification feedback of: increase time in range by X, and so forth.

By way of another example, one topic of behavior modifications is hypoglycemia. Patterns that can be mapped to this topic include high time (e.g., greater than a threshold amount of time, such as a fixed amount of time (e.g., 30 minutes) or a variable amount of time (e.g., 10% of the time period)) in a hypoglycemic range (e.g., less than 70 mg/dL). In one or more implementations, patterns detected in any of the time periods can be mapped to the hypoglycemia topic. The hypoglycemia topic can be mapped to behavior modification feedback of: 1) talk to your doctor, 2) consider these suggestions (education content that could be added in the message to the user), such as do you know the rule of 15 when you're less than 70, check your glucose before you are physically active, check your glucose before you drive, and so forth.

In one or more implementations, patterns detected in any of the time periods can be mapped to a topic. Additionally or alternatively, patterns detected in only certain time periods may be mapped to a topic. For example, patterns mapped to the fasting glucose topic may be detected at the end of the sleep time period or the beginning of the after breakfast time period, but not during other time periods. By way of another example, patterns mapped to the overnight glucose topic may be detected during the sleep time period but not during other time periods.

In one or more implementations, some patterns have a one-to-one mapping to topics. For example, the high estimated fasting glucose pattern is mapped to just the fasting glucose topic. However, other patterns may potentially map to multiple topics. For example, the high time greater than 180 mg/dl pattern may be mapped to the post-prandial glucose topic or the hyperglycemia topic. For such patterns, the mapping module 1510 determines which topic to map the pattern to based on how many time periods the pattern is identified in.

For example, if one or both of the high post-prandial time with glucose levels greater than 180 mg/dl or high post-prandial time with glucose levels greater than 250 mg/dl patterns are detected in less than a threshold number of time periods in a day or other 24-hour period (e.g., 3 time periods), then the pattern is mapped to the post-prandial glucose topic. However, if the patterns are detected in at least the threshold number of time periods in a day or 24-hour period (e.g., at least 3 time periods), then the pattern is mapped to the hyperglycemia topic.

By way of another example, if the high average or mean glucose pattern is detected in less than a threshold number of time periods in a day or other 24-hour period (e.g., 3 time periods), then the pattern is mapped to the post-prandial glucose topic. However, if the pattern is detected in at least the threshold number of time periods in a day or 24-hour period (e.g., at least 3 time periods), then the pattern is mapped to the GMI topic.

By way of another example, if the low time in a range such as 70-180 mg/dL pattern is detected in less than a threshold number of time periods in a day or other 24-hour period (e.g., 3 time periods), then the pattern is mapped to the post-prandial glucose topic. However, if the pattern is detected in at least the threshold number of time periods in a day or 24-hour period (e.g., at least 3 time periods), then the pattern is mapped to the time in range topic.

The mapping module 1510 maps multiple patterns to the same topic to reduce redundancy in situations in which the same behavior modification feedback could be provided to improve diabetes management. For example, the same behavior modification feedback could be provided to improve diabetes management in situations in which a pattern of high post-prandial glucose peak after lunch and a pattern of high post-prandial time with glucose levels greater than 250 mg/dl after lunch are detected. By mapping both of these patterns to the “post-prandial glucose” topic, the behavior modification identification system 308 can avoid providing the same behavior modification feedback if both patterns are detected in a time period.

Various different example times, glucose levels, and other values are discussed with reference to the detected patterns 1528. It should be noted that these various different times, glucose levels, and other values are just examples and that various other times, glucose levels, and other values can be used instead.

The mapping module 1510 outputs one or more topics 1532 to the behavior modification selection module 1512. The one or more topics 1532 include each topic that a detected pattern 1528 is mapped to. In situations in which multiple patterns map to the same topic, the one or more topics 1532 need include (and typically does include) that topic only once. However, the one or more topics 1532 may include the same topic for different time periods, such as in situations in which a pattern mapped to the same topic in multiple different time periods. In one or more implementations, for each topic 1532, the mapping module 1510 also provides one or both of the detected patterns 1528 that mapped to the topic 1532 and the normalized features 1530.

The various topics to which patterns are mapped correspond to (are mapped to) one or more behavior modification feedback. The behavior modification selection module 1512 receives the one or more topics 1532 (and optionally the normalized features 1530) and selects behavior modification feedback from the behavior library 1538 to provide to the UI module 1514 (or the feedback presentation system 122) for output. In one or more embodiments, the behavior modification selection module 1512 maps each topic 1532 to particular behavior modification feedback (e.g., a particular message or text). Each of the behavior modification feedback in the behavior library 1538 is also referred to as being mapped to a topic 1532. The mappings between topics 1532 and behavior modification feedback can be specified in various manners, such as by a developer or designer of the behavior modification identification system 308, by a health care provider or professional, and so forth.

The behavior modification feedback in the behavior library 1538 can be obtained from any of a variety of sources. For example, the behavior modification feedback can be obtained from health care providers or professionals, a clinician, standard of care or other publications, and so forth. In one or more implementations, the behavior library 1538 includes user input or specified behavior modification feedback, allowing the user to select or create behavior modification feedback that they would like to see if the pattern that maps to their behavior modification feedback is detected. The behavior modification feedback also optionally includes additional educational material or links to resources (e.g., via the Internet) for additional information describing the behavior modification feedback, describing terms in the behavior modification feedback, and so forth. E.g., if a behavior modification feedback is to try to eat a dinner with fewer carbs, the behavior modification feedback can include links to guides identifying foods or recipes that are low carb.

In one or more implementations, the behavior modification selection module 1512 selects all behavior modification feedback that is mapped to by at least one topic 1532 to provide to UI module 1514 (or the feedback presentation system 122).

Additionally or alternatively, in situations in which multiple behavior modification feedback is mapped to by different topics, behavior modification selection module 1512 selects one or more of the mapped to behavior modification feedback to provide to UI module 1514 (or the feedback presentation system 122). The behavior modification selection module 1512 can select one or more of the mapped to behavior modification feedback in various manners, such as randomly or pseudorandomly selecting one of the mapped to mapped to behavior modification feedback. Additionally or alternatively, the behavior modification selection module 1512 can prioritize the multiple mapped to behavior modification feedback and select one or more of the multiple mapped to behavior modification feedback a highest priority (or priorities). For example, the mapped to behavior modification feedback having the highest priority is selected.

The behavior modification selection module 1512 optionally uses various criteria to determine which of the multiple mapped to behavior modification feedback to select. These criteria can be based on various factors, such as how recently the pattern that mapped to a topic was detected, a ranking or prioritization of behavior modification feedback, topics, or categories of behavior modification feedback, and so forth. For example, the patterns corresponding to the normalized features 1530 have various sizes as discussed above. Accordingly, the behavior modification feedback mapped to by a topic to which the pattern having the largest size is mapped is selected.

By way of another example, behavior modification feedback mapped to by a topic to which a pattern that was detected less recently is mapped is selected over behavior modification feedback mapped to by a topic to which a pattern that was detected more recently is mapped. E.g., this allows behavior modification feedback mapped to by different topics to be selected as behavior modification feedback 1534 and avoids repeating behavior modification feedback too frequently.

By way of another example, behavior modification feedback corresponding to certain topics or categories can be selected over behavior modification feedback corresponding to other topics or categories. For example, behavior modification feedback mapped to by a hypoglycemia topic may be selected over behavior modification feedback mapped to by an engagement with a glucose monitoring application topic. E.g., this allows behavior modification feedback mapped to by topics or categories deemed more important to the user's health to be selected before behavior modification feedback mapped to by topics or categories deemed less important.

By way of another example, behavior modification feedback designated (e.g., by a developer or designer of the behavior modification selection module 1512) to be more urgent or safety-related is selected over behavior modification feedback that is less urgent or safety-related. E.g., this allows behavior modification feedback corresponding to urgent or safety-related features (e.g., not staying within ranges or exceeding threshold glucose levels) to be selected over other non-urgent or non-safety-related behavior modification feedback and display or otherwise present more critical behavior modification feedback to the user.

By way of another example, behavior modification feedback designated as being higher priority (e.g., by the user 102) is selected over behavior modification feedback that is designated as being lower priority (e.g., by the user 102). E.g., this allows behavior modification feedback that is of greater interest to the user to be displayed or otherwise presented rather than behavior modification feedback that is of less interest to the user.

By way of another example, behavior modification feedback designated as being helpful by the user 102 or associated with an improvement in diabetes management is selected over behavior modification feedback that is not designated as being helpful by the user 102 or did not lead to an improvement in diabetes management. E.g., this allows behavior modification feedback that is more helpful to the user, or that previously resulted in an improvement in diabetes management, to be presented to the user again (optionally customized with updated values, such as walk 4 times per week rather than 2 times per week) rather than other behavior modification feedback.

Furthermore, the behavior modification selection module 1512 can receive additional data 1524, which can be any additional data that may be used to identify poor diabetes management as discussed above. The additional data 1524 may include data from various sources, for example applications or programs of the computing device 106, user input by the user 102, input by a healthcare provider (e.g., the user's doctor or nurse), external devices such as activity trackers, and so forth.

The additional data 1524 can include data that relates to user interactions with the computing device 106, with the display of the computing device 106, or with other system components that indicate level of engagement with diabetes management as discussed above.

By way of another example, additional data 1524 can include activity data, such as a number of steps walked over a particular range of time (e.g., every 10 seconds, every minute), heart rate over a particular range of time (e.g., at regular or irregular intervals, such as every 15 seconds) with timestamps, speed of movement with timestamp (e.g., at regular or irregular intervals, such as every 15 seconds), and so forth. Activity data can be received from various sources, such as wearable glucose monitoring device 104, an activity tracking application running on computing device 106, an activity or fitness tracker worn by the user 102, and so forth.

By way of another example, additional data 1524 can include data regarding sleeping patterns of the user. E.g., additional data 1524 can include data indicating times when the user is sleeping, the sleep state (e.g., Stage 1, Stage 2, Stage 3, or rapid eye movement (REM) sleep) of the user at particular times, and so forth.

By way of another example, additional data 1524 can include data regarding user engagement with others of user population 108, such as via glucose monitoring platform 110. E.g., this other-user engagement data can include timestamps of when the user 102 communicated with another user as well as who that other user was, descriptions of what information was communicated with another user, and so forth.

By way of another example, additional data 1524 can include meal data. E.g., this meal data can include timestamps of when the user 102 ate and what foods were consumed, timestamps of when particular types or classes of foods were consumed (e.g., vegetables, grain, meat, sweets, soda), amounts of food consumed, and so forth.

By way of another example, additional data 1524 can include sleep data, such as data indicating minutes of the day when the user was sleeping. Sleep data can be received from various sources, such as wearable glucose monitoring device 104, a sleep tracking application running on computing device 106, an activity or fitness tracker worn by the user 102, and so forth.

By way of another example, additional data 1524 can include medication data. E.g., this medication data can include timestamps of when user 102 took medicine (e.g., basal insulin) and what medicine was taken (which can be used to determine whether the user 102 is taking his or her medicine at the prescribed times or intervals), indications of changes in medicines (e.g., changes in types or dosages of medicines taken), and so forth.

By way of another example, additional data 1524 can include data that reflects stress management, such as heart rate variability (HRV), skin conductivity and temperature, respiration rate measurements, data from an electroencephalogram (EEG), cortisol in biofluids, volatile organic components (VOCs) emitted from the skin, and so forth.

By way of another example, additional data 1524 can include current health data. E.g., this current health data can include whether a user is currently sick (e.g., has a cold, has a virus), whether a user is currently recovering from an operation or other procedure, diseases or chronic conditions that the user is currently diagnosed with (e.g., kidney disease or liver disease), and so forth.

In one or more implementations, the behavior modification selection module 1512 can select one or more of the mapped to behavior modification feedback based on the additional data 1524, such as by using the additional data 1524 to prioritize behavior modification feedback or filter out behavior modification feedback. For example, the behavior modification selection module 1512 would filter out (not select) behavior modification feedback to perform physical activity X times next week if the additional data 1524 indicates the user is sick or recovering from foot surgery. By way of another example, the behavior modification selection module 1512 would filter out (not select) behavior modification feedback to try to be active after meals to help keep your glucose in range if the additional data 1524 indicates the user is regularly active after meals. By way of another example, the behavior modification selection module 1512 could select or give a higher priority to behavior modification feedback to try to be active after meals to help keep your glucose in range if the additional data 1524 indicates the user is rarely (or never) active after meals.

In one or more implementations, the behavior modification selection module 1512 communicates with a behavior modification feedback customization module 1536. Some behavior modification feedback includes variables or blanks that are altered based on the particular user 102. The behavior modification feedback customization module 1536 receives one or more of the glucose measurements 114, the grouped measurements 1520, the features 1522 and the additional data 1524, and alters or fills in these variables or blanks in the behavior modification feedback to customize the glucose measurement feedback to the user 102. For example, various different behavior modification feedback discussed above include X, such as check your glucose X number of times per day or try to keep your post-prandial glucose lower than X by eating food that helps keep your glucose in range (e.g., low carb). The behavior modification feedback customization module 1536 determines a value (e.g., a specific number or range of numbers) to replace the X with so that the behavior modification feedback 1534 displayed to the user is “keep your post-prandial glucose lower than 197 by eating food that helps keep your glucose in range (e.g., low carb)” rather than simply “keep your post-prandial glucose lower by eating food that helps keep your glucose in range (e.g., low carb)” or replacing the X with a standard value (e.g., 180).

The behavior modification feedback customization module 1536 customizes behavior modification feedback customization module 1536 in various manners. In one or more implementations, the behavior modification feedback customization module 1536 adds a default value (e.g., 50) to a glucose measurement 114 or a feature 1522. For example, a feature 1522 may be the mean glucose measurement 114 at the beginning of corresponding time periods (e.g., dinner time periods). The behavior modification feedback customization module 1536 adds the default value (e.g., 50) to the mean value (e.g., 147), resulting in the customized behavior modification feedback of “keep your post-prandial glucose lower than 197 by eating food that helps keep your glucose in range (e.g., low carb).”

Additionally or alternatively, the behavior modification feedback customization module 1536 analyzes the various data it receives to determine a realistic, actionable goal for the user 102. For example, if the user does not regularly walk after meals, the behavior modification feedback customization module 1536 can determine to customize behavior modification feedback to suggest walking two times per week after meals. However, if the user regularly walks two times per week after meals, the behavior modification feedback customization module 1536 can determine to customize behavior modification feedback to suggest walking four times per week after meals. By way of another example, if the user does not check their glucose level via glucose monitoring application 116 each day, the behavior modification feedback customization module 1536 can determine to customize behavior modification feedback to suggest “check your glucose 3 times per day”. However, if the user regularly checks their glucose level via glucose monitoring application 116 two times each day, the behavior modification feedback customization module 1536 can determine to customize behavior modification feedback to suggest “check your glucose 6 times per day”.

The UI module 1514 optionally receives the selected behavior modification feedback 1534 and causes the behavior modification feedback 1534 to be displayed or otherwise presented (e.g., at computing device 106). This display or other presentation can take various forms, such as a static text display, graphic or video display, audio presentation, combinations thereof, and so forth. In one or more implementations, different topics or categories of behavior modification feedback are displayed or otherwise presented in different manners. For example, behavior modification feedback corresponding to different topics or categories can be displayed using different colors, different icons, and so forth. The example 1600 of FIG. 16 illustrates an example of behavior modification feedback as behavior modification feedback 1612.

The behavior modification identification system 308 generates and displays or otherwise communicates the selected behavior modification feedback 1534 at various intervals. In one or more embodiments, the behavior modification feedback 1534 is generated and displayed or otherwise communicated weekly, such as Sunday evening so that the behavior modification feedback 1534 is available to the user at the beginning of the week (e.g., giving the user a goal to achieve for the week). Additionally or alternatively, other timings can be used, such as bi-weekly, daily, bi-daily, and so forth. Additionally or alternatively, the behavior modification selection module 1512 may display or otherwise communicate high priority behavior modification feedback 1534 immediately, such as in situations where there is an immediate safety risk (e.g., due to hypoglycemia).

In one or more implementations, the behavior modification selection module 1512 tracks the behavior modification feedback 1534 provided to the UI module 1514, determines whether the behavior modification feedback 1534 was followed, and provides additional behavior modification feedback 1534 based on whether the behavior modification feedback 1534 was followed. For example, if the behavior modification feedback 1534 is to complete 35,000 steps next week, the additional data 1524 can include activity data indicating whether the user completed 35,000 steps over the week. E.g., behavior modification feedback congratulating the user on successfully following the previous week's behavior modification feedback may be provided if the user completed 35,000 steps, or behavior modification feedback encouraging the user to keep up the good work if they did not complete 35,000 steps but came close or had significant improvement over previous weeks.

The behavior modification identification system 308 optionally takes additional actions based on the behavior modification feedback 1534. In one or more implementations, these actions include notifying the glucose monitoring application 116 or the wearable glucose monitoring device 104 that the frequency with which glucose measurements 114 are produced can be reduced. For example, if the behavior modification identification system 308 identifies that no patterns are detected for particular time periods (e.g., corresponding to sleep), the behavior modification identification system 308 notifies the glucose monitoring application 116 or wearable glucose monitoring device 104 that the frequency with which glucose measurements 114 are produced can be reduced (e.g., from every 5 minutes to every 10 minutes), reducing the power expended to produce glucose measurements 114.

Additionally or alternatively, these actions include determining whether to recommend ongoing CGM use (e.g., starting a new sensor immediately after the current sensor expires) or whether it may be appropriate to take a break from using CGM and starting a new sensor at some later date. For example, if the behavior modification identification system 308 identifies that patterns are detected regularly in all time periods, the behavior modification identification system 308 recommends (e.g., via display or other presentation to the user) ongoing CGM use.

Discussions are also included herein with reference to behavior modification feedback being displayed or otherwise presented to the user 102. Additionally or alternatively, the behavior modification feedback is communicated to or otherwise delivered to others, such as a clinician (e.g., the user's primary care physician or nurse), a pharmacist, and so forth. This can serve to partially automate some of the manual effort of reviewing raw glucose or other diabetes management data that a clinician may have to do on their own in the absence of generated behavior modification feedback. Additionally or alternatively, rather than providing the behavior modification feedback 1534, the behavior modification selection module 1512 can provide the features 1522, normalized features 1530, or detected patterns 1528 may be provided to the clinician, pharmacist, or others, enabling them to apply their own preferred behavior modification selection (if any) in determining which behavior modification feedback should be passed along to the user 102.

Discussions are also included herein with reference to determining particular time periods within the time window. These time periods can be determined prior to analysis of the features 1522 by the pattern detection module 1506 to detect patterns in corresponding time periods of the time window. Additionally or alternatively, these time periods may be determined at a later time. In one or more implementations, the pattern detection module 1506 or another module may analyze the features 1522 in various time ranges within the day (e.g., 30-minute, 60-minute, 120-minute, etc. ranges of time at some interval such as 5 or 10 minutes). If the pattern detection module 1506 detects a pattern in one of those time ranges on a single day, that time range is treated by the behavior modification identification system 308 as a time period. The time range is optionally expanded (e.g., by 10 minutes on either side) to create the time period. The corresponding time periods in other time windows (e.g., the same time range in other days) are then used to determine whether there is a pattern in the corresponding time periods across multiple time windows.

For example, assume the time window is one day. The pattern detection module 1506 may begin analyzing the features 1522 over the previous 60 minutes beginning at 1:00 am on a particular day, moving forward in 10 minute intervals. When analyzing the features 1522 for the time range of 1:20 am-2:20 am, the pattern detection module 1506 may detect a pattern in the time range of 1:20 am-2:20 am. The pattern detection module 1506 uses the time range of 1:20 am-2:20 am (or expands the time range to 1:10 am-2:30 am) as a time period and analyzes the features 1522 for that time period across multiple days (e.g., the previous week) to detect whether there is a pattern in the corresponding time periods of the multiple days.

Additionally or alternatively, in one or more implementations the behavior modification identification system 308 (e.g., the behavior modification selection module 1512) maintains a record of one or more of detected patterns 1528, features 1522, and behavior modification feedback 1534. The behavior modification identification system 308 (e.g., the behavior modification selection module 1512) analyzes the detected patterns 1528 or features 1522 over longer ranges of time, such as months or years, and identifies improvements over those longer ranges of time. For example, the behavior modification identification system 308 compares the detected patterns 1528 or features 1522 for a current 1-week time window to the detected patterns 1528 or features 1522 of a 1-week time window six months or a year ago. Improvements in diabetes management identified by this comparison (e.g., as indicated by the features 1522 or by patterns detected six months or a year ago that are not detected in the current week) can be identified to the user via UI module 1514. E.g., a congratulatory message identifying the improvement may be communicated, displayed, or otherwise presented to the user or other person (e.g., health care provider or clinician). The behavior modification feedback that was previously provided to the user (e.g., six months or a year ago) can also be communicated, displayed, or otherwise presented to the user or other person, providing an indication of what behavior modification feedback was followed by the user that resulted in the improvement in diabetes management.

Discussions are also included herein with reference to detecting patterns, mapping patterns to topics, and mapping topics to behavior modification feedback. Additionally or alternatively, the techniques discussed herein need not use topics. In such situations detected patterns can be mapped to behavior modification feedback. Which patterns map to which behavior modification feedback can be specified in various manners, such as by a developer or designer of the behavior modification identification system, by a health care provider or professional, and so forth.

It should be noted that, as discussed above, the behavior modification feedback 1534 can be provided to the feedback presentation system 122 as feedback indications 312. In such situations the behavior modification identification system 308 need not include the UI module 1514. Additionally or alternatively, the topics 1532, normalized features 1530, additional data 1524, can be provided to the feedback presentation system 122 as feedback indications 312. In such situations the feedback presentation system 122 identifies feedback to be provided to the user (or others, such as a clinician or pharmacist), optionally using the behavior library 1538 and the behavior modification feedback customization module 1536, as discussed in more detail below. The feedback presentation system 122 optionally identifies behavior modification feedback to be provided to the user using any one or more of the techniques discussed herein with respect to the behavior modification selection module 1512.

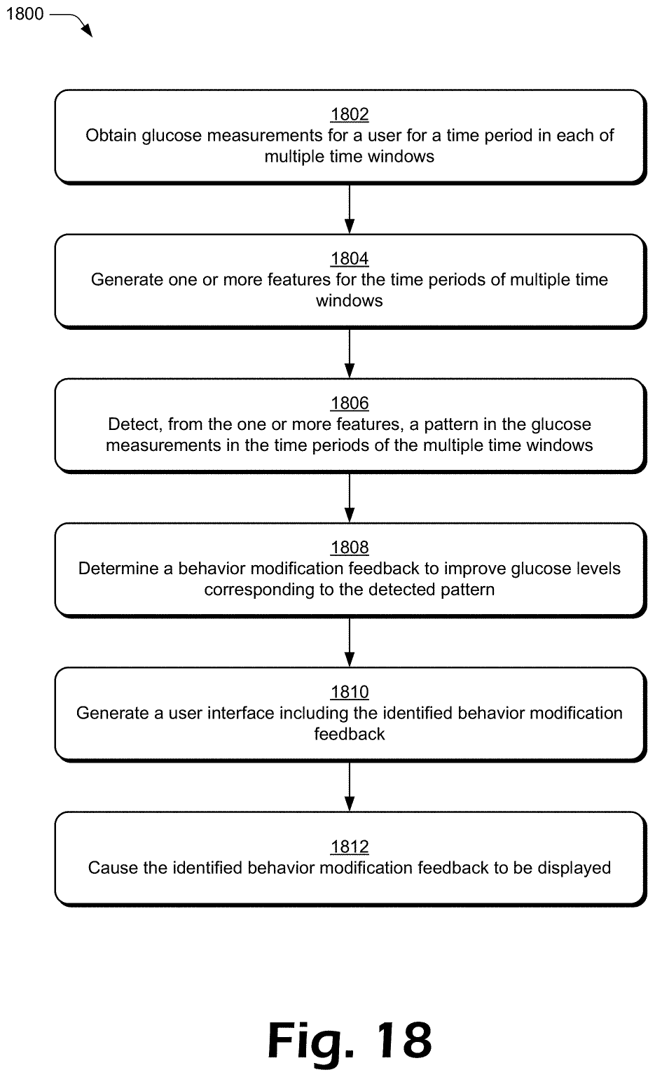

FIG. 18 describes an example procedure 1800 for implementing behavior modification feedback for improving diabetes management. Aspects of the procedures may be implemented in hardware, firmware, or software, or a combination thereof The procedures are shown as a set of blocks that specify operations performed by one or more devices and are not necessarily limited to the orders shown for performing the operations by the respective blocks. Procedure 1800 is performed, for example, by a behavior modification identification system, such as the behavior modification identification system 308 and optionally in part by a feedback presentation system, such as the feedback presentation system 122. Procedure 1800 is performed, for example, by a behavior modification identification system, such as the behavior modification identification system 308.

Glucose measurements for a user for a time period in each of multiple time windows are obtained (block 1802). The glucose measurements are obtained for corresponding time periods across the multiple time windows, such as for the lunch time periods on multiple days. These glucose measurements are obtained from a glucose sensor of, for example, a continuous glucose level monitoring system with the glucose sensor being inserted at an insertion site of the user.

One or more features for the time periods of the multiple time windows are generated (block 1804). These one or more features are generated from the glucose measurements.

A pattern in the glucose measurements in the time periods of the multiple time windows is detected (block 1806). This detection is made based on the generated features for the time periods of the multiple time windows.

A behavior modification feedback to improve glucose levels corresponding to the detected pattern is determined (block 1808). The detected pattern may be mapped to a topic that is mapped to one or more behavior modification feedback, one or more of which is selected in 608. Additionally or alternatively, the detected pattern may be mapped to or correspond to multiple behavior modification feedback, and one or more of the multiple behavior modification feedback is selected in block 1808.

A user interface including the identified behavior modification feedback is generated (block 1810). The identified diabetes management feedback is caused to be displayed (block 1812) or otherwise presented. Additionally or alternatively, the identified diabetes management feedback can be communicated to or otherwise presented to a clinician, pharmacist, other health care provider, and so forth.

### Glucose Prediction System Architecture

Generally, a glucose prediction system 310 receives a data stream of glucose measurements. Various other data streams are also received, such as activity data (e.g., number of steps taken by the user). A glucose prediction system analyzes, for example, activity data of a user and determines when a bout of physical activity occurs. The glucose prediction system predicts what the glucose measurements of the user would have been had the physical activity not occurred, and takes various actions based on the predicted glucose measurements (e.g., provides feedback to the user indicating what their glucose would have been had they not engaged in the physical activity).

Additionally or alternatively, the received data streams include various other data that indicates events or conditions that may affect glucose of the user, such as activities the user engages in, behaviors of the user, reactions of the user, medical conditions of the user, biological data of the user, and so forth. The glucose prediction system analyzes this data to identify such events or conditions, and predicts what the glucose measurements of the user would have been had the identified events or conditions not occurred or not been present. The glucose prediction system takes various actions based on the predicted glucose measurements (e.g., provides feedback to the user indicating what their glucose would have been had the identified events or conditions not occurred or not been present).

The techniques discussed herein apply analogously to determining when a period of physical activity does not occur (or other events or conditions do not occur or are not present). The glucose prediction system predicts what the glucose measurements of the user would have been had the physical activity occurred (or other events or conditions occurred or been present), and takes various actions based on the predicted glucose measurements (e.g., provides feedback to the user indicating what their glucose would have been had they engaged in the physical activity or if other events or conditions had occurred or been present).

The techniques discussed herein predict or estimate what glucose measurements would have been for a user if particular events or conditions had occurred or been present (or had not occurred or been present). Feedback giving positive reinforcement of one or both of healthy glucose management behavior modifications and patient-specific goals (e.g., using activity to mitigate post-prandial spikes or lower blood glucose after sustained hyperglycemia) is provided. This helps the user improve diabetes management and his overall health.

Furthermore, the techniques discussed herein provide real-time teachable moments by linking specific behavior modifications to improved diabetes management outcomes. The user receives real-time feedback allowing the user to know that his behavior or choices have had a positive impact on his glucose, allowing him to continue such behavior in the future and improve his overall health.

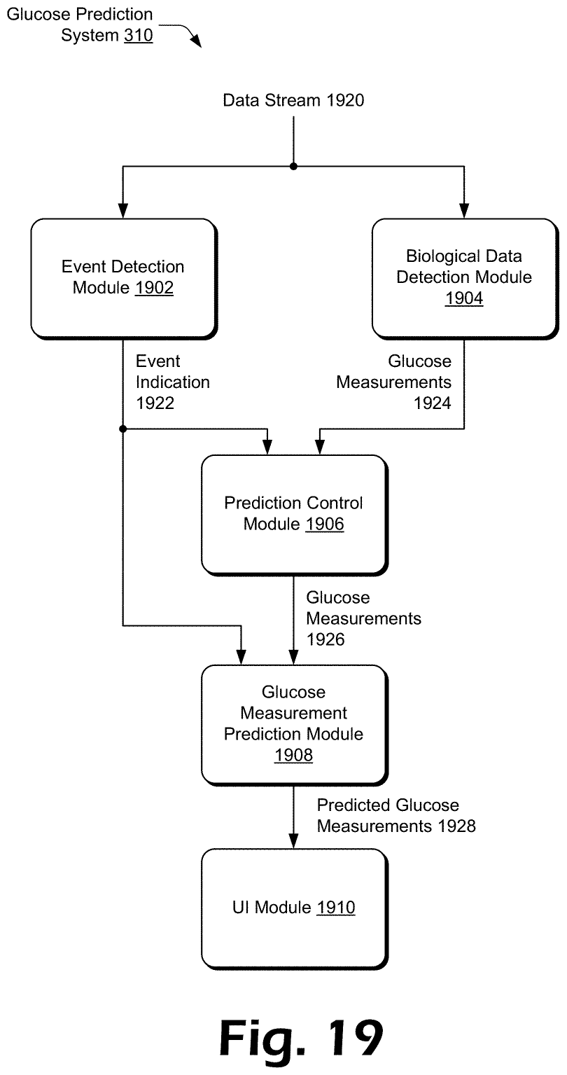

FIG. 19 is an illustration of an example architecture of a glucose prediction system 310. The glucose prediction system 310 includes an event detection module 1902, a biological data detection module 1904, a prediction control module 1906, a glucose measurement prediction module 1908, and a UI module 1910 (optional). Generally, the glucose prediction system 310 analyzes activity data of a user and determines when a period of physical activity occurs. The glucose prediction system 310 predicts what the glucose measurements of the user 102 would have been had the physical activity not occurred, and takes various actions based on the predicted glucose measurements (e.g., provides feedback to the user, optionally in conjunction with the feedback presentation system 122, indicating what their glucose would have been had they not engaged in the physical activity).

The event detection module 1902 and biological data detection module 1904 each receive a data stream 1920 (e.g., the glucose measurements 114 and the additional data 302 of FIG. 3). The data in the data stream 1920 can be received from various different sources, such as the wearable glucose monitoring device 104, one or more sensors of the computing device 106, another sensor or device worn by the user 102, user inputs (e.g., specifying times when particular activities occurred or actions were taken by the user, specifying measurements received from various sensors), a local or remote database (e.g., accessed via the network 112), and so forth. The data in the data stream 1920 can include data received at regular intervals (e.g., approximately every 5 minutes), single occurrence data (e.g., data input via a user interface, such as data describing a meal eaten at a particular time), and so forth. In one or more implementations, the data stream 1920 includes glucose measurements 114 and timestamps indicating when each of the glucose measurements 114 was taken (e.g., by wearable glucose monitoring device 104) or received (e.g., by glucose monitoring application 116). The timestamp may be provided, for example, by the wearable glucose monitoring device 104 or the glucose monitoring application 116. Additionally or alternatively, the data stream 1920 includes any of a variety of other data that indicates events or conditions that may affect glucose of the user 102 (e.g., glucose levels of the user 102), such as activities the user 102 engages in, behaviors of the user 102, reactions of the user 102, medical conditions of the user 102, biological data of the user 102, and so forth.

In one or more implementations, the data stream 1920 includes physical activity data, such as a number of steps walked over a particular range of time (e.g., every 10 seconds, every minute), heart rate over a particular range of time (e.g., at regular or irregular intervals, such as every 15 seconds) with timestamps, speed of movement with timestamp (e.g., at regular or irregular intervals, such as every 15 seconds), raw or filtered accelerometer data, and so forth. Physical activity data can be received from various sources, such as wearable glucose monitoring device 104, an activity tracking application running on computing device 106, an activity or fitness tracker worn by the user 102, and so forth.

Additionally or alternatively, data stream 1920 includes meal data. E.g., this meal data can include timestamps of when the user 102 ate and what foods were consumed, timestamps of when particular types or classes of foods were consumed (e.g., vegetables, grain, meat, sweets, soda), amounts of food consumed, and so forth.

Additionally or alternatively, data stream 1920 includes sleep data, such as data indicating minutes of the day when the user was sleeping. Sleep data can also include data regarding sleeping patterns of the user. E.g., data stream 1920 can include data indicating times when the user is sleeping, the sleep state (e.g., Stage 1, Stage 2, Stage 3, or rapid eye movement (REM) sleep) of the user at particular times, and so forth. Sleep data can be received from various sources, such as wearable glucose monitoring device 104, a sleep tracking application running on computing device 106, an activity or fitness tracker worn by the user 102, and so forth.

Additionally or alternatively, data stream 1920 includes medication data. E.g., this medication data can include timestamps of when user 102 took medicine and what medicine was taken (which can be used to determine whether the user 102 is taking his or her medicine at the prescribed times or intervals), indications of changes in medicines (e.g., changes in types or dosages of medicines taken), and so forth.

Additionally or alternatively, data stream 1920 includes data that reflects stress management, such as heart rate variability (HRV), skin conductivity and temperature, respiration rate measurements, data from an electroencephalogram (EEG), cortisol in biofluids, volatile organic components (VOCs) emitted from the skin, and so forth.

Additionally or alternatively, data stream 1920 includes data regarding user engagement with glucose monitoring application 116. E.g., this application engagement data can include timestamps of when the user 102 viewed the application as well as what screens or portions of the UI were viewed, timestamps of when the user 102 provided input to (or otherwise interacted with) the application 116 as well as what that input was, timestamps of when the user viewed or acknowledged feedback provided by the application 116, and so forth.

Additionally or alternatively, data stream 1920 include data that relates to user interactions with the computing device 106, with display of the computing device 106, or with other system components that indicate level of engagement with diabetes management. Examples of such data include the number of times applications (e.g., glucose monitoring application) is opened, the time spent reviewing glucose data or previous feedback or educational materials, the frequency of interactions with coaches or clinicians, and so forth.

Additionally or alternatively, data stream 1920 includes data regarding user engagement with others of user population 108, such as via glucose monitoring platform 110. E.g., this other-user engagement data can include timestamps of when the user 102 communicated with another user as well as who that other user was, descriptions of what information was communicated with another user, and so forth.

The event detection module 1902 receives data stream 1920 and identifies events or conditions in the data stream 1920 that may affect glucose levels of the user. These events or conditions can be any event or condition indicated by the data in the data stream 1920, such as physical activity, sleep, meals consumed, medication taken, and so forth. The event detection module 1902 outputs an event indication 1922 that identifies these events or conditions, such as an indication of a bout of physical activity by the user 102, an indication of a time the user 102 was sleeping, an indication of meals consumed by the user 102, an indication of medication taken by the user 102, and so forth.

In one or more implementations, the event detection module 1902 receives physical activity data in data stream 1920 and identifies bouts of physical activity by the user 102. Physical activity refers to any bodily movement produced by skeletal muscle that results in energy expenditure above resting (basal) levels. The event detection module 1902 identifies a bout of physical activity, which is an amount of time during which the energy expenditure by the user is at least a threshold amount above resting levels. The event detection module 1902 identifies bouts of physical activity in any of a variety of different manners. In one or more implementations, the event detection module 1902 identifies a bout of physical activity based on a number of steps taken. For example, a bout of physical activity is user 102 taking at least a threshold number of steps (e.g., 60) per minute for at least a threshold amount of time (e.g., 5 minutes) and without dropping below the threshold number of steps (e.g., 60) for at least a consecutive amount of time (e.g., 5 minutes). The bout ends when the user 102 drops below the threshold number of steps (e.g., 60) for at least the consecutive number of minutes (e.g., 5 minutes). Allowing the number of steps to drop below the threshold number of steps for less than the consecutive amount of time allows a single bout of physical activity to be identified even though the user takes small resting breaks during the physical activity. These thresholds (e.g., threshold amount of time or threshold number of steps) are optionally adjusted or modified based on various characteristics of the user such as their age, fitness level, prevalence of co-morbidities that may affect walking gate speed, and so forth. E.g., Older individuals may require more conservative thresholds to reach the same intensity as a younger individual with higher thresholds.

Additionally or alternatively, the event detection module 1902 identifies a bout of physical activity based on any of various heart-rate based intensity values. One such heart-rate based intensity value is a percent heart rate reserve value for user 102. The percent heart rate reserve value indicates how close the user is to their estimated max heart rate. For example, the percent heart rate reserve (% HHR) value for a user at a current time can be identified as:

$$\% HHR = \frac{{HR}_{ex} - {HR}_{rest}}{HRR} * 100$$

where HRₑₓ refers to the heart rate of the user at the current time, HRᵣₑₛₜ refers to the resting heart rate of the user, and HRR refers to the heart rate reserve of the user 102, which is determined as HRR=HRₘₐₓ−HRᵣₑₛₜ, where HRₘₐₓ refers to the max heart rate of the user.

The current heart rate of a user is obtained in various manners, such as from an activity monitor worn by the user. The resting heart rate of the user is obtained in various manners, such as from an activity monitor worn by the user, input from the user via a UI (e.g., of computing device 106), and so forth. The max heart rate of the user is obtained in various manners, such as from a VO₂ max test, estimated from various formulas, and so forth.

The event detection module 1902 uses the percent heart rate reserve value in various manners to determine a bout of physical activity for the user 102. For example, a bout of physical activity is the percent heart rate reserve value for the user 102 exceeding a threshold amount (e.g., 40%) for at least a threshold amount of time (e.g., 3 minutes) and without dropping below the threshold amount (e.g., 40%) for at least a consecutive amount of time (e.g., 3 minutes). The bout ends when the user 102 drops below the threshold amount (e.g., 40%) for at least the consecutive amount of time (e.g., 3 minutes). Allowing the percent heart rate reserve to drop below the threshold amount for less than the consecutive amount of time allows a single bout of physical activity to be identified even though the user takes small resting breaks during the physical activity.

Another such heart-rate based intensity value is a percent of max heart rate. The max heart rate of the user is obtained in various manners as discussed above. The event detection module 1902 uses the percent of max heart rate in various manners to determine a bout of physical activity for the user 102. For example, a bout of physical activity is the max heart rate for the user 102 exceeding a threshold amount (e.g., 60%) for at least a threshold amount of time (e.g., 3 minutes) and without dropping below the threshold amount (e.g., 60%) for at least a consecutive amount of time (e.g., 3 minutes). The bout ends when the user 102 drops below the threshold amount (e.g., 60%) for at least the consecutive amount of time (e.g., 3 minutes). Allowing the max heart rate to drop below the threshold amount for less than the consecutive amount of time allows a single bout of physical activity to be identified even though the user takes small resting breaks during the physical activity.

Additionally or alternatively, the event detection module 1902 identifies a bout of physical activity based on Metabolic Equivalents (METs) for user 102. METs are an estimate of the amount of energy used relative to the user sitting at rest, and one MET is the amount of oxygen consumed by the user while sitting at rest. The METs expended by a user at any a current time is obtained in various manners, such as from an activity monitor worn by the user.

The event detection module 1902 uses METs in various manners to determine a bout of physical activity for the user 102. For example, a bout of physical activity is the number of METs for the user 102 exceeding a threshold amount (e.g., 2 METs) for at least a threshold amount of time (e.g., 5 minutes) without dropping below the threshold amount (e.g., 2 METs) for at least a consecutive amount of time (e.g., 5 minutes). The bout ends when the user 102 drops below the threshold amount (e.g., 2 METs) for at least the consecutive number of minutes (e.g., 5 minutes). Allowing the METs to drop below the threshold amount for less than the consecutive amount of time allows a single bout of physical activity to be identified even though the user takes small resting breaks during the physical activity.

The event detection module 1902 may also use multiple different techniques concurrently to identify a bout of physical activity. In such situations, the threshold amounts or values may vary from when a single technique is used. For example, a bout of physical activity is the percent heart rate reserve value for the user 102 exceeding a threshold amount (e.g., 45%) and the user 102 taking at least a threshold number of steps (e.g., 40) per minute for at least a threshold amount of time (e.g., 5 minutes). The bout continues for as long as the user 102 does not drop below the threshold amount (e.g., 45% heart rate reserve and 40 steps per minute) for at least a consecutive amount of time (e.g., 5 minutes). The bout ends when the user 102 drops below the threshold amount (45% heart rate reserve and 40 steps per minute) for at least the consecutive amount of time (e.g., 5 minutes). This combination allows, for example, a smaller number of steps to be identified as a bout of physical activity if the user's heart rate is high enough.

Additionally or alternatively, bouts of physical activity can be identified in various other manners. For example, user input (e.g., voice input, gesture, selection of a button on computing device 106) can be received indicating the beginning and the ending of a bout of physical activity. By way of another example, a bout of physical activity may begin when a heart rate monitor (e.g., worn by the user) is turned on and end when the heart rate monitor is turned off By way of another example, a bout of physical activity may begin when a heart rate monitor (e.g., worn by the user) is detected by an exercise machine (e.g., a treadmill or other exercise machine) such as via Bluetooth or ANT communications, and ends when the heart rate monitor is no longer detected by the machine. The exercise machine can communicate, for example, the beginning and ending of the bout of physical activity to the computing device 106.

The event detection module 1902 outputs an event indication 1922 to the prediction control module 1906 as well as to the glucose measurement prediction module 1908 for each identified bout of physical activity. Each event indication 1922 indicates a duration of time during which the bout of physical activity occurred. This time can be, for example, the beginning and ending times of the bout of physical activity.

The biological data detection module 1904 receives data stream 1920 and identifies glucose measurements in the data stream 1920. These identified glucose measurements are provided to the prediction control module 1906 as glucose measurements 1924. Additionally or alternatively, the biological data detection module 1904 detects any of a variety of other data included in the data stream 1920, such as heart rate data, HRV data, respiration rate data, and so forth that may affect glucose of the user 102 and provides that detected data to prediction control module 1906. Additionally or alternatively, the biological data detection module 1904 may detect other types of information from data stream 1920 (e.g. from a database on premises or in the cloud via network 112) based off what information the glucose prediction system 310 has about the user and cohorts (e.g., other users in the user population 108) having similar characteristics as the user to aid in predicting glucose measurements. For example, if biological data detection module 1904 has not detected information regarding the fitness level of the user (e.g., in situations in which the fitness level of the user is used by the glucose measurement prediction module 1908 in generating predicted glucose measurements), the biological data detection module 1904 detect or retrieve demographic information of the individual and estimate their fitness level based on the fitness level of their most similar cohort. The biological data detection module 1904 can provide any of this data or information to the glucose measurement prediction module 1908 for generation of the predicted glucose measurements.

The prediction control module 1906 identifies, for a bout of physical activity identified in an event indication 1922, an amount of time immediately preceding the bout of physical activity. This amount of time can be, for example, 30-40 minutes. The prediction control module 1906 identifies which glucose measurements 1924 correspond to the amount of time immediately preceding the bout of physical activity (e.g., have timestamps within the 30-40 minutes immediately preceding the bout of physical activity) and provides the glucose measurements to the glucose measurement prediction module 1908 as glucose measurements 1926.

The glucose measurement prediction module 1908 receives the event indication 1922 and the glucose measurements 1926 and predicts an impact of the physical activity bout on glucose of the user. The glucose measurement prediction module 1908 generates this prediction by generating, based on the glucose measurements 1926 (and optionally other physiological or demographic data), one or more predicted glucose measurements that the user would have had if, during the time the user was performing the bout of physical activity (as indicated by event indication 1922), the user had not performed the bout of physical activity. The predicted glucose measurements are output as predicted glucose measurements 1928 or provided to the feedback presentation system 122 as feedback indications 312.

In one or more implementations, the glucose measurement prediction module 1908 includes a machine learning system that generates the predicted glucose measurements. Machine learning systems refer to a computer representation that can be tuned (e.g., trained) based on inputs to approximate unknown functions. In particular, machine learning systems can include a system that utilizes algorithms to learn from, and make predictions on, known data by analyzing the known data to learn to generate outputs that reflect patterns and attributes of the known data. For instance, a machine learning system can include statistical time series forecasting models such as single order auto regressive models and second order auto regressive models, decision trees, support vector machines, linear regression, logistic regression, Bayesian networks, random forest learning, dimensionality reduction algorithms, boosting algorithms, artificial neural networks, deep learning, and so forth.

The machine learning system is trained, for example, by using training data that is sets of multiple glucose measurements for the user. These are, for example, sets of multiple consecutive glucose measurements over an amount of time (the same amount of time for which glucose measurements immediately preceding a bout of physical activity are identified by the prediction control module 1906, such as 30-40 minutes). The training data can be selected (e.g., randomly or pseudorandomly) based on glucose measurements received for a user over various days, weeks, months, and so forth. The training data includes glucose measurements for amounts of time that do not include bouts of physical activity. This allows the machine learning system to be trained to predict glucose measurements that occur in the absence of physical activity.

Known labels are associated with the sets of multiple data indicating what the subsequent glucose measurements were (e.g., the glucose measurements that occur immediately after those in the training data). The machine learning system is trained by updating weights or values of layers or coefficients in the machine learning system to minimize the loss between glucose measurements generated by the machine learning system for the training data and the corresponding known labels for the training data. Various different loss functions can be used in training the machine learning system, such as cross entropy loss, mean squared error loss, and so forth.

Additionally or alternatively, the machine learning system is trained to generate the predicted glucose measurements based on any of a variety of other data in the data stream 1920 or detected by the biological data detection module 1904. In such situations, the training data includes sets of the data for the user, such as sets of multiple data measured over an amount of time. For example, the machine learning system can be trained to generate the predicted glucose measurements based on any combination of physiological parameters (e.g., raw heart rate data, relative heart rate-based intensity measures, blood pressure measures, and so forth), demographic information (e.g., age, gender, and so forth), clinical information (medication stack data, prevalence of comorbidities data, fitness level data, etc.), and so forth.

The machine learning system is trained to generate multiple glucose measurements that occur after the training data (e.g., immediately after the training data).

The number of glucose measurements the machine learning system is trained to generate can be determined in a variety of different manners, such as determining an average duration for a bout of physical activity for the user based on previous bouts of physical activity for the user, receiving user input specifying the typical duration of a bout of physical activity for the user, and so forth. In one or more implementations, the machine learning system is trained to generate a number of glucose measurements following the training data that would typically be measured during a bout of physical activity (e.g., during the average duration or typical duration for a bout of physical activity). For example, assuming glucose measurements are obtained every 5 minutes and the typical duration of a bout of physical activity is 30 minutes, the machine learning system would be trained to generate a predicted glucose measurement after 5 minutes, after 10 minutes, after 15 minutes, after 20 minutes, after 25 minutes, and after 30 minutes. Additionally or alternatively, the machine learning system can be trained on data that is not in the immediate vicinity of the prediction time point (e.g., there could be a gap between the training period and the prediction time point).

Additionally or alternatively, the machine learning system is trained to generate a number of glucose measurements following the training data that would typically be measured during a bout of physical activity (e.g., during the average duration or typical duration for a bout of physical activity) and extending beyond the bout of physical activity by some duration of time (e.g., 15 or 20 minutes). For example, assume glucose measurements are obtained every 5 minutes and the typical duration of a bout of physical activity is 30 minutes, the machine learning system would be trained to generate a predicted glucose measurement after 5 minutes, after 10 minutes, after 15 minutes, after 20 minutes, after 25 minutes, after 30 minutes, after 35 minutes, after 40 minutes, and after 45 minutes.

In one or more implementations, the machine learning system generates a confidence level for each predicted glucose measurement. In such situations, the glucose prediction system 310 can take various actions based on the confidence levels for the predicted glucose measurements. For example, the glucose prediction system 310 may notify the user of predicted glucose measurements (as discussed in more detail below) only in situations where the confidence level of the predicted glucose measurements exceeds a threshold value (e.g., 75%). By way of another example, the glucose prediction system 310 may only notify the user of predicted glucose measurements for as long as the confidence level for the glucose measurements exceeds a threshold value (e.g., 75%)—after the confidence level no longer exceeds the threshold value the glucose prediction system 310 no longer notifies the user of the predicted glucose measurements regardless of whether the user is still engaged in a bout of physical activity.

In one or more implementations, the machine learning system generates a prediction interval for each predicted glucose measurements. For example, for the predicted glucose measurements after 10 minutes, a prediction interval or range is generated, such as a range of predicted glucose measurements having a confidence level that exceeds a threshold value (e.g., 75%). In such implementations, the glucose prediction system 310 may only notify the user of predicted glucose measurements if the actual glucose measurement of the user at the time of the bout of physical activity is outside of the prediction interval or range, or is beyond some threshold value (e.g., 250 mg/dL).

Accordingly, the user need not be notified of situations where there is not a meaningful difference between their actual glucose measurement and the predicted glucose measurement had they engaged in a bout of physical activity).

Additionally or alternatively, the glucose measurement prediction module 1908 can use any of various other models to generate the predicted glucose measurements 1928. For example, the glucose measurement prediction module 1908 can use physiological (pharmo-kinetics) or phenomenological models. E.g., glucose uptake can be modeled using ordinary differential equations that have parameters such as glucose uptake and exercise intensity.

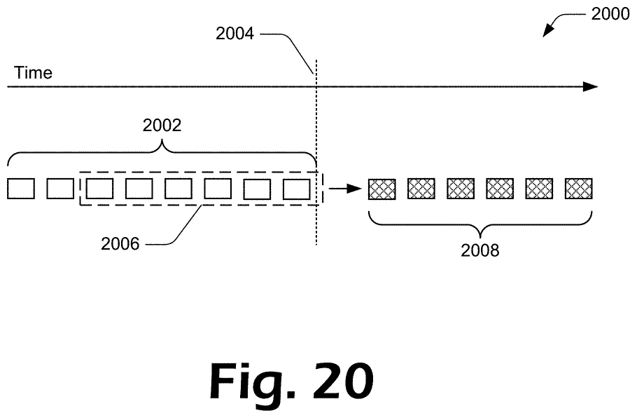

FIG. 20 illustrates an example 2000 of generating predicted glucose measurements. In the example 2000, multiple (eight) glucose measurements 2002 are illustrated (e.g., received as glucose measurements 1924). A time 2004 is illustrated that corresponds to the beginning of a bout of physical activity for the user (e.g., as indicated by the event indication 1922). The glucose measurements 2006 are a subset of the glucose measurements 2002 and are the glucose measurements immediately preceding the time 2004. The glucose measurements 2006 are used by the glucose measurement prediction module 1908 to generate predicted glucose measurements 2008 that occur immediately after the glucose measurements 2002. The predicted glucose measurements 2008 are generated for the duration of the bout of physical activity that began at the time 2004. Additionally or alternatively, the predicted glucose measurements 2008 may be generated for other durations of time, such as an amount of time (e.g., 15 or 20 minutes) extending beyond the bout of physical activity. This allows more meaningful glycemic impact feedback to be provided to the user 102. For example, the bout of physical activity is only 8 minutes in duration, extending the predicted glucose measurements 2008 by 15 or 20 minutes allows more accurate feedback to be provided to the user accounting for the time the user's body takes to react to the physical activity and make a meaningful change in the user's glucose measurements.

By way of another example, the predicted glucose measurements 2008 may be generated for different durations of time based on the intensity of the physical activity. For example, the higher the intensity of the physical activity, the longer the duration of time that the predicted glucose measurements 2008 are generated for.

Returning to FIG. 19, the training data used to train the machine learning system includes glucose measurements for the particular user 102. Accordingly, the machine learning system of the glucose measurement prediction module 1908 is trained or customized to the individual user 102, accounting for that individual user's body and glucose.

Although customized to the individual user 102, the machine learning system of the glucose measurement prediction module 1908 can optionally be re-trained over time in response to various events that may alter glucose management for the user. For example, the machine learning system can be re-trained using new training data after some period of time (e.g., 6 months or 1 year) to account for changes in the user's body. By way of another example, the machine learning system can be retrained using new training data obtained after a change in medication for the user.

The UI module 1910 optionally receives the predicted glucose measurements 1928 and causes the predicted glucose measurements 1928 to be displayed or otherwise presented (e.g., at computing device 106). This display or other presentation can take various forms, such as a static text display, graphic or video display, audio presentation, combinations thereof, and so forth. Additionally or alternatively the predicted glucose measurements 1928 are communicated to another user or system, such as to a health care provider or clinician. The glucose measurement prediction module 1908 optionally incorporates the predicted glucose measurements 1928 into a message or feedback to the user, such as a congratulatory message identifying the improvement in glucose (as indicated by the predicted glucose measurements 1928) over what the user's glucose measurements would have been without the bout of physical activity.

The UI module 1910 (or the feedback presentation system 122) can display or otherwise present predicted glucose measurements 1928 at any of a variety of times. In one or more implementations, the UI module 1910 (or the feedback presentation system 122) displays or otherwise presents predicted glucose measurements 1928 at the ending of a bout of physical activity. Additionally or alternatively, the UI module 1910 (or the feedback presentation system 122) displays or otherwise presents predicted glucose measurements 1928 at other times, such as in response to a user request for the predicted glucose measurements 1928, at particular time intervals (e.g., every evening or every morning), in response to a positive and meaningful change in glucose levels or dynamics (e.g., the user's glucose level dropped below a threshold amount or dropped by a threshold amount), and so forth.

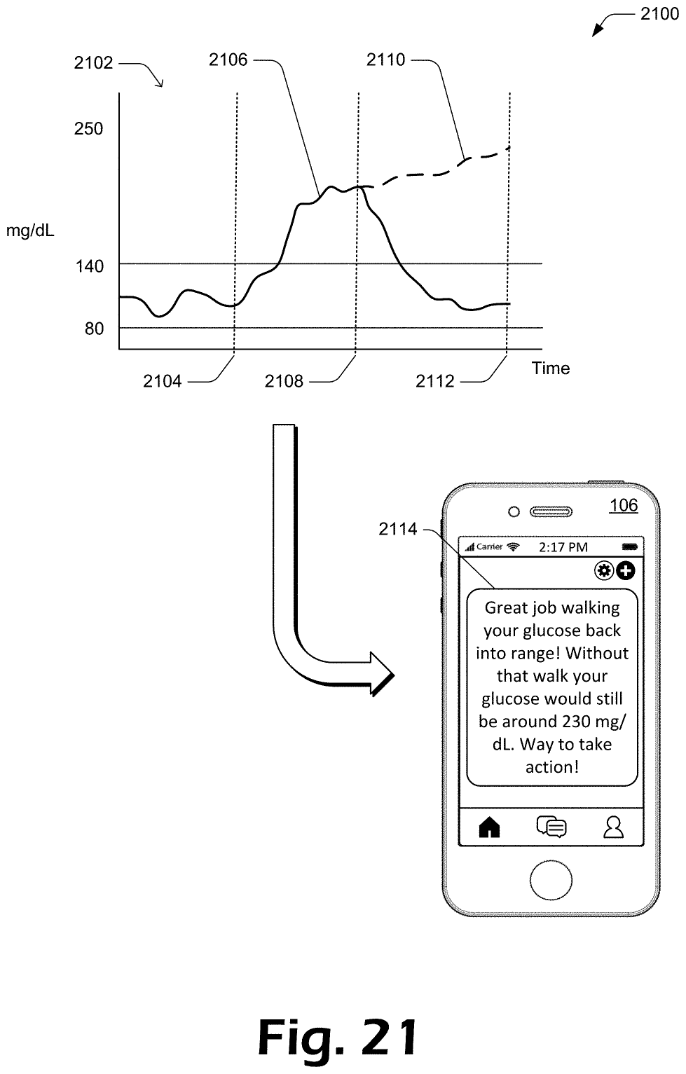

FIG. 21 illustrates an example 2100 of providing predicted glucose measurements. The example 2100 includes a graph 2102 plotting glucose measurements in mg/dL along the vertical axis against time along the horizontal axis. In the example 2100, assume that the user eats a meal at time 2104. The user's glucose measurements illustrated by the solid line 2106 increase after eating the meal. Further assume that the user begins a bout of physical activity at time 2108. As a result of the physical activity, the user's glucose measurements begin decreasing as shown. The glucose prediction system 310 generates predicted glucose measurements illustrated by the dashed line 2110 beginning at time 2108 (the beginning of the bout of physical activity) through time 2112 (e.g., the ending of the bout of physical activity). The glucose prediction system 310 provides feedback 2114 for display on the computing device 106 providing an indication of the predicted glucose measurements and the actual glucose measurements for the user. As illustrated, the feedback 2114 indicates the result of the bout of physical activity on the user's glucose and indicates how much better the user's glucose is than if he had not performed the bout of physical activity.

Returning to FIG. 19, the glucose measurement prediction module 1908 is discussed as providing the predicted glucose measurements 1928 to the UI module 1910 (or the feedback presentation system 122). The glucose prediction system 310 optionally takes additional actions based on the predicted glucose measurements 1928. In one or more implementations, these actions include notifying the glucose monitoring application 116 or the wearable glucose monitoring device 104 that the frequency with which glucose measurements 114 are produced can be reduced. For example, if the glucose prediction system 310 identifies a bout of physical activity, and the predicted glucose measurements 1928 for previous bouts of physical activity indicate an improvement in glucose measurements over the user 102 not engaging in the bout of physical activity, the glucose prediction system 310 can notify the glucose monitoring application 116 or wearable glucose monitoring device 104 that the frequency with which glucose measurements 114 are produced during a bout of physical activity can be reduced (e.g., from every 5 minutes to every 10 minutes), reducing the power expended to produce glucose measurements 114.

The discussions of the glucose prediction system 310 also include generating predicted glucose measurements 1928 in response to detecting bouts of physical activity. Additionally or alternatively, the glucose prediction system 310 generates predicted glucose measurements 1928 based on bouts of physical activity relative to other events, conditions, biological data, and so forth (e.g., based on any data in the data stream 1920). For example, the glucose prediction system 310 may generate predicted glucose measurements 1928 in response to physical activity occurring within a threshold amount of time (e.g., 30 minutes) of the user eating or drinking.

Furthermore, the discussions of the glucose prediction system 310 include predicting glucose measurements during bouts of physical activity. In one or more implementations, the glucose prediction system 310 differentiates between or among multiple different types of physical activity. For example, the event detection module 1902 may detect different types of physical activity, such as slow walking (e.g., at 60-79 steps per minute), medium walking (at 80-99 steps per minute), brisk walking (e.g., at 100-119 steps per minute), resistance training, and so forth. The glucose measurement prediction module 1908 can include a machine learning system trained using training data obtained during one of these types of physical activity, and can predict glucose measurements for the user for one of type of physical activity when a bout of another type of physical activity was performed by the user. For example, a machine learning system may be trained using training data obtained during bouts of slow walking. Subsequently, when the user engages in a bout of brisk walking, the glucose measurement prediction module 1908 can generate predicted glucose measurements 1928 indicating glucose if the user had instead engaged in a bout of slow walking. These predicted glucose measurements 1928 can be displayed or otherwise provided to the user, notifying the user of the improved glucose measurements resulting from brisk walking over slow walking.

Additionally or alternatively, in one or more implementations the glucose prediction system 310 predicts glucose measurements during times when the user is not engaged in a bout of physical activity. Such predicted glucose measurements can be generated analogous to the discussion herein regarding predicting glucose measurements during bouts of physical activity, except that the glucose measurement prediction module 1908 includes a machine learning system trained to generate predicted glucose measurements during a bout of physical activity. This allows the glucose prediction system 310 to provide feedback to the user or other person or system indicating what the user's predicted glucose measurements would be if the user had in fact engaged in a bout of physical activity.

Additionally or alternatively, the glucose prediction system 310 can predict glucose measurements based on any data included in the data stream 1920, such as data that indicates events or conditions that may affect glucose of the user 102. Such predicted glucose measurements can be generated analogous to the discussion herein regarding predicting glucose measurements during bouts of physical activity. This allows the glucose prediction system 310 to predict glucose measurements for other bouts or durations of time during which other activities or biological reactions are occurring.

By way of example, the data stream 1920 may include meal data. Accordingly, the glucose measurement prediction module 1908 can include a machine learning system trained using training data obtained over amounts of time when the user was not eating or drinking (and optionally what type of food or drink was being consumed by the user). This allows the glucose measurement prediction module 1908 to predict, for a duration of time during or after eating or drinking, glucose measurements for the user if the user had not consumed any food or drink (or had consumed a different type of food or drink). The differences between the actual glucose measurements and the predicted glucose measurements for the user if the user had not consumed any food or drink (or had consumed a different type of food or drink) can be displayed or otherwise provided to the user or other person or system.

By way of another example, the data stream 1920 may include sleep data. Accordingly, the glucose measurement prediction module 1908 can include a machine learning system trained using training data obtained over amounts of time when the user was not sleeping (or was in a particular sleep state). This allows the glucose measurement prediction module 1908 to predict, for a duration of time during or after sleeping, glucose measurements for the user if the user had not slept (or had slept in a different sleep state or for a different duration of time). The differences between the actual glucose measurements and the predicted glucose measurements for the user if the user had not slept (or had slept in a different sleep state or for a different duration of time) can be displayed or otherwise provided to the user or other person or system.

By way of another example, the data stream 1920 may include medication data. Accordingly, the glucose measurement prediction module 1908 can include a machine learning system trained using training data obtained over amounts of time when the user did take medication (and optionally what type or dose of medication was taken by the user). This allows the glucose measurement prediction module 1908 to predict, for a duration of time during or after taking medication, glucose measurements for the user if the user had not taken the medication (or had taken a different type or dose of medication). The differences between the actual glucose measurements and the predicted glucose measurements for the user if the user had not taken the medication (or had taken a different type or dose of medication) can be displayed or otherwise provided to the user or other person or system.

By way of another example, the data stream 1920 may include data that reflects stress management. Accordingly, the glucose measurement prediction module 1908 can include a machine learning system trained using training data obtained over amounts of time when the user was not stressed (or highly stressed). The user being stressed or highly stressed can be determined in various manners, such as various biological data (e.g., HRV, skin conductivity and temperature, respiration rate, EEG data, cortisol in biofluids, VOCs emitted from the skin) exceeding one or more threshold values, received user feedback on how stressed they are (e.g., via the glucose monitoring application 116 or other mobile application or desktop user interface), such as a rating on a 1-10 stress scale, and so forth. This allows the glucose measurement prediction module 1908 to predict, for a duration of time when the user is stressed (or highly stressed), glucose measurements for the user if the user were not stressed (or was not highly stressed). The differences between the actual glucose measurements and the predicted glucose measurements for the user if the user were not stressed (or not highly stressed) can be displayed or otherwise provided to the user or other person or system.

By way of another example, the data stream 1920 may include data regarding user engagement with glucose monitoring application 116. Accordingly, the glucose measurement prediction module 1908 can include a machine learning system trained using training data obtained over amounts of time when the user was not engaged with the glucose monitoring application 116 or was engaged with the glucose monitoring application 116 in a particular manner (e.g., what screens were viewed or what data was input). This allows the glucose measurement prediction module 1908 to predict, for a duration of time during or after engaging with the glucose monitoring application 116 (or engaging with the glucose monitoring application 116 in a particular manner), glucose measurements for the user if the user had not engaged with the glucose monitoring application 116 (or had engaged with the glucose monitoring application 116 in a different manner). The differences between the actual glucose measurements and the predicted glucose measurements for the user if the user had not engaged with the glucose monitoring application 116 (or had engaged with the glucose monitoring application 116 in a different manner) can be displayed or otherwise provided to the user or other person or system.

By way of another example, the data stream 1920 may include user interaction data that relates to user interactions with the computing device 106, with display of the computing device 106, or with other system components that indicate level of engagement with diabetes management. Accordingly, the glucose measurement prediction module 1908 can include a machine learning system trained using training data obtained over amounts of time when the user was interacting with the computing device 106, the display, or other system components (or optionally of what type of interaction the user had). This allows the glucose measurement prediction module 1908 to predict, for a duration of time during or after interacting with the computing device 106, the display, or other system components, glucose measurements for the user if the user had interacted with the computing device 106, the display, or other system (or had interacted with a different one of the computing device 106, the display, or other system). The differences between the actual glucose measurements and the predicted glucose measurements for the user if the user had interacted with the computing device 106, the display, or other system (or had interacted with a different one of the computing device 106, the display, or other system) can be displayed or otherwise provided to the user or other person or system.

By way of another example, the data stream 1920 may include data regarding user engagement with others of user population 108. Accordingly, the glucose measurement prediction module 1908 can include a machine learning system trained using training data obtained over amounts of time when the user was not engaged with other users of user population 108 (or optionally which other users of the user population 108 the user was engaged with). This allows the glucose measurement prediction module 1908 to predict, for a duration of time during or after engaging with other users of user population 108, glucose measurements for the user if the user had not engaged with other users of user population 108 (or had engaged with different users of the user population 108). The differences between the actual glucose measurements and the predicted glucose measurements for the user if the user had not engaged with other users of user population 108 (or had engaged with different users of the user population 108) can be displayed or otherwise provided to the user or other person or system.

Various different machine learning systems are discussed herein (e.g., for different types of data, different types of physical activity, and so forth). It should be noted that the glucose prediction system 310 can include a single one of these machine learning systems or any combination of the machine learning systems discussed herein. Accordingly, any of the predicted glucose measurements discussed herein can be generated concurrently with any other of the predicted glucose measurements.

The glucose measurement prediction module 1908 is discussed as including a machine learning system trained based on glucose measurements of the particular user 102. Additionally or alternatively, users are separated into different populations that have one or more similar characteristics. The user 102 is part of one of these different populations and the machine learning systems of the glucose measurement prediction module 1908 are trained using training data obtained from other users that are in the same population as the user 102 (e.g., and excluding any data obtained from users that are not in the same population as the user 102).

The populations can be defined in any of a variety of different manners. In one or more embodiments, the populations are defined by diabetes diagnosis (e.g., the user does not have diabetes, the user has Type 1 diabetes, or the user has Type 2 non-insulin-dependent diabetes). Additionally or alternatively, the populations are defined in different manners, for example age-based populations. E.g., populations are based on whether the user is an adult or a child (e.g., older than 18 or younger than 18), based on an age bracket the user is in (e.g., 0-5 years old, 5-10 years old, 10-20 years old, 20-30 years old, etc.), and so forth. By way of another example, populations can be defined based on additional medical conditions a user may have, such as hypertension, obesity, cardiovascular disease, neuropathy, nephropathy, retinopathy, Alzheimer's, depression, and so forth. By way of another example, populations can be defined based on user habits or activities, such as exercise or other physical activities, sleep patterns, time spent working versus at leisure, and so forth. By way of another example, populations can be defined based on the manner in which glucose measurements 114 are obtained or the equipment used to obtain glucose measurements 114, such as whether glucose measurements 114 are obtained via CGM, a brand of wearable glucose monitoring device 104, a frequency with which glucose measurements 114 are obtained, and so forth.

By way of another example, populations can be defined based on past glucose measurements 114 for users, such as by grouping users by clustering based on past glucose measurements 114. Examples of such clusters include users with high glycemic variability, users with frequent hypoglycemia, users with frequent hyperglycemia, and so forth. By way of another example, users can be grouped by clustering by using the past activity data of the users (e.g., step counts, energy expenditure, exercise minutes, sleep hours, and so forth obtained from activity trackers worn by the users). Examples of such clusters include users with high average steps per day, users with low average energy expenditure per day, users with low average number of sleep hours, and so forth.

It should be noted that, as discussed above, the predicted glucose measurements 1928 can be provided to the feedback presentation system 122 as feedback indications 312. In such situations the glucose prediction system 310 need not include the UI module 1910. Additionally or alternatively, the glucose measurements 1926 and the event indication 1922 can be provided to the feedback presentation system 122 as feedback indications 312. In such situations the feedback presentation system 122 identifies feedback to be provided to the user (or others, such as a clinician or pharmacist), as discussed in more detail below. The feedback presentation system 122 optionally identifies feedback to be provided to the user using any one or more of the techniques discussed herein with respect to the reportable diabetes management feedback identification module 408.

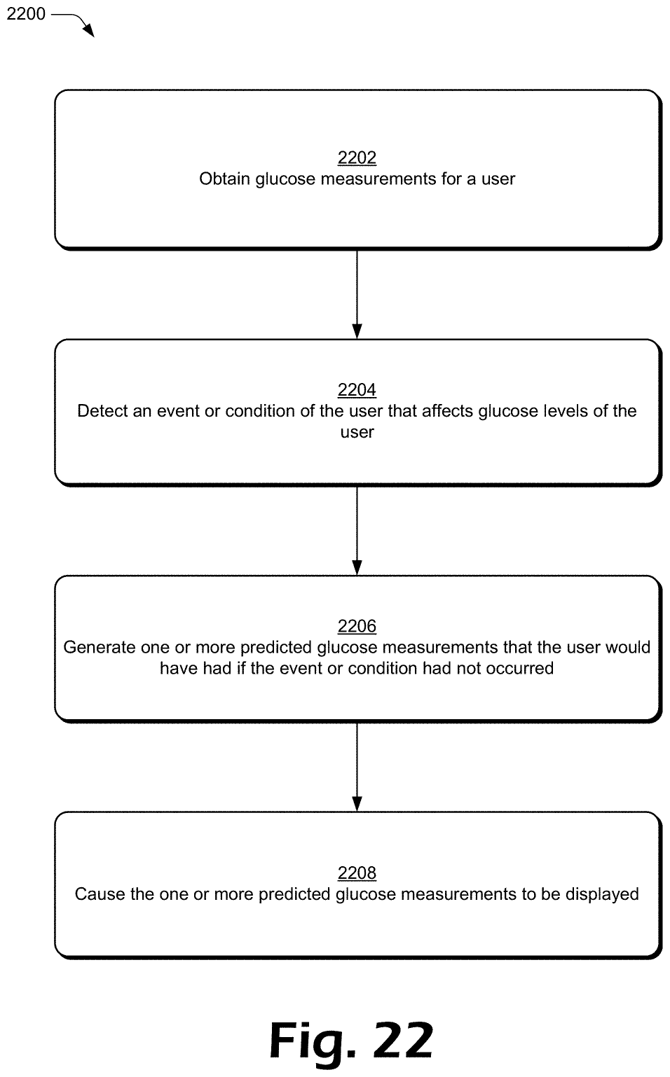

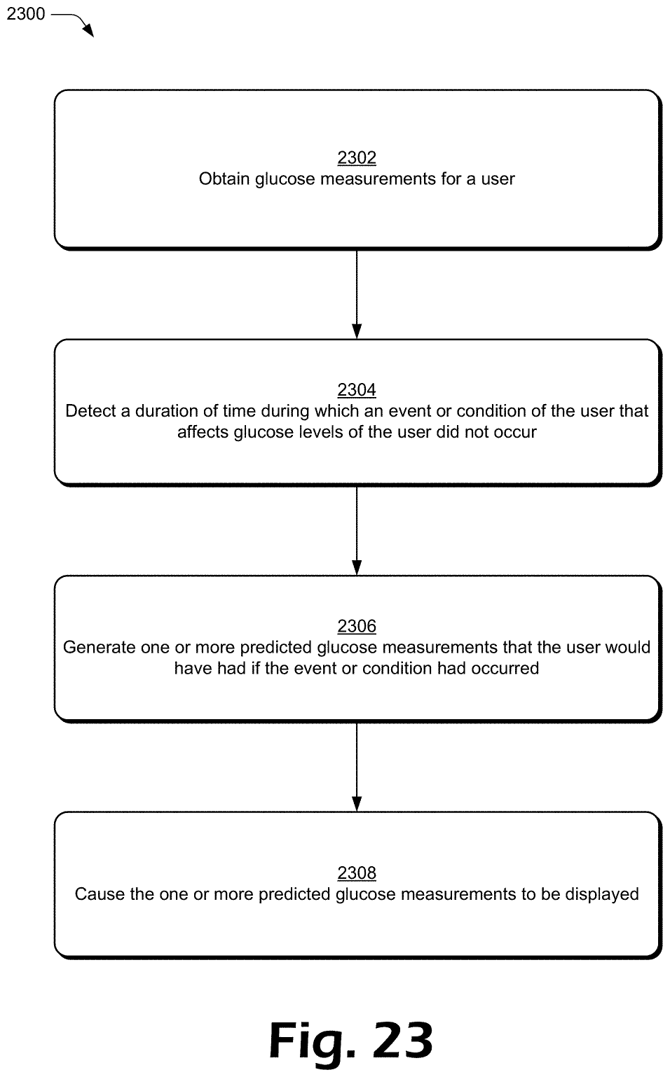

FIG. 22 and FIG. 23 describe examples of procedures for implementing glycemic impact prediction for improving diabetes management. Aspects of the procedures may be implemented in hardware, firmware, or software, or a combination thereof The procedures are shown as a set of blocks that specify operations performed by one or more devices and are not necessarily limited to the orders shown for performing the operations by the respective blocks.

FIG. 22 depicts a procedure 2200 in an example of implementing glycemic impact prediction for improving diabetes management. Procedure 2200 is performed, for example, by a diabetes management feedback generation system, such as the glucose prediction system 310 and optionally in part by a feedback presentation system, such as the feedback presentation system 122.

Glucose measurements for a user are obtained (block 2202). These glucose measurements are obtained from a glucose sensor of, for example, a continuous glucose level monitoring system with the glucose sensor being inserted at an insertion site of the user.

An event or condition of the user that affects glucose levels of the user is detected (block 2204). Any of a variety of events or conditions can be detected, such as bouts of physical activity, meals consumed, sleep, and so forth.

One or more predicted glucose measurements are generated (block 2206). The one or more predicted glucose measurements are glucose measurements that the user would have had if the event or condition had not occurred. These predicted glucose measurements are a prediction of the impact of the event or condition on glucose of the user.

The predicted glucose measurements are caused to be displayed (block 2208) or otherwise presented. Additionally or alternatively, the predicted glucose measurements can be communicated to or otherwise presented to a clinician, pharmacist, or other health care provider.

FIG. 23 depicts a procedure 2300 in an example of implementing glycemic impact prediction for improving diabetes management. Procedure 2200 is performed, for example, by a diabetes management feedback generation system, such as the glucose prediction system 310 and optionally in part by a feedback presentation system, such as the feedback presentation system 122.

Glucose measurements for a user are obtained (block 2302). These glucose measurements are obtained from a glucose sensor of, for example, a continuous glucose level monitoring system with the glucose sensor being inserted at an insertion site of the user.

A duration of time during which an event or condition of the user that affects glucose levels of the user did not occur is detected (block 2304). These events or conditions can be any of a variety of events or conditions, such as bouts of physical activity, meals consumed, sleep, and so forth.

One or more predicted glucose measurements are generated (block 2306). The one or more predicted glucose measurements are glucose measurements that the user would have had if the event or condition had occurred. These predicted glucose measurements are a prediction of the impact of the event or condition on glucose of the user.

The predicted glucose measurements are caused to be displayed (block 2308) or otherwise presented. Additionally or alternatively, the predicted glucose measurements can be communicated to or otherwise presented to a clinician, pharmacist, or other health care provider.

### Feedback Presentation System Architecture

Returning to FIG. 3, the feedback presentation system 122 receives the feedback indications 312 generated by the systems 304 — 310. Generally, the feedback presentation system 122 causes output of one or more user interfaces that present the diabetes management feedback indicated by the feedback indications 312. The feedback ranking module 320 ranks the various feedback indicated by the feedback indications 312 and provides the ranked feedback 332 to the feedback selection module 322. The feedback selection module 322 selects one or more of the ranked feedback 332 and provides the selected feedback 334 to the UI module 324. The UI module 324 receives the selected feedback 334 and causes the selected feedback 334 to be displayed or otherwise presented (e.g., at computing device 106). The feedback presentation system 122 also includes a feedback log 326 that is a record of feedback selected by the feedback selection module 322 and when (e.g., a date and time) the feedback was selected by the feedback presentation system 122 or displayed (or otherwise presented) by the UI module 324. The feedback selection module 322 can use this record in the feedback log 326 in selecting feedback as discussed in more detail below.

In one or more embodiments, the feedback presentation system 122 receives feedback indications 312 from the diabetes management feedback generation system 304. The feedback indications 312 from the diabetes management feedback generation system 304 include an indication of feedback corresponding to each rule (e.g., each rule 432 of FIG. 4) that was satisfied as discussed above. Additional information corresponding to the feedback is optionally included in the feedback indications 312, such as an indication of the rule that was satisfied, the feature to which the rule that was satisfied is directed, the time period to which the rule that was satisfied is directed, a magnitude of the improvement (e.g., the effect size), a type of feedback (e.g., improvement in glucose measurements for a given time period over one or more previous days, a time period of the day during which glucose measurements were the best (e.g., within an optimal range or closest to an optimal value), sustained positive patterns), and so forth.

Additionally or alternatively, the feedback presentation system 122 receives feedback indications 312 from the glucose level deviation detection system 306. The feedback indications 312 from the glucose level deviation detection system 306 include a deviation identification of each deviation detected by the glucose level deviation detection system 306 (e.g., a deviation identification 1030 corresponding to each deviation indication 1026 and deviation indication 1028). Additional information corresponding to the deviation identification is optionally included in the feedback indications 312, such as an indication of the significance of the deviation (e.g., a magnitude or size of the deviation, a direction of the deviation (e.g., positive or negative impact on diabetes management)), an indication of whether the deviation identification is a positive acknowledgement or a preemptive warning, and so forth.

Additionally or alternatively, the feedback presentation system 122 receives feedback indications 312 from the behavior modification identification system 308. The feedback indications 312 from the behavior modification identification system 308 include the behavior modification feedback that is mapped to by at least one topic (e.g., all of the behavior modification feedback that is mapped to by at least one topic 1532), such as behavior modification feedback (actionable goals).

Additionally or alternatively, the feedback presentation system 122 receives feedback indications 312 from the glucose prediction system 310. The feedback indications 312 from the glucose prediction system 310 includes as feedback the predicted glucose measurements (e.g., the predicted glucose measurements 1928) and contextual information for the predicted glucose measurements (e.g., an indication that the predicted glucose measurements are the result of physical activity of the user, such as walking).

The feedback indications 312 include various different diabetes management feedbacks that are generated by the feedback generation system 120. The feedback ranking module 320 receives the feedback indications 312 and ranks the various feedback indicated by the feedback indications 312, providing the ranked feedback 332 to the feedback selection module 322. The feedback ranking module 320 ranks or prioritizes the indicated feedbacks, and the feedback selection module 322 selects feedback for presentation to the user. The feedback presentation system 122 can rank or prioritize feedback for selection in a variety of different manners.

In one or more implementations, different features can be directed to different rules, and feedback corresponding to a satisfied rule can be prioritized or ranked based on the type of feature to which the rule that was satisfied is directed. For example, feedback resulting from features that are directed to a clinical guidelines type can be ranked higher than features that are directed to a recent glucose measurements type, features that are directed to a recent glucose measurements type can be ranked higher than features that typically have a large amount of variability, and so forth. The type of a feature can be determined in various manners, such as specified by a developer or designer of the feedback presentation system 122, specified by a health care provider or professional, and so forth. E.g., this allows feedback corresponding to higher priority or ranked features to be selected for display or other presentation to the user over other lower priority or ranked features.

Additionally or alternatively, the top option or highest ranked feedback for each type of features is chosen. The different types of features are ranked in any of various manners, such as specified by a developer or designer of the feedback presentation system 122, specified by a health care provider or professional, and so forth. This allows the highest ranked feedback for each type of features to be identified and then allows those feedbacks to be ranked against each other based on the type of feature.

Additionally or alternatively, different feedback can have different safety ratings, such as a Boolean value (e.g., 0 corresponding to a low or non-urgent safety rating, 1 corresponding to a high or urgent safety rating). E.g., this allows feedback corresponding to urgent or safety-related features (e.g., not staying within ranges or exceeding threshold glucose levels) to be selected over other non-urgent or non-safety-related features and display or otherwise present more critical diabetes management feedback to the user. The safety rating of feedback can be determined in various manners, such as specified by a developer or designer of the feedback presentation system 122, specified by a health care provider or professional, and so forth.

Additionally or alternatively, different feedback can correspond to different rules that involve different values that are satisfied (e.g., threshold values, amounts of time in range, and so forth). The different feedback can be prioritized or ranked based on the size or amount of these different values. For example, feedback corresponding to rules that are satisfied by a larger amount (e.g., a larger amount above or below a threshold value, a larger amount of time in range, and so forth) can be ranked higher than rules that are satisfied by a smaller amount.

Additionally or alternatively, the different feedback can be prioritized or ranked based on how recently the corresponding rule was satisfied (e.g., as indicated by the feedback log 326). For example, feedback corresponding to a rule that was satisfied less recently is ranked higher than feedback corresponding to a rule that was satisfied more recently. E.g., this allows different feedback (corresponding to the different rules) to be provided to the user and avoids repeating feedback too frequently.

Additionally or alternatively, the different feedback can be prioritized or ranked based on how frequently the corresponding rule is satisfied. For example, feedback corresponding to a rule that is satisfied less frequently is ranked higher than feedback corresponding to a rule that is satisfied more frequently. E.g., this allows different feedback (corresponding to the different rules) to be provided to the user and avoids repeating feedback too frequently.

Additionally or alternatively, different detected patterns can have different sizes and these detected patterns can be mapped to different topics as discussed above. The different feedback can be prioritized or ranked based on, for the feedback mapped to by a topic, the size of the detected pattern mapped to that topic. For example, feedback mapped to by a topic to which a pattern having a larger size is mapped can be ranked higher than feedback mapped to by a topic to which a pattern having a smaller size is mapped.

Additionally or alternatively, the different feedback can be prioritized or ranked based on how recently (e.g., as indicated by the feedback log 326) a detected pattern was mapped to a topic. For example, feedback corresponding to a topic to which a detected pattern was less recently mapped is ranked higher than feedback corresponding to a topic to which a detected pattern was more recently mapped. E.g., this allows different feedback (corresponding to the different detected patterns) to be provided to the user and avoids repeating feedback too frequently.

Additionally or alternatively, the different feedback can be prioritized or ranked based on how frequently a detected pattern was mapped to a topic. For example, feedback corresponding to a topic to which detected patterns are less frequently mapped is ranked higher than feedback corresponding to a topic to which detected patterns are more frequently mapped. E.g., this allows different feedback (corresponding to the different detected patterns) to be provided to the user and avoids repeating feedback too frequently.

Additionally or alternatively, the different feedback can be prioritized or ranked based on a tone or type of communication. For example, feedback may be of an informational tone type (e.g., providing educational information to the user) or a constructive tone type (e.g., suggestions on behavioral changes the user can make). Feedback of a constructive tone type can be ranked higher than feedback of an informational tone type. E.g., this allows feedback that is more likely to result in the user making changes that improves his diabetes management being provided to the user rather than information that is merely educational. The tone or type of feedback can be determined in various manners, such as specified by a developer or designer of the feedback presentation system 122, specified by a health care provider or professional, and so forth.

The feedback ranking module 320 employs any of a variety of different rules or criteria to rank the feedback indicated by feedback indications 312. In one or more embodiments, the feedback ranking module 320 ranks feedback received from the diabetes management feedback generation system 304 based on the type of feedback (e.g., improvement in glucose measurements for a given time period over one or more previous days, a time period of the day during which glucose measurements were the best (e.g., within an optimal range or closest to an optimal value), sustained positive patterns), and so forth.

For each of the feedback corresponding to improvement in glucose measurements for a given time period over one or more previous days type and the time period of the day during which glucose measurements were the best type, the feedback ranking module 320 ranks the feedback by magnitude, then by time period, then by corresponding feature (e.g., metric). When ranking by magnitude, feedback corresponding to a feature having a larger magnitude (larger improvement) is ranked higher than feedback corresponding to a feature having a smaller magnitude (smaller improvement). When ranking by time period, an overnight or sleep time period is ranked lower than other time periods (e.g., allowing feedback focused on time periods where a user can take action, such as after a meal, so that a user is more likely to take action). These other time periods have the same ranking relative to one another.

When ranking by corresponding feature, the ranking varies based on the time period. For example, for the overnight or sleep time period the rankings from highest to lowest are time in a narrow range (e.g., between 70 mg/dL and 140 mg/dL), then time in a wider range (e.g., between 70 mg/dL and 180 mg/dL), then time below a particular glucose level (e.g., 70 mg/dL), then time above a particular glucose level (e.g., 250 mg/dL), then mean glucose level. For other time periods the rankings from highest to lowest are maximum glucose level, then time in a narrow range (e.g., between 70 mg/dL and 140 mg/dL), then time in a wider range (e.g., between 70 mg/dL and 180 mg/dL), then time above a particular glucose level (e.g., 250 mg/dL), then time below a particular glucose level (e.g., 70 mg/dL), then mean glucose level.

In one or more implementations, when ranking by corresponding feature, the ranking can vary based on certain values for certain features. For example, if the time in a range (e.g., between 70 mg/dL and 180 mg/dL) is at least a threshold amount (e.g., 70% of the time period) then the time in a narrow range (e.g., between 70 mg/dL and 140 mg/dL) is ranked highest amongst the features and the mean glucose level is ranked second highest amongst the features. By way of another example, if the coefficient of variation is at least a threshold amount (e.g., 30% during the time period) then the time above a particular glucose level (e.g., 250 mg/dL) is ranked highest amongst the features and the maximum glucose level is ranked second highest amongst the features.

For the sustained positive patterns type, the feedback ranking module 320 ranks the feedback by magnitude then by corresponding feature (e.g., metric). When ranking by magnitude, feedback corresponding to a feature having a larger magnitude (longer streak duration) is ranked higher than feedback corresponding to a feature having a smaller magnitude (smaller streak duration). When ranking by corresponding feature, the ranking varies based on the time period (and is the same discussed above with reference to the feedback corresponding to improvement in glucose measurements for a given time period over one or more previous days type and the time period of the day during which glucose measurements were the best type).

In one or more embodiments, the feedback ranking module 320 ranks feedback received from the behavior modification identification system 308 by corresponding feature (e.g., metric), then by magnitude, then by time of day (e.g., time period). When ranking by time period, the time periods are ranked (from most important to least important) in the order: evening (e.g., after dinner), morning (e.g., after breakfast), midday (e.g., after lunch) and overnight (e.g., sleep).

In one or more implementations, when ranking by corresponding feature, the rankings in the various time frames is the same. For example, for the rankings from highest to lowest are maximum glucose level, then time above a particular glucose level (e.g., 250 mg/dL), then time in a range (e.g., between 70 mg/dL and 180 mg/dL), then coefficient of variation, then mean glucose. Additionally or alternatively, different rankings may be used for different time periods. For example, the maximum glucose metric may be de-prioritized (e.g., ranked lower) in the overnight time period since people tend to have less control over their maximum glucose during that time period so it is less actionable than other metrics.

In one or more implementations, when ranking by corresponding feature, the ranking can vary based on certain values for certain features. For example, if the time above a particular glucose level (e.g., 250 mg/dL) is at least a threshold amount (e.g., 1% of the time period), then for the overnight or sleep time period the time above a particular glucose level (e.g., 250 mg/dL) is ranked highest amongst the features and the overnight glucose level is ranked second highest amongst the features. By way of another example, if the coefficient of variation is at least a threshold amount (e.g., 30% during the time period) then the maximum glucose level is ranked highest amongst the features and the coefficient of variation is ranked second highest amongst the features.

When ranking by magnitude, the magnitude of detected patterns mapping to topics that map to the feedback are compared. For a detected pattern having a larger magnitude (larger size), the feedback that is mapped to the same topic as the detected pattern is ranked higher than feedback that is mapped to the same topic as a detected pattern having a smaller magnitude (smaller size).

In one or more implementations, the feedback ranking module 320 treats safety-related feedback separately from other feedback. Safety-related feedback refers to feedback indicating a serious health risk for the user and one for which the user should seek assistance from a medical professional quickly or take a remedial action quickly. For example, feedback resulting from the time below a particular glucose level (e.g., 70 mg/dL) being at least a threshold amount (e.g., 1% of the time period) or the time above a particular glucose level (e.g., 250 mg/dL) being at least a threshold amount (e.g., 1% of the time period) is considered safety-related feedback. The feedback ranking module 320 provides this safety-related feedback to the feedback selection module 322 as safety-related feedback 336, allowing the feedback selection module 322 to quickly provide the safety-related feedback 336 to the user.

The feedback as ranked by the feedback ranking module 320 is output as ranked feedback 332. The feedback selection module 322 selects one or more of the ranked feedback 332 and the safety-related feedback 336, and provides the selected feedback 334 to the UI module 324. In one or more implementations, the feedback selection module 322 provides, as the selected feedback 334, the safety-related feedback 336 and all of the ranked feedback 332 in its order of ranking (e.g., categorized based on which of the systems 304-310 the feedback was received from). For example, the selected feedback 334 may be the safety-related feedback 336, the feedback from the diabetes management feedback generation system 304 in the order ranked by feedback ranking module 320, followed by the feedback from behavior modification identification system 308 in the order ranked by the feedback ranking module 320. The selected feedback 334 may include only a subset of the ranked feedback 332, such as one or two highest-ranked feedback of the ranked feedback 332.

In one or more implementations, safety-related communications (e.g., the safety-related feedback 336) are output by the UI module 324 quickly, such as within 3 or 5 minutes of receipt by the UI module 324. Accordingly, the safety-related communications are provided to the user quickly, allowing him or her to take appropriate medical or remedial action.

In one or more implementations, multiple reports are generated by the UI module 324 at different intervals. For example, daily and weekly reports are generated that include any feedback from the diabetes management feedback generation system 304 (as ranked by the feedback ranking module 320), followed by any feedback from the behavior modification identification system 308 (as ranked by the feedback ranking module 320). Any safety-related feedback is optionally included in the reports as well.

By way of example, for feedback corresponding to sustained positive patterns received from diabetes management feedback generation system 304, the selected feedback 334 can include a longest streak (sustained positive pattern) for each time period (e.g., of a day) or a longest streak (sustained positive pattern) across all time periods. By way of another example, for feedback corresponding to a time period of the day during which glucose measurements were the best (e.g., a time period during which glucose measurements were within an optimal range or closest to an optimal value) received from the diabetes management feedback generation system 304, the selected feedback 334 can include feedback identifying the best time period of the day in a daily report and feedback identifying the best time period of each of three separate days in a weekly report. By way of another example, for feedback corresponding to improvement in glucose measurements for a given time period over one or more previous days received from the diabetes management feedback generation system 304, the selected feedback 334 can include the highest ranked feedback identifying the best time period of the day (e.g., the time period of the day when glucose measurements were within an optimal range or closest to an optimal value) in a daily report and feedback identifying the best time periods of each of three separate days (e.g., the time periods of each of three separate days when glucose measurements were within an optimal range or closest to an optimal value) in a weekly report.

In one or more implementations, the feedback presentation system 122 provides other feedback as it is generated or received by the feedback presentation system 122 (e.g., in real time). For example, feedback identifying a deviation detected by the glucose level deviation detection system 306 is provided to UI module 324 as selected feedback 334 in response to receipt of the corresponding feedback indication 312. By way of another example, feedback identifying a predicted glucose measurement generated by the glucose prediction system 310 is provided to UI module 324 as selected feedback 334 in response to receipt of the corresponding feedback indication 312.

In one or more embodiments, the feedback selection module 322 provides as selected feedback 334 various glucose reporting feedback, as part of a regular report (e.g., daily or weekly report) or at other times. Which glucose reporting feedback is included in selected feedback 334 can vary based on certain values for certain features. For example, if the time below a particular glucose level (e.g., 70 mg/dL) for a time period is at least a threshold amount (e.g., 1% of the time period), then the time below the particular glucose level is included in selected feedback 334. By way of another example, if the time above a particular glucose level (e.g., 250 mg/dL) for a time period is at least a threshold amount (e.g., 1% of the time period), then time above the particular glucose level is included in selected feedback 334. By way of another example, if the time in a range (e.g., between 70 mg/dL and 180 mg/dL) for a time period is less than a threshold amount (e.g., 70% of the time period) then the time in range is included in selected feedback 334. By way of another example, if the time in a range (e.g., between 70 mg/dL and 180 mg/dL) for a time period is not less than a threshold amount (e.g., 70% of the time period) then the time in range as well as the time in a narrower range (e.g., between 70 mg/dL and 130 mg/dL) are included in selected feedback 334. By way of another example, if the coefficient of variation is at least a threshold amount (e.g., 30% during a time period) then an indication of a coefficient of variation having high variability is included in the selected feedback 334. By way of another example, if the coefficient of variation is within a particular range (e.g., between 17% and 29% during a time period) then an indication of a coefficient of variation having low variability (stable glucose) is included in the selected feedback 334. By way of another example, if the coefficient of variation is less than a threshold amount (e.g., 17% during a time period) and the time in a range (e.g., between 70 mg/dL and 180 mg/dL) for the time period is at least a threshold amount (e.g., 70% of the time period) and the time above a particular glucose level (e.g., 250 mg/dL) for the time period is less than a threshold amount (e.g., 1% of the time period), then an indication of a coefficient of variation having low variability (stable glucose) is included in the selected feedback 334.

In one or more embodiments, the feedback presentation system 122 maintains a feedback log 326, which is a record of feedback selected by the feedback selection module 322 and when (e.g., a date and time) when the feedback was selected by the feedback presentation system 122 or displayed (or otherwise presented) by the UI module 324. The feedback log 326 can also include the rankings of the selected feedback 334 (and optionally all of the ranked feedback 332), allowing the feedback selection module 322 to factor in previous rankings of feedback (e.g., on previous days), such as to determine to not select feedback having been previously ranked low multiple times.

Using feedback log 326 allows different feedback to be provided to the user and avoids repeating feedback too frequently. For example, the feedback selection module 322 may determine to not include particular feedback in the selected feedback 334 for a daily report in response to the feedback log 326 indicating that the same feedback was included in selected feedback 334 for the previous day's daily report. By way of another example, the feedback selection module 322 may determine to not include particular feedback in the selected feedback 334 for a weekly report in response to the feedback log 326 indicating that the same feedback was included in selected feedback 334 for the daily reports for each of the previous two weeks.

In one or more embodiments, in situations in which ranked feedback 332 includes feedback corresponding to approximately the same time periods received from different ones of the systems 304, 306, 308, and 310, the feedback selection module 322 can select the feedback from only one of the systems 304, 306, 308, and 310, accounting for potentially contradictory feedback corresponding to approximately the same time periods from being displayed to the user. For example, the diabetes management feedback generation system 304 and the behavior modification identification system 308 each provide feedback corresponding to the same time period, the feedback from the diabetes management feedback generation system 304 would typically be more positive whereas the feedback from behavior modification identification system 308 would typically be more negative (e.g., indicating an action to take to improve diabetes management). In such situations, the feedback selection module 322 selects only one of the two feedbacks (e.g., selects the feedback from the behavior modification identification system 308). By way of another example, if hypoglycemia is detected within a time period (e.g., if the time below a particular glucose level (such as 70 mg/dL) is at least a threshold amount (such as 1% of the time period)), the feedback selection module 322 does not select feedback corresponding to mean glucose features for that time period received from the diabetes management feedback generation system 304. This prevents the feedback selection module 322 from selecting feedback received from the diabetes management feedback generation system 304 indicating that the mean glucose for the user during the time period was good (e.g., as the mean glucose was likely lower due to the hypoglycemia). Additionally or alternatively, the feedback selection module 322 can select the feedback from two or more of the systems 304, 306, 308, and 310 to allow multiple feedback for approximately the same time periods to be displayed to the user. In such situations, the feedback selection module 322 includes functionality to analyze the multiple feedback and mitigate (e.g., not select) uncanny or contradictory feedback from being displayed for approximately the same time period.

In one or more implementations, the UI module 324 receives the selected feedback 334 and causes the selected feedback 334 to be displayed or otherwise presented (e.g., at computing device 106). This display or other presentation can take various forms, such as a static text display, graphic or video display, audio presentation, combinations thereof, and so forth. Additionally or alternatively, the selected feedback 334 can be communicated to or otherwise delivered to others, such as a clinician (e.g., the user's primary care physician or nurse), a pharmacist, and so forth.

Various examples of the display of feedback are included herein. Such examples include feedback 504 of FIG. 5, feedback 604 of FIG. 6, feedback 704 of FIG. 7, behavior modification feedback 1612 of FIG. 16, feedback 2114 of FIG. 21, and so forth.

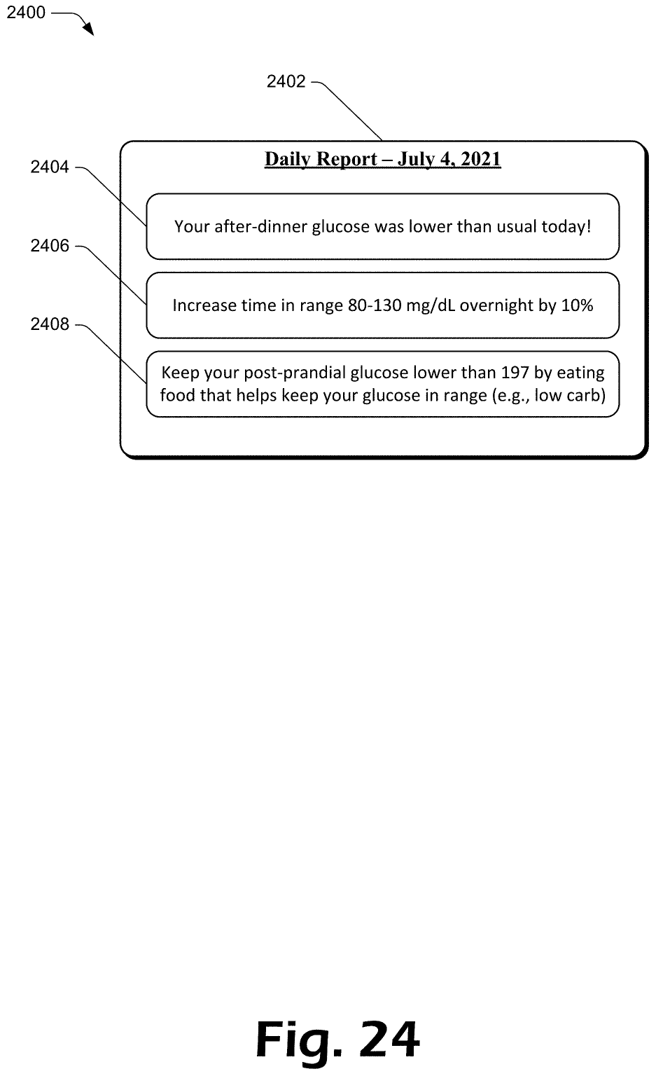

FIG. 24 illustrates an example 2400 of feedback. The example 2400 is for a daily report 2402, which can be displayed by the computing device 106, communicated to another user or device (e.g. via email), and so forth. The daily report 2402 includes feedback 2404 generated by the diabetes management feedback generation system 304 as well as feedback 2406 and 2408 generated by the behavior modification identification system 308.

Returning to FIG. 3, it should be noted that various techniques that can be used for determining which feedback to select for display or other presentation are discussed above with respect to the individual systems 304, 306, 308, and 310. These include selecting feedback corresponding to a rule that was satisfied by the diabetes management feedback generation system 304, selecting deviations by the glucose level deviation detection system 306, selecting a mapped to behavior modification by the behavior modification identification system 308, and so forth. Any of the techniques discussed above with respect to the systems 304, 306, 308, and 310 can be used by the feedback selection module 322 in selecting feedback.

It should also be noted that although various functionality is discussed herein as being performed by the feedback generation system 120 or the feedback presentation system 122, additionally or alternatively at least some of this functionality can be performed by the other of the systems 120 and 122. For example, each of the systems 304, 306, 308, and 310 may select a subset of feedback and provide that selected subset of feedback as the feedback indications 312, and the feedback selection module 322 in turn selects from those subsets of feedback. By way of another example, any of the functionality discussed above with reference to any of the systems 304, 306, 308, and 310 can additionally or alternatively be performed by the feedback presentation system 122.

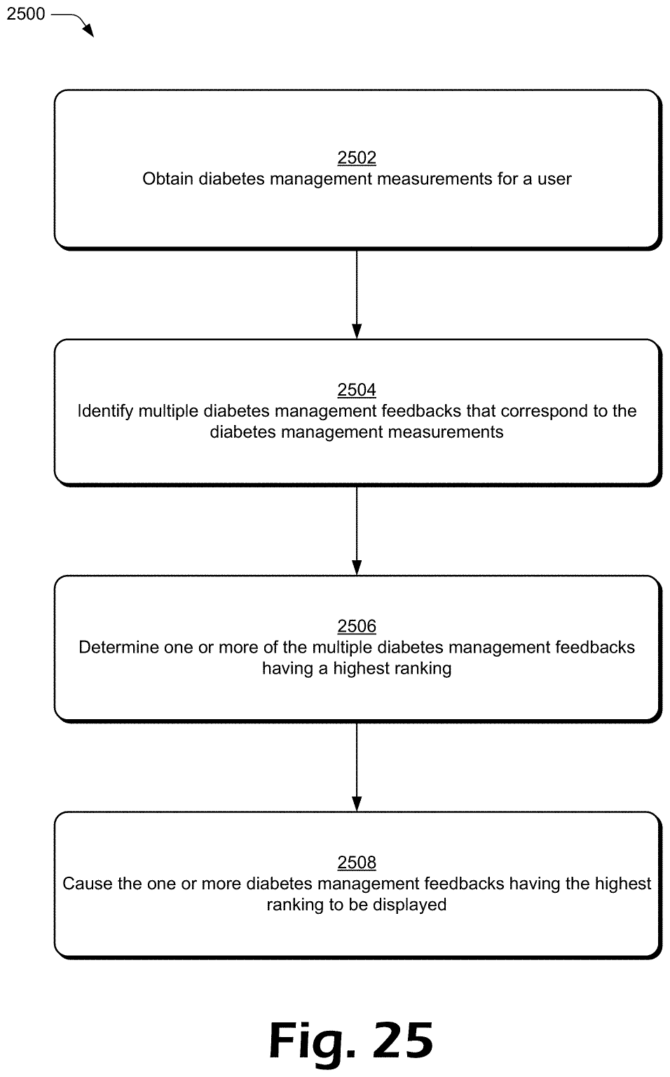

FIG. 25 describes an example of a procedure for implementing ranking feedback for improving diabetes management. Aspects of the procedures may be implemented in hardware, firmware, or software, or a combination thereof The procedure is shown as a set of blocks that specify operations performed by one or more devices and is not necessarily limited to the orders shown for performing the operations by the respective blocks. Procedure 2500 is performed, for example, by a diabetes management feedback generation system and a feedback presentation system, such as the feedback generation system 120 and the feedback presentation system 122.

Diabetes management measurements for a user are obtained (block 2502). These diabetes management measurements are, for example, glucose measurements are obtained from a glucose sensor of a continuous glucose level monitoring system with the glucose sensor being inserted at an insertion site of the user.

Multiple diabetes management feedbacks that correspond to the diabetes management measurements are identified (block 2504). These diabetes management feedbacks are generated by various systems, and can include, for example, feedback identifying improvements in glucose measurements for a given time period over one or more previous days, feedback identifying a time period of the day during which glucose measurements were the best (e.g., within an optimal range or closest to an optimal value), feedback identifying sustained positive patterns (e.g., good diabetes management for a same time period in each of multiple days), feedback identifying deviations in glucose measurements between time periods, feedback identifying potential behavior modification (e.g., actions) that a user could take to engage in beneficial diabetes management behavior, feedback identifying what a user's glucose would have been had the particular events or conditions not occurred or not been present (e.g., the user had not taken a walk), and so forth.

One or more of the multiple diabetes management feedbacks having a highest ranking is determined (block 2506). These rankings can be based on various rules or criteria, such as ranking diabetes management feedback based on the time period of the day corresponding to the diabetes management feedback, based on a feature corresponding to the diabetes management feedback, based on a magnitude of an improvement corresponding to the feedback, and so forth.

The one or more diabetes management feedbacks are caused to be displayed (block 2508) or otherwise presented. Additionally or alternatively, the predicted glucose measurements can be communicated to or otherwise presented to a clinician, pharmacist, or other health care provider.

### Example System and Device

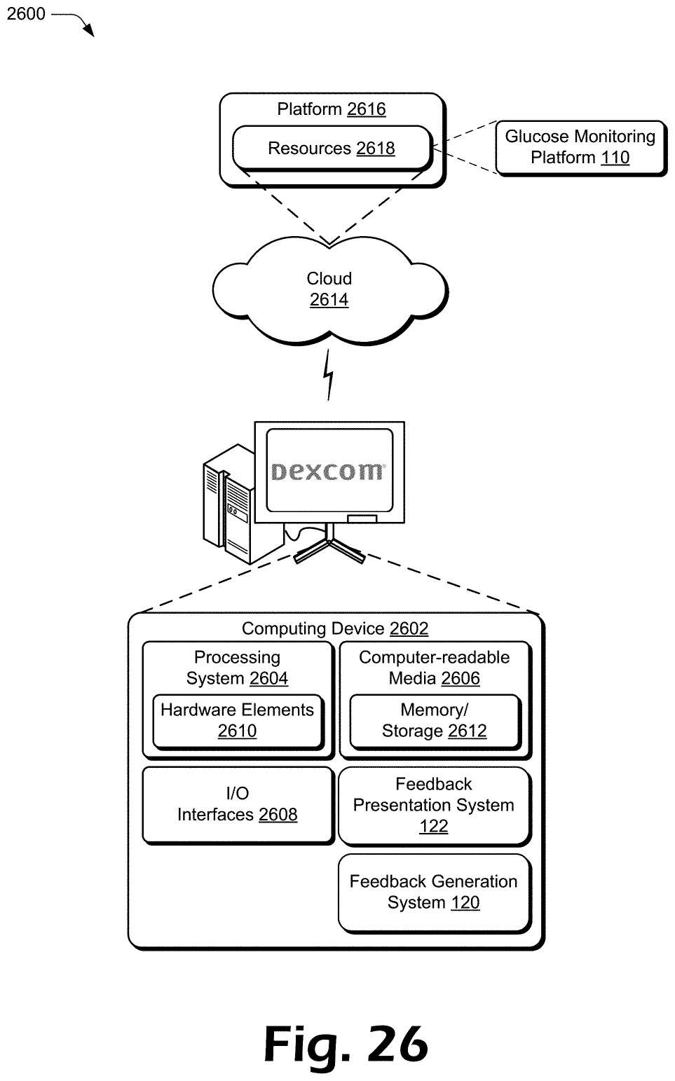

FIG. 26 illustrates an example of a system generally at 2600 that includes an example of a computing device 2602 that is representative of one or more computing systems and/or devices that may implement the various techniques described herein. This is illustrated through inclusion of the feedback generation system 120 and the feedback presentation system 122. The computing device 2602 may be, for example, a server of a service provider, a device associated with a client (e.g., a client device), an on-chip system, and/or any other suitable computing device or computing system.

The example computing device 2602 as illustrated includes a processing system 2604, one or more computer-readable media 2606, and one or more I/O interfaces 2608 that are communicatively coupled, one to another. Although not shown, the computing device 2602 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 2604 is representative of functionality to perform one or more operations using hardware. Accordingly, the processing system 2604 is illustrated as including hardware elements 2610 that may be configured as processors, functional blocks, and so forth. This may include implementation in hardware as an application specific integrated circuit or other logic device formed using one or more semiconductors. The hardware elements 2610 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically-executable instructions.

The computer-readable media 2606 is illustrated as including memory/storage 2612. The memory/storage 2612 represents memory/storage capacity associated with one or more computer-readable media. The memory/storage component 2612 may include volatile media (such as random access memory (RAM)) and/or nonvolatile media (such as read only memory (ROM), Flash memory, optical disks, magnetic disks, and so forth). The memory/storage component 2612 may include fixed media (e.g., RAM, ROM, a fixed hard drive, and so on) as well as removable media (e.g., Flash memory, a removable hard drive, an optical disc, and so forth). The computer-readable media 2606 may be configured in a variety of other ways as further described below.

Input/output interface(s) 2608 are representative of functionality to allow a user to enter commands and information to computing device 2602, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 2602 may be configured in a variety of ways as further described below to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof The features of the techniques described herein are platform-independent, meaning that the techniques may be implemented on a variety of commercial computing platforms having a variety of processors.

An implementation of the described modules and techniques may be stored on or transmitted across some form of computer-readable media. The computer-readable media may include a variety of media that may be accessed by the computing device 2602. By way of example, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, or article of manufacture suitable to store the desired information and which may be accessed by a computer.

“Computer-readable signal media” may refer to a signal-bearing medium that is configured to transmit instructions to the hardware of the computing device 2602, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, communication media include wired media such as a wired network or direct-wired connection, and wireless media such as acoustic, RF, infrared, and other wireless media.

As previously described, hardware elements 2610 and computer-readable media 2606 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 2610. The computing device 2602 may be configured to implement particular instructions and/or functions corresponding to the software and/or hardware modules. Accordingly, implementation of a module that is executable by the computing device 2602 as software may be achieved at least partially in hardware, e.g., through use of computer-readable storage media and/or hardware elements 2610 of the processing system 2604. The instructions and/or functions may be executable/operable by one or more articles of manufacture (for example, one or more computing devices 2602 and/or processing systems 2604) to implement techniques, modules, and examples described herein.

The techniques described herein may be supported by various configurations of the computing device 2602 and are not limited to the specific examples of the techniques described herein. This functionality may also be implemented all or in part through use of a distributed system, such as over a “cloud” 2614 via a platform 2616 as described below.

The cloud 2614 includes and/or is representative of a platform 2616 for resources 2618. The platform 2616 abstracts underlying functionality of hardware (e.g., servers) and software resources of the cloud 2614. The resources 2618 may include applications and/or data that can be utilized while computer processing is executed on servers that are remote from the computing device 2602. Resources 2618 can also include services provided over the Internet and/or through a subscriber network, such as a cellular or Wi-Fi network.

The platform 2616 may abstract resources and functions to connect the computing device 2602 with other computing devices. The platform 2616 may also serve to abstract scaling of resources to provide a corresponding level of scale to encountered demand for the resources 2618 that are implemented via the platform 2616. Accordingly, in an interconnected device embodiment, implementation of functionality described herein may be distributed throughout the system 2600. For example, the functionality may be implemented in part on the computing device 2602 as well as via the platform 2616 that abstracts the functionality of the cloud 2614.

In some aspects, the techniques described herein relate to a method implemented in a diabetes management monitoring system, the method including: obtaining, from a sensor of the diabetes management monitoring system, diabetes management measurements measured for a user; identifying, based on the diabetes management measurements, multiple diabetes management feedbacks that correspond to the diabetes management measurements; determining one or more of the multiple diabetes management feedbacks having a highest ranking; and causing the determined diabetes management feedback having the highest ranking to be displayed.

In some aspects, the techniques described herein relate to a method, wherein the multiple diabetes management feedbacks correspond to different time periods of a day, and the determining includes ranking, for one time period of the day, ones of the multiple diabetes management feedbacks that correspond to the one time period of the day.

In some aspects, the techniques described herein relate to a method, wherein the multiple diabetes management feedbacks each correspond to one or more features or metrics for a time period, and the determining includes ranking each of the multiple diabetes management feedbacks based on the feature or metric corresponding to the diabetes management feedback.

In some aspects, the techniques described herein relate to a method, wherein the determining includes ranking each of the multiple diabetes management feedbacks based on a magnitude of an improvement in the diabetes management measurements corresponding to the feedback.

In some aspects, the techniques described herein relate to a method, wherein the determining includes determining that a diabetes management feedback that was caused to be displayed within a threshold number of preceding days is not the highest ranking diabetes management feedback.

In some aspects, the techniques described herein relate to a method, wherein the determining one or more of the multiple diabetes management feedbacks having the highest ranking includes: determining that a glucose level for a user was less than a threshold amount for at least a threshold amount of time during a time period of a day; and determining that feedback indicating the user was in a hypoglycemic range during the time period is the highest ranking diabetes management feedback.

In some aspects, the techniques described herein relate to a method, wherein the threshold amount includes 70 mg/dL, and the threshold amount of time includes 1 percent of the time period of the day.

In some aspects, the techniques described herein relate to a method, wherein the multiple diabetes management feedbacks include feedback identifying improvements in glucose measurements for a given time period over one or more previous days.

In some aspects, the techniques described herein relate to a method, wherein the multiple diabetes management feedbacks include feedback identifying a time period of a day during which glucose measurements were within an optimal range.

In some aspects, the techniques described herein relate to a method, wherein the multiple diabetes management feedbacks include feedback identifying sustained positive patterns of glucose measurements by the user.

In some aspects, the techniques described herein relate to a method, wherein the multiple diabetes management feedbacks include feedback identifying deviations in glucose measurements between time periods.

In some aspects, the techniques described herein relate to a method, wherein the multiple diabetes management feedbacks include feedback identifying potential behavior modification that a user could take to engage in beneficial diabetes management behavior.

In some aspects, the techniques described herein relate to a method, wherein the multiple diabetes management feedbacks include feedback identifying what a glucose level of the user would have been had a bout of physical activity not been engaged in by the user.

In some aspects, the techniques described herein relate to a device including: a display device; a feedback generation system, implemented at least in part in hardware, to obtain, from a sensor, diabetes management measurements measured for a user and to identify, based on the diabetes management measurements, multiple diabetes management feedbacks that correspond to the diabetes management measurements; and a feedback presentation system, implemented at least in part in hardware, to determine one of the multiple diabetes management feedbacks having a highest ranking and cause the determined diabetes management feedback having the highest ranking to be displayed.

In some aspects, the techniques described herein relate to a device, wherein the multiple diabetes management feedbacks include feedback identifying improvements in glucose measurements for a given time period over one or more previous days, feedback identifying a time period of the day during which glucose measurements were within an optimal range, and feedback identifying a sustained positive pattern of glucose measurements by the user.

In some aspects, the techniques described herein relate to a device, wherein the multiple diabetes management feedbacks include feedback identifying potential behavior modification that a user could take to engage in beneficial diabetes management behavior.

In some aspects, the techniques described herein relate to a device, wherein the multiple diabetes management feedbacks include feedback identifying what a glucose level of the user would have been had a bout of physical activity not been engaged in by the user.

In some aspects, the techniques described herein relate to a device, wherein the multiple diabetes management feedbacks include: feedback identifying improvements in glucose measurements for a given time period over one or more previous days, or feedback identifying a time period of the day during which glucose measurements were within an optimal range, or feedback identifying a sustained positive pattern of glucose measurements by the user; and feedback identifying potential behavior modification that a user could take to engage in beneficial diabetes management behavior.

In some aspects, the techniques described herein relate to a method implemented in a continuous glucose level monitoring system, the method including: obtaining, from a glucose sensor of the continuous glucose level monitoring system, first glucose measurements measured for a user for a first time period of multiple time periods of a current day, the glucose sensor being inserted at an insertion site of the user; identifying, based on the glucose measurements, multiple glucose management feedbacks that correspond to the glucose measurements; determining one of the multiple glucose feedbacks having a highest ranking; and causing the determined glucose feedback having the highest ranking to be displayed.

In some aspects, the techniques described herein relate to a method, wherein the multiple glucose management feedbacks include: feedback identifying improvements in glucose measurements for a given time period over one or more previous days, or feedback identifying a time period of the day during which glucose measurements were within an optimal range, or feedback identifying a sustained positive pattern of glucose measurements by the user; and feedback identifying potential behavior modification that a user could take to engage in beneficial diabetes management behavior.

### Conclusion

Although the systems and techniques have been described in language specific to structural features and/or methodological acts, it is to be understood that the systems and techniques defined in the appended claims are not necessarily limited to the specific features or acts described. Rather, the specific features and acts are disclosed as example forms of implementing the claimed subject matter.

What is claimed is:

1. A method implemented in a diabetes management monitoring system, the method comprising:
obtaining, from a sensor of the diabetes management monitoring system, diabetes management measurements measured for a user;
identifying, based on the diabetes management measurements, multiple diabetes management feedbacks that correspond to the diabetes management measurements;
determining one or more of the multiple diabetes management feedbacks having a highest ranking; and
causing the determined diabetes management feedback having the highest ranking to be displayed.

2. The method of claim 1, wherein the multiple diabetes management feedbacks correspond to different time periods of a day, and the determining includes ranking, for one time period of the day, ones of the multiple diabetes management feedbacks that correspond to the one time period of the day.

3. The method of claim 1, wherein the multiple diabetes management feedbacks each correspond to one or more features or metrics for a time period, and the determining includes ranking each of the multiple diabetes management feedbacks based on the feature or metric corresponding to the diabetes management feedback.

4. The method of claim 1, wherein the determining includes ranking each of the multiple diabetes management feedbacks based on a magnitude of an improvement in the diabetes management measurements corresponding to the feedback.

5. The method of claim 1, wherein the determining includes determining that a diabetes management feedback that was caused to be displayed within a threshold number of preceding days is not the highest ranking diabetes management feedback.

6. The method of claim 1, wherein the determining one or more of the multiple diabetes management feedbacks having the highest ranking includes:
determining that a glucose level for a user was less than a threshold amount for at least a threshold amount of time during a time period of a day; and
determining that feedback indicating the user was in a hypoglycemic range during the time period is the highest ranking diabetes management feedback.

7. The method of claim 6, wherein the threshold amount comprises 70 mg/dL, and the threshold amount of time comprises 1 percent of the time period of the day.

8. The method of claim 1, wherein the multiple diabetes management feedbacks include feedback identifying improvements in glucose measurements for a given time period over one or more previous days.

9. The method of claim 1, wherein the multiple diabetes management feedbacks include feedback identifying a time period of a day during which glucose measurements were within an optimal range.

10. The method of claim 1, wherein the multiple diabetes management feedbacks include feedback identifying sustained positive patterns of glucose measurements by the user.

11. The method of claim 1, wherein the multiple diabetes management feedbacks include feedback identifying deviations in glucose measurements between time periods.

12. The method of claim 1, wherein the multiple diabetes management feedbacks include feedback identifying potential behavior modification that a user could take to engage in beneficial diabetes management behavior.

13. The method of claim 1, wherein the multiple diabetes management feedbacks include feedback identifying what a glucose level of the user would have been had a bout of physical activity not been engaged in by the user.

14. A device comprising:
a display device;
a feedback generation system, implemented at least in part in hardware, to obtain, from a sensor, diabetes management measurements measured for a user and to identify, based on the diabetes management measurements, multiple diabetes management feedbacks that correspond to the diabetes management measurements; and
a feedback presentation system, implemented at least in part in hardware, to determine one of the multiple diabetes management feedbacks having a highest ranking and cause the determined diabetes management feedback having the highest ranking to be displayed.

15. The device of claim 14, wherein the multiple diabetes management feedbacks include feedback identifying improvements in glucose measurements for a given time period over one or more previous days, feedback identifying a time period of the day during which glucose measurements were within an optimal range, and feedback identifying a sustained positive pattern of glucose measurements by the user.

16. The device of claim 14, wherein the multiple diabetes management feedbacks include feedback identifying potential behavior modification that a user could take to engage in beneficial diabetes management behavior.

17. The device of claim 14, wherein the multiple diabetes management feedbacks include feedback identifying what a glucose level of the user would have been had a bout of physical activity not been engaged in by the user.

18. The device of claim 14, wherein the multiple diabetes management feedbacks include:
feedback identifying improvements in glucose measurements for a given time period over one or more previous days, or feedback identifying a time period of the day during which glucose measurements were within an optimal range, or feedback identifying a sustained positive pattern of glucose measurements by the user; and
feedback identifying potential behavior modification that a user could take to engage in beneficial diabetes management behavior.

19. A method implemented in a continuous glucose level monitoring system, the method comprising:
obtaining, from a glucose sensor of the continuous glucose level monitoring system, first glucose measurements measured for a user for a first time period of multiple time periods of a current day, the glucose sensor being inserted at an insertion site of the user;
identifying, based on the glucose measurements, multiple glucose management feedbacks that correspond to the glucose measurements;
determining one of the multiple glucose feedbacks having a highest ranking; and
causing the determined glucose feedback having the highest ranking to be displayed.

20. The method of claim 19, wherein the multiple glucose management feedbacks include:
feedback identifying improvements in glucose measurements for a given time period over one or more previous days, or feedback identifying a time period of the day during which glucose measurements were within an optimal range, or feedback identifying a sustained positive pattern of glucose measurements by the user; and
feedback identifying potential behavior modification that a user could take to engage in beneficial diabetes management behavior.
